(19) United States
(12) Patent Application Publication
(10) Pub. No.: US 2026/0035340 A1
(43) Pub. Date: Feb. 5, 2026

(54) INHIBITORS OF AKR1C3 AND METHODS OF USE

(71) Applicants: BOARD OF REGENTS OF THE UNIVERSITY OF NEBRASKA, Lincoln, NE (US); The Trustees of the University of Pennsylvania, Philadelphia, PA (US)

(72) Inventors: Paul Trippier, Papillion, NE (US); Krishnaiah Maddeboina, Charlotte, NC (US); Sravan Jonnalagadda, Omaha, NE (US); Trevor Penning, Springfield, PA (US)

(21) Appl. No.: 19/099,792

(22) PCT Filed: Aug. 4, 2023
(86) PCT No.: PCT/US2023/029511

§ 371 Date: Jan. 30, 2025

Related U.S. Application Data
(60) Provisional application No. 63/395,460, filed on Aug. 5, 2022.

(51) Int. Cl. C07C 233/65; A61K 31/192; A61K 31/357; A61K 31/36; A61K 31/403; A61K 31/437; A61K 31/47; A61K 31/498; A61K 31/5375; A61P 35/00; C07C 235/42; C07C 235/46; C07D 209/86; C07D 215/14; C07D 241/42; C07D 295/155; C07D 317/60; C07D 319/18; C07D 471/04

(52) U.S. Cl. CPC C07C 233/65; A61K 31/192; A61K 31/357; A61K 31/36; A61K 31/403; A61K 31/437; A61K 31/47; A61K 31/498; A61K 31/5375; A61P 35/00; C07C 235/42; C07C 235/46; C07D 209/86; C07D 215/14; C07D 241/42; C07D 295/155; C07D 317/60; C07D 319/18; C07D 471/04; C07C 2601/02

(57) ABSTRACT

Provided herein are compounds having a structure of formula (A), or pharmaceutically acceptable salts thereof, wherein R¹, R², R³, R⁴, R^{d} and n are described herein. Also provided herein are compositions comprising compounds of Formula (A) and methods of using compound of Formula (A) in treating, inhibiting, and/or preventing disease such as cancers (e.g., prostate cancer).

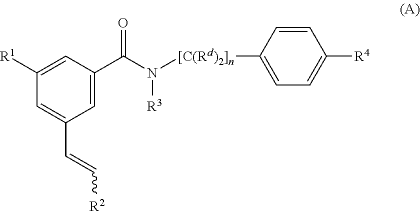

### STATEMENT OF U.S. GOVERNMENT SUPPORT

This invention was made with government support under R01 CA226436 awarded by the National Institutes of Health. The government has certain rights in the invention.

### BACKGROUND

Aldo-keto reductase (AKR) 1C3, also known as type 5 17β-hydroxysteroid dehydrogenase (17β-HSD) and prostaglandin [PG] F2α synthase is a member of the AKR1C subfamily, a member of a superfamily of NAD(P)H-linked oxidoreductases that reduce ketones on steroid and prostaglandin substrates to their corresponding secondary alcohols. Three highly homologous isoforms also participate in steroid hormone synthesis and metabolism; AKR1C1, AKR1C2 and AKR1C4, with varied stereo—and positional specificities for steroid substrates. Inhibition of androgen signalling and cell proliferation is mediated by the enzymes AKR1C1 and AKR1C2 (possessing >84% sequence homology to AKR1C3) which function to reduce the potent androgen 5α-dihydrotestosterone to the corresponding products 5α-androstane-3β, 17β-diol and 5α-androstane-3α, 17β-diol.

In the prostate, AKR1C3 converts 4-androstene-3,17-dione and 5α-androstane-3,17-dione to testosterone (T) and dihydrotestosterone (DHT) respectively, which are potent ligands for the androgen receptor (AR), a driving force for prostate cancer (PCa) development and progression. The enzyme is overexpressed at both the mRNA and protein levels in prostate tumors from castration-resistant prostate cancer (CRPC) patients but has low or undetectable expression in healthy prostate tissue. Reduction of AKR1C3 expression, or pharmacological inhibition significantly decreases the levels of T and DHT and androgen-dependent gene expression (prostate specific antigen (PSA)). AKR1C3 has been shown to contribute to chemotherapeutic resistance in CRPC since its expression is increased in prostate cancer cell lines resistant to conventional therapies (e.g., enzalutamide and abiraterone acetate). An emerging role of AKR1C3 to promote the stabilization of AR splice variant 7 (ARv7), a major determinant of enzalutamide resistance, further delineates its role as a central mediator of drug resistance in advanced prostate cancer. Given the structural similarities between AKR1C isoforms that perform different physiological roles, there is interest in developing selective inhibitors for AKR1C3.

### SUMMARY

There is a need for compounds that are selective inhibitors of AKR1C3 to treat conditions associated with aberrant AKR1C3 activity (e.g., cancer).

The disclosure provides compounds, or pharmaceutically acceptable salts thereof, having a structure of formula (A):

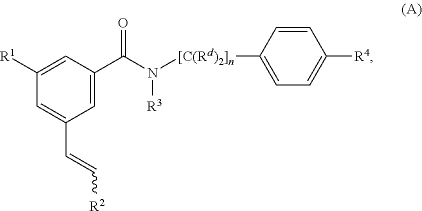

wherein R¹ is selected from substituted or unsubstituted C₆-C₁₂aryl, substituted or unsubstituted C₂-C₆alkynyl, substituted or unsubstituted C₁-C₆alkyl, C₂-C₆alkenyl, substituted or unsubstituted C₂-C₆alkenyloxy, halo, substituted or unsubstituted C₆-C₁₀aryl-C₁-C₃alkyleneoxy, C₆-C₁₀aryloxy, and substituted or unsubstituted C₅-C₁₀heteroaryl having 1-4 ring heteroatoms selected from N, O, and S; wherein R¹ is optionally substituted with one or more substituents selected from C₆-C₁₀aryl; 5-15 membered heteroaryl having 1-4 ring heteroatoms selected from N, O, and S; formyl, C₃-C₆cycloalkyl, halo, cycloheteroalkyl-alkylene, hydroxy-C₁-C₃alkylene, methoxymethyl, phenyoxy, cyano, C₁-C₆alkyl, C₁-C₆haloalkyl, and 5-7 membered fused cycloheteroalkyl having 1 to 3 ring heteroatoms selected from N, O, and S; R² is selected from CO₂R^{a}, CONR^{b}R^{c}, wherein each R^{a}, R^{b}, and R^{c} is independently H or C₁-C₆alkyl; each R³ and R⁴ is independently H or C₁-C₆alkyl; each R^{d} is independently H or C₁-C₃alkyl; and n is 1, 2, or 3.

The disclosure also provides pharmaceutical compositions comprising the disclosed compounds or pharmaceutically acceptable salts thereof and a pharmaceutically acceptable carrier or excipient. The disclosure further provides methods of treating, inhibiting, and/or diseases in a subject in need thereof, wherein the method comprises administering to the subject a therapeutically effective amount of the disclosed compounds or pharmaceutically acceptable salts thereof.

### BRIEF DESCRIPTION OF THE DRAWINGS

FIG. 1 shows conventional AKR1C3 inhibitors.

FIG. 2a shows a two-dimensional representation of the binding site and amino acid residues participating in interactions with compound A1-r.

FIG. 2b shows binding interactions of compound A1-r with AKR1C3 (PDB ID: 3UG8).

FIG. 2c shows a two-dimensional representation of the binding site and amino acid residues participating in interactions with compound A1-a.

FIG. 2d shows binding interactions of compound A1-a with AKR1C3 (PDB ID: 3UG8).

FIG. 3A shows the in vitro stability of compound A1-r in mouse plasma, simulated gastric fluid (SGF) and simulated intestinal fluid (SIF).

FIG. 3B shows the in vitro stability of compound 4r in mouse plasma, simulated gastric fluid (SGF) and simulated intestinal fluid (SIF).

FIG. 4A shows the in vitro stability of compound A1-r in mouse liver microsomes (MLM), human liver microsomes (HLM), and negative control.

FIG. 4B shows the in vitro stability of compound 4r in mouse liver microsomes (MLM), human liver microsomes (HLM), and negative control.

FIG. 4C shows the in vitro formation of compound A1-r from compound A1′-r in mouse liver microsomes (MLM), human liver microsomes (HLM), and negative control.

FIG. 5A shows in vivo plasma concentration time profile of compounds 4r and A1-r_{metab} after 10 mg/kg post oral administration of 4r in mice.

FIG. 5B shows in vivo plasma concentration time profile of compound A1-r following oral administration of compound A1-r (10 mg/kg) in mice.

FIG. 6A shows the Impact of vehicle control and compound 4r treatment at 25 and 50 mg/Kg IP dosing once per day on 22Rv1 tumor growth in NSG mice, values represent the mean of n=6 animals±SEM. **, p<0.01; ****, p<0.0001; by two-way ANOVA.

FIG. 6B shows the body weight of NSG 22Rv1 tumor-bearing mice (n=6) after treatment with compound 4r at the indicated dose, values represent the mean of n=6 animals±SEM.

FIG. 6C shows the excised tumors from vehicle treated NSG 22Rv1 tumor-bearing mice.

FIG. 6D shows the excised tumors from compound 4r (50 mg/Kg) treated NSG 22Rv1 tumor-bearing mice. Statistical analysis (Grubbs test, Z=1.93, p<0.05) indicated one of the tumor-bearing vehicle mice was an outlier and those data have been omitted in the plot but excised tumor is still shown in FIG. 6C.

### DETAILED DESCRIPTION

The present disclosure provides compounds useful for treating, inhibiting, and/or preventing diseases associated with aberrant AKR1C3 activity.

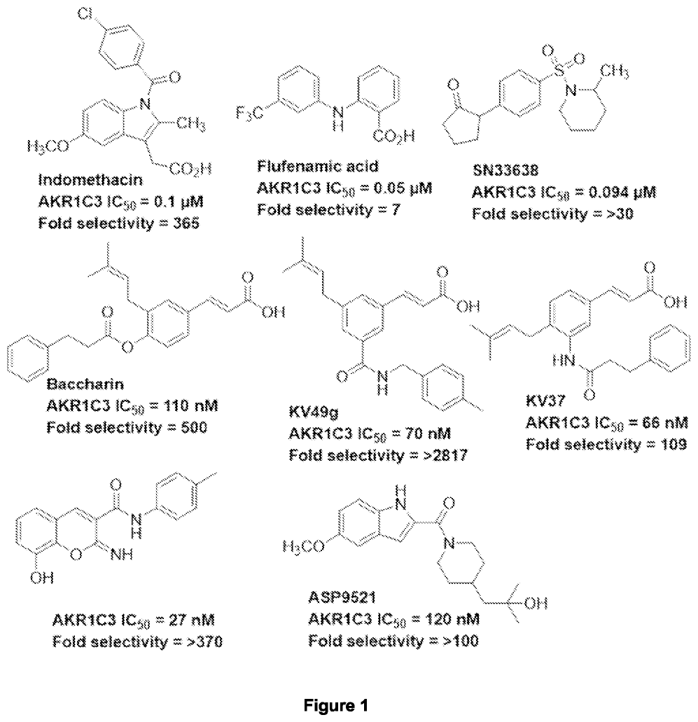

The compounds and pharmaceutical salts disclosed herein provide several advantages over conventional compounds and treatment regimens. Various synthetic and natural inhibitors of AKR1C3 are known. Although nonsteroidal anti-inflammatory drug (NSAID) analogues have been reported to have some degree of activity and selectivity for AKR1C3 over other AKR1C isoforms, these compounds suffer from poor pharmacokinetic properties and/or insufficient selectivity resulting in none of these compounds progressing to the clinic. Other challenges include the development of resistance or unwanted side-effects. The reported compounds include including analogues of naproxen, indomethacin, and flufenamic acid (e.g., SN33638, baccharin, KV49g, KV37, ASP9521) as shown in FIG. 1.

### Compounds of the Disclosure

The disclosure provides compounds having a structure of formula (A), or a pharmaceutically acceptable salts thereof:

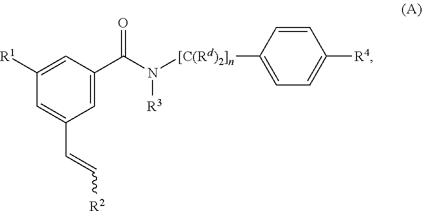

wherein R¹, R², R³, R⁴ R^{d}, and n are described herein.

### R¹

The compounds disclosed herein comprise a R¹ moiety, wherein R¹ is selected from substituted or unsubstituted C₆-C₁₂aryl, substituted or unsubstituted C₂-C₆alkynyl, substituted or unsubstituted C₁-C₆alkyl, C₂-C₆alkenyl, substituted or unsubstituted C₂-C₆alkenyloxy, halo, substituted or unsubstituted C₆-C₁₀aryl-C₁-C₃alkyleneoxy, C₆-C₁₀aryloxy, and substituted or unsubstituted C₅-C₁₀heteroaryl having 1-4 ring heteroatoms selected from N, O, and S.

For example, R¹ can be selected from substituted or unsubstituted C₆-C₁₂aryl, substituted or unsubstituted C₂-C₆alkynyl, substituted or unsubstituted C₁-C₆alkyl, C₂-C₆alkenyl, substituted or unsubstituted C₂-C₆alkenyloxy, halo, substituted or unsubstituted C₆-C₁₀aryl-C₁-C₃alkyleneoxy, and substituted or unsubstituted C₅-C₁₀heteroaryl having 1-4 ring heteroatoms selected from N, O, and S.

For example, R¹ is selected from substituted or unsubstituted C₆-C₁₂aryl, C₂-C₆alkenyl, substituted or unsubstituted C₂-C₆alkenyloxy, C₆-C₁₀aryloxy, and C₅-C₁₀heteroaryl having 1-4 ring heteroatoms selected from N, O, and S. For example, R¹ can be selected from substituted or unsubstituted C₆-C₁₂aryl and substituted or unsubstituted C₅-C₁₀heteroaryl having 1-4 ring heteroatoms selected from N, O, and S.

For example, R¹ can be selected from

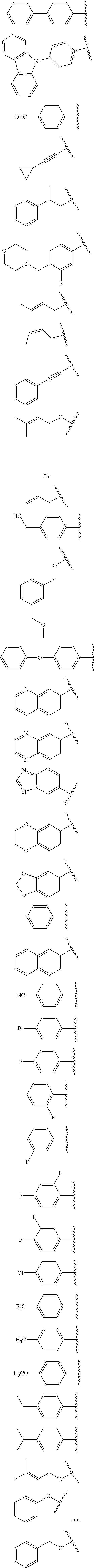

For example, R¹ can be selected from selected from:

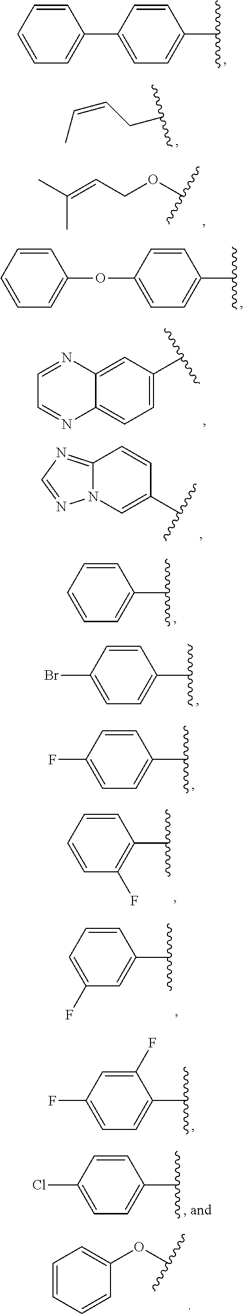

For example, R¹ can be selected from:

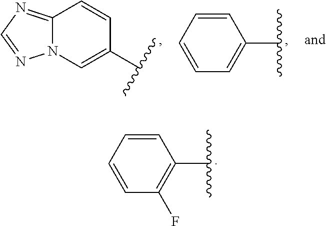 [SMILES: *c1ccc2ncnn2c1 ; *c1ccccc1 ; *c1ccccc1F]

For example, in some instances R¹ is

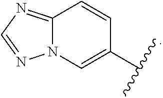 [SMILES: *c1ccc2ncnn2c1]

For example, in some instances R¹ is

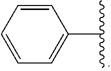 [SMILES: *c1ccccc1]

R¹ can be further substituted. For example, R¹ is optionally substituted with one or more substituents selected from C₆-C₁₀aryl; 5-15 membered heteroaryl having 1-4 ring heteroatoms selected from N, O, and S; formyl, C₃-C₆cycloalkyl, halo, cycloheteroalkyl-alkylene, hydroxy-C₁-C₃alkylene, methoxymethyl, phenyoxy, cyano, C₁-C₆alkyl, C₁-C₆haloalkyl, and 5-7 membered fused cycloheteroalkyl having 1 to 3 ring heteroatoms selected from N, O, and S. The R¹ moiety can be further substituted with one or more substituents selected from C₆-C₁₀aryl; 5-15 membered heteroaryl having 1-4 ring heteroatoms selected from N, O, and S; formyl, C₃-C₆cycloalkyl, halo, cycloheteroalkyl-alkylene, hydroxy-C₁-C₃alkylene, methoxymethyl, phenyoxy, cyano, C₁-C₆alkyl, C₁-C₆haloalkyl, and 5-7 membered fused cycloheteroalkyl having 1 to 3 ring heteroatoms selected from N, O, and S. In R¹ can be further substituted with one or more substituents selected from C₆-C₁₀aryl, C₆-C₁₀aryloxy, and halo. For example, R¹ can be further substituted with halo.

R² and R^{a}, R^{b}, and R^{c}

The compounds disclosed herein comprise a R² moiety, wherein R² is selected from CO₂R^{a}, CONR^{b}R^{c}, wherein each R^{a}, R^{b}, and R^{c} is independently H or C₁-C₆alkyl. For example, R² is selected from CO₂H, CO₂CH₃, CO₂CH₂CH₃, CO₂(CH₂)₂CH₃, CO₂C(CH₃)₃, CONH₂, CONHCH₃, CON(CH₃)₂, and CN. For example, R² is CO₂H or CO₂CH₃. For example, R² is CO₂H. For example, R² is CO₂CH₃.

The double bond to which R² is attached can have the E-configuration. In some embodiments, the double bond to which R² is attached can have the Z-configuration.

R³ and R⁴

The compounds disclosed herein comprise a R³ and R⁴ moiety, wherein R³ and R⁴ are independently H or C₁-C₆alkyl. For example, R³ can be H. For example, R⁴ can be H. For example, R⁴ can be CH₃.

### R^{d}

The compounds disclosed herein comprise a R^{d} moiety, wherein each R^{d} is independently H or C₁-C₃alkyl. For example, each R^{d} can be H. For example, at least one R^{d} can be C₁-C₃alkyl. For example, one or more R^{d} is CH₃ or CH₂CH₃. For example, one or more R^{d} can be CH₃. For example, one or more R^{d} can be CH₂CH₃.

n

The compounds disclosed herein comprise the moiety —[C(R^{d})2]ₙ-, wherein n is 1, 2, or 3.

### Compounds (A-1), (A-2), (A-3) and (A-4)

The disclosure also provides compounds of formula (A) having the structure of formula (A-1)

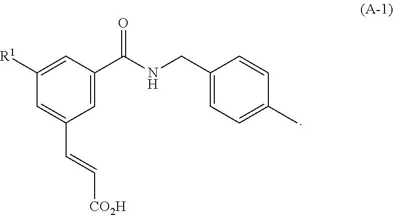

Compounds of formula (A) can have the structure of formula (A-2)

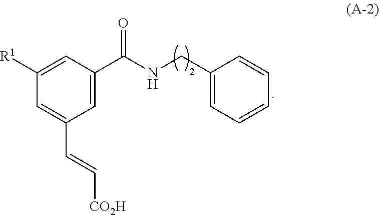

Compounds of formula (A) can have the structure of formula (A-3)

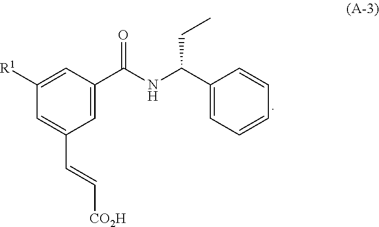

Compounds of formula (A) can have the structure of formula (A-4)

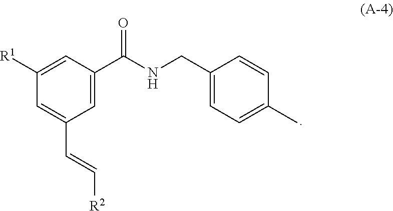

For example, the disclosure provides a compound of formula (A), or a pharmaceutically acceptable salt thereof, wherein the compound of formula (A) is selected from the structures listed in Table 1, Table 3, and/or Table 4, as shown in the Examples.

### Definitions

As used herein, the term “alkyl” refers to straight chained and branched saturated hydrocarbon groups containing one to thirty carbon atoms, for example, one to twenty carbon atoms, or one to ten carbon atoms. The term Cₙ means the alkyl group has “n” carbon atoms. For example, C4 alkyl refers to an alkyl group that has 4 carbon atoms. C₁-C₈ alkyl refers to an alkyl group having a number of carbon atoms encompassing the entire range (e.g., 1 to 8 carbon atoms), as well as all subgroups (e.g., 1-8, 2-8, 3-8, 4-8, 5-8, 6-8, 7-8, 1, 2, 3, 4, 5, 6, 7, and 8 carbon atoms). Nonlimiting examples of alkyl groups include, methyl, ethyl, n-propyl, isopropyl, n-butyl, sec-butyl (2-methylpropyl), t-butyl (1,1-dimethylethyl), 3,3-dimethylpentyl, and 2-ethylhexyl. Unless otherwise indicated, an alkyl group can be an unsubstituted alkyl group or a substituted alkyl group.

The term “alkylene” used herein refers to an alkyl group having a substituent. For example, the term “alkylenehalo” refers to an alkyl group substituted with a halo group. For example, an alkylene group can be —CH₂CH₂— or —CH₂—. The term Cₙ means the alkylene group has “n” carbon atoms. For example, C₁₋₁₈ alkylene refers to an alkylene group having a number of carbon atoms encompassing the entire range, as well as all subgroups, as previously described for “alkyl” groups. Unless otherwise indicated, an alkylene group can be an unsubstituted alkylene group or a substituted alkylene group.

The term “alkenyl” used herein refers to an unsaturated aliphatic group analogous in length and possible substitution to an alkyl group described above, but that contains at least one double bond. For example, the term “alkenyl” includes straight chain alkenyl groups (e.g., ethenyl, propenyl, butenyl, pentenyl, hexenyl, heptenyl, octenyl, nonenyl, decenyl), and branched alkenyl groups. For example, a straight chain or branched alkenyl group can have six or fewer carbon atoms in its backbone (e.g., C₂-C₆ for straight chain, C₃-C₆ for branched chain). The term “C₂-C₆” includes chains having a number of carbon atoms encompassing the entire range (e.g., 2 to 6 carbon atoms), as well as all subgroups (e.g., 2-6, 2-5, 2-4, 3-6, 2, 3, 4, 5, and 6 carbon atoms). The term “C₃-C₆” includes chains having a number of carbon atoms encompassing the entire range (e.g., 3 to 6 carbon atoms), as well as all subgroups (e.g., 3-6, 3-5, 3-4, 3, 4, 5, and 6 carbon atoms). Unless otherwise indicated, an alkenyl gr

The term “alkenylene” used herein refers to an alkenyl group having a substituent. For example, the term “alkenylenehalo” refers to an alkyl group substituted with a halo group. For example, an alkylene group can be —CH═CH—. The term Cₙ means the alkenylene group has “n” carbon atoms. For example, C₂-C₆ alkenylene refers to an alkenylene group having a number of carbon atoms encompassing the entire range, as well as all subgroups, as previously described for “alkenyl” groups. Unless otherwise indicated, an alkenylene group can be an unsubstituted alkenylene group or a substituted alkenylene group.

As used herein, an alkylene which is “interrupted” is understood to be an alkylene group in which at one or more (e.g., 1-5, 1-4, 1-3, 1-2, 1, 2, 3, 4, or 5) positions on the alkylene chain is inserted a group selected from one or more of (i) non-adjacent heteroatom(s) selected from O, S, and NRN, (ii) C(O)NRN, and (iii) NRNC(O). The interruptions can be consecutive for various combinations of these interrupting groups (e.g., a heteroatom next to a C(O)NRN moiety), except that two heteroatoms cannot be adjacent or consecutive to each other.

As used herein an alkylene which is interrupted with “one or more” groups is understood to be interrupted with from 1 to n−1 groups, wherein n is the number of carbon atoms in the alkylene chain. For example, a C₆alkylene which is optionally interrupted with one or more groups can be interrupted with one, two, three, four, or five groups.

The term “alkynyl” used herein refers to an unsaturated aliphatic group analogous in length and possible substitution to an alkyl group described above, but that contains at least one triple bond. For example, the term “alkynyl” includes straight chain alkynyl groups (e.g., ethynyl, propynyl, butynyl, pentynyl, hexynyl, heptynyl, octynyl), and branched alkynyl groups. For example, a straight chain or branched alkynyl group can have eight or fewer carbon atoms in its backbone (e.g., C₂-C₈ for straight chain, C₄-C₈ for branched chain). The term “C₂-C₈” includes chains having a number of carbon atoms encompassing the entire range (e.g., 2 to 8 carbon atoms), as well as all subgroups (e.g., 2-6, 2-5, 2-4, 3-6, 2, 3, 4, 5, and 6 carbon atoms). The term “C₄-C₈” includes chains having a number of carbon atoms encompassing the entire range (e.g., 4 to 8 carbon atoms), as well as all subgroups (e.g., 4-6, 4-5, 4, 5, and 6 carbon atoms). Unless otherwise indicated, an alkynyl group can be an unsubstituted alkynyl group or a substituted alkynyl group.

The term “alkynylene” used herein refers to an alkynyl group having a substituent. The term Cₙ means the alkynylene group has “n” carbon atoms. For example, C₂-C₈ alkynylene refers to an alkynylene group having a number of carbon atoms encompassing the entire range, as well as all subgroups, as previously described for “alkynyl” groups. Unless otherwise indicated, an alkynylene group can be an unsubstituted alkynylene group or a substituted alkynylene group.

As used herein, the term “cycloalkyl” refers to an aliphatic cyclic hydrocarbon group containing three to eleven carbon atoms (e.g., 3, 4, 5, 6, 7, 8, 9, 10, or 11 carbon atoms). The term Cₙ means the cycloalkyl group has “n” carbon atoms. For example, C5 cycloalkyl refers to a cycloalkyl group that has 5 carbon atoms in the ring. C₆-C11 cycloalkyl refers to cycloalkyl groups having a number of carbon atoms encompassing the entire range (e.g., 6 to 11 carbon atoms), as well as all subgroups (e.g., 6-7, 6-8, 7-8, 6-9, 6, 7, 8, 9, 10, and 11 carbon atoms). Nonlimiting examples of cycloalkyl groups include cyclopropyl, cyclobutyl, cyclopentyl, cyclohexyl, cycloheptyl, and cyclooctyl. Unless otherwise indicated, a cycloalkyl group can be an unsubstituted cycloalkyl group or a substituted cycloalkyl group. The cycloalkyl groups described herein can be isolated or fused to another cycloalkyl group. When a cycloalkyl group is fused to another cycloalkyl group, then each of the cycloalkyl groups can contain three to twelve carbon atoms unless specified otherwise. Unless otherwise indicated, a cycloalkyl group can be unsubstituted or substituted.

The term “cycloalkenyl” is defined similarly as “cycloalkyl” except that the ring comprises at least one double bond, without being aromatic. The cycloalkenyl groups described herein can be isolated or fused to another cycloalkenyl group. Unless otherwise indicated, a cycloalkenyl group can be unsubstituted or substituted.

As used herein, the term “heterocycloalkyl” is defined similarly as cycloalkyl, except the ring contains one to three heteroatoms independently selected from oxygen, nitrogen, and sulfur. In particular, the term “heterocycloalkyl” refers to a ring containing a total of three to eleven atoms (e.g., three to seven, or five to eleven), of which 1, 2, 3 or three of those atoms are heteroatoms independently selected from the group consisting of oxygen, nitrogen, and sulfur, and the remaining atoms in the ring are carbon atoms. Nonlimiting examples of heterocycloalkyl groups include piperdine, pyrazolidine, tetrahydrofuran, tetrahydropyran, dihydrofuran, morpholine, and the like. The heterocycloalkyl groups described herein can be isolated or fused to another heterocycloalkyl group. Heterocycloalkyl groups can be saturated or partially unsaturated ring systems. Unless otherwise indicated, a heterocycloalkyl group can be unsubstituted or substituted.

As used herein, the term “aryl” refers to a monocyclic aromatic hydrocarbon group, such as phenyl or a bicyclic aromatic hydrocarbon group, such as naphthyl. Unless otherwise indicated, an aryl group can be unsubstituted or substituted with one or more groups. Aryl groups can be isolated (e.g., phenyl) or fused to another aryl group (e.g., naphthyl, anthracenyl), a cycloalkyl group (e.g. tetraydronaphthyl), a heterocycloalkyl group, and/or a heteroaryl group. Exemplary aryl groups include, but are not limited to, phenyl, chlorophenyl, methylphenyl, methoxyphenyl, trifluoromethylphenyl, nitrophenyl, 2,4-methoxychlorophenyl, and the like. Throughout, the abbreviation “Ph” refers to phenyl and “Bn” refers to benzyl (i.e., CH₂phenyl).

As used herein, the term “heteroaryl” refers to a monocyclic or bicyclic aromatic ring having 5 to 14 total ring atoms, and containing one to three heteroatoms selected from nitrogen, oxygen, and sulfur atom in the aromatic ring. Unless otherwise indicated, a heteroaryl group can be unsubstituted or substituted. Examples of heteroaryl groups include, but are not limited to, thienyl, furyl, pyridyl, pyrrolyl, oxazolyl, triazinyl, triazolyl, isothiazolyl, isoxazolyl, imidazolyl, pyrazinyl, pyrimidinyl, thiazolyl, and thiadiazolyl.

As used herein, the term “oxo” refers to a ═O group.

As used herein, the term “cyano” refers to a —CN group. The terms “cyano” and “nitrile” may be used interchangeably.

As used herein, the term “halo” refers to a F (fluoro), Cl (chloro), Br (bromo), or I (iodo) group.

As used herein, the term “substituted,” when used to modify a chemical functional group, refers to the replacement of at least one hydrogen radical on the functional group with a substituent. Substituents can include, but are not limited to, alkyl, cycloalkyl, alkenyl, cycloalkenyl, alkynyl, heterocycloalkyl, aryl, heteroaryl, hydroxyl, oxy, alkoxy, heteroalkoxy, ester, thioester, carboxy, cyano, nitro, amino, amido, acetamide, and halo (e.g., fluoro, chloro, bromo, or iodo). When a chemical functional group includes more than one substituent, the substituents can be bound to the same carbon atom or to two or more different carbon atoms.

As used herein, the phrase “optionally substituted” means unsubstituted (e.g., substituted with a H) or substituted. As used herein, the term “substituted” means that a hydrogen atom is removed and replaced by a substituent. It is understood that substitution at a given atom is limited by valency. The use of a substituent (radical) prefix name such as alkyl without the modifier “optionally substituted” or “substituted” is understood to mean that the particular substituent is unsubstituted.

### Pharmaceutically Acceptable Salts

The compounds disclosed herein can be in the form of a pharmaceutically acceptable salt. As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al. describe pharmaceutically acceptable salts in detail in J. Pharmaceutical Sciences, 1977, 66, 1-19, which is incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, trifluoroacetic acid, oxalic acid, maleic acid, tartaric acid, citric acid, succinic acid or malonic acid or by using other methods used in the art such as ion exchange. Other illustrative pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, glutamate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts of compounds containing a carboxylic acid or other acidic functional group can be prepared by reacting with a suitable base. Such salts include, but are not limited to, alkali metal, alkaline earth metal, aluminum salts, ammonium, N⁺(C₁₋₄alkyl)₄ salts, and salts of organic bases such as trimethylamine, triethylamine, morpholine, pyridine, piperidine, picoline, dicyclohexylamine, N,N′-dibenzylethylenediamine, 2-hydroxyethylamine, bis-(2-hydroxyethyl)amine, tri-(2-hydroxyethyl)amine, procaine, dibenzylpiperidine, dehydroabietylamine, N,N′-bisdehydroabietylamine, glucamine, N-methylglucamine, collidine, quinine, quinoline, and basic amino acids such as lysine and arginine. This invention also envisions the quaternization of any basic nitrogen-containing groups of the compounds disclosed herein. Water or oil-soluble or dispersible products may be obtained by such quaternization. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate.

### Pharmaceutical Compositions

The compounds described herein can be administered to a subject in a therapeutically effective amount (e.g., in an amount sufficient to prevent or relieve the symptoms of a parasitic disease). The compounds can be administered alone or as part of a pharmaceutically acceptable composition or formulation. In addition, the compounds can be administered all at once, multiple times, or delivered substantially uniformly over a period of time. It is also noted that the dose of the compound can be varied over time.

The methods can comprise administering, e.g., from about 0.1 mg/kg up to about 100 mg/kg of compound or more, depending on the factors mentioned above. In other embodiments, the dosage ranges from 1 mg/kg up to about 100 mg/kg; or 5 mg/kg up to about 100 mg/kg; or 10 mg/kg up to about 100 mg/kg. Some conditions require prolonged treatment, which may or may not entail administering lower doses of compound over multiple administrations. If desired, a dose of the compound is administered as two, three, four, five, six or more sub-doses administered separately at appropriate intervals throughout the day, optionally, in unit dosage forms. The treatment period will depend on the particular condition being treated, and may last one day to several months.

In some embodiments, the methods comprise administering a compound of Formula (I) at a dosage of 100 mg/kg, or 50 mg/kg, or 25 mg/kg, or 12.5 mg/kg, or 6.25 mg/kg, or 1 mg/kg.

A particular administration regimen for a particular subject will depend, in part, upon the compound, the amount of compound administered, the route of administration, and the cause and extent of any side effects. The amount of compound administered to a subject (e.g., a mammal, such as a human) in accordance with the disclosure should be sufficient to effect the desired response over a reasonable time frame. Dosage typically depends upon the route, timing, and frequency of administration. Accordingly, the clinician titers the dosage and modifies the route of administration to obtain the optimal therapeutic effect, and conventional range-finding techniques are known to those of ordinary skill in the art.

Pharmaceutical compositions in accordance with the disclosure can comprise a compound of formula (A) as disclosed herein or a pharmaceutically acceptable salt thereof, and a pharmaceutically acceptable carrier or excipient. For example, the disclosed pharmaceutical compositions comprise a compound of formula (A1), formula (A2), formula (A3), or formula (A4), or pharmaceutically acceptable salts thereof.

In some embodiments, the pharmaceutical compositions comprise a compound of Formula (A1) or a pharmaceutically acceptable salt thereof. In some embodiments, the pharmaceutical compositions comprise a compound of Formula (A1), or a pharmaceutically acceptable salt thereof.

In some embodiments, the pharmaceutical compositions comprise a compound listed in Table 1, or a pharmaceutically acceptable salt thereof. In some embodiments, the pharmaceutical compositions comprise a compound listed in Table 3, or a pharmaceutically acceptable salt thereof. In some embodiments, the pharmaceutical compositions comprise a compound listed in Table 4, or a pharmaceutically acceptable salt thereof.

As used herein, the terms carrier or excipient are used interchangeably unless otherwise specified. Accordingly, a pharmaceutically acceptable carrier or excipient refers to any pharmaceutically acceptable additive, carrier, diluent, adjuvant, or other ingredient, other than the active pharmaceutical ingredient (API).

Suitable methods of administering a physiologically-acceptable composition, such as a pharmaceutical composition comprising the compounds disclosed herein (e.g., compounds of formula (A-1), formula (A-2), formula (A-3), or formula (A-4), or pharmaceutically acceptable salts thereof), are well known in the art. Although more than one route can be used to administer a compound, a particular route can provide a more immediate and more effective reaction than another route. Depending on the circumstances, a pharmaceutical composition comprising the compound is applied or instilled into body cavities, absorbed through the skin or mucous membranes, ingested, inhaled, and/or introduced into circulation. For example, in certain circumstances, it will be desirable to deliver a pharmaceutical composition comprising the agent orally, through injection by intravenous, intraperitoneal, intracerebral (intra-parenchymal), intracerebroventricular, intramuscular, intra-ocular, intraarterial, intraportal, intralesional, intramedullary, intrathecal, intraventricular, transdermal, subcutaneous, intraperitoneal, intranasal, enteral, topical, sublingual, urethral, vaginal, or rectal means, by sustained release systems, or by implantation devices. If desired, the compound is administered regionally via intrathecal administration, intracerebral (intra-parenchymal) administration, intracerebroventricular administration, or intraarterial or intravenous administration feeding the region of interest. Alternatively, the composition is administered locally via implantation of a membrane, sponge, or another appropriate material onto which the desired compound has been absorbed or encapsulated. Where an implantation device is used, the device is, in one aspect, implanted into any suitable tissue or organ, and delivery of the desired compound is, for example, via diffusion, timed-release bolus, or continuous administration.

To facilitate administration, the compound is, in various aspects, formulated into a physiologically-acceptable composition comprising a carrier (e.g., vehicle, adjuvant, or diluent). The particular carrier employed is limited only by chemico-physical considerations, such as solubility and lack of reactivity with the compound, and by the route of administration. Physiologically-acceptable carriers are well known in the art.

Illustrative pharmaceutical forms suitable for injectable use include sterile aqueous solutions or dispersions and sterile powders for the extemporaneous preparation of sterile injectable solutions or dispersions (for example, see U.S. Pat. No. 5,466,468). Injectable formulations are further described in, e.g., Pharmaceutics and Pharmacy Practice, J. B. Lippincott Co., Philadelphia. Pa., Banker and Chalmers, eds., pages 238-250 (1982), and ASHP Handbook on Injectable Drugs, Toissel, 4th ed., pages 622-630 (1986)). A pharmaceutical composition comprising the compound is, in one aspect, placed within containers, along with packaging material that provides instructions regarding the use of such pharmaceutical compositions. Generally, such instructions include a tangible expression describing the reagent concentration, as well as, in certain embodiments, relative amounts of excipient ingredients or diluents (e.g., water, saline or PBS) that may be necessary to reconstitute the pharmaceutical composition.

Compositions suitable for parenteral injection may comprise physiologically acceptable sterile aqueous or nonaqueous solutions, dispersions, suspensions, or emulsions, and sterile powders for reconstitution into sterile injectable solutions or dispersions. Examples of suitable aqueous and nonaqueous carriers, diluents, solvents, or vehicles include water, ethanol, polyols (propylene glycol, polyethylene glycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions, and by the use of surfactants.

These compositions may also contain adjuvants such as preserving, wetting, emulsifying, and dispersing agents. Microorganism contamination can be prevented by adding various antibacterial and antifungal agents, for example, parabens, chlorobutanol, phenol, sorbic acid, and the like. It may also be desirable to include isotonic agents, for example, sugars, sodium chloride, and the like. Prolonged absorption of injectable pharmaceutical compositions can be brought about by the use of agents delaying absorption, for example, aluminum monostearate and gelatin.

Solid dosage forms for oral administration include capsules, tablets, powders, and granules. In such solid dosage forms, the active compound is admixed with at least one inert customary excipient (or carrier) such as sodium citrate or dicalcium phosphate or (a) fillers or extenders, as for example, starches, lactose, sucrose, mannitol, and silicic acid; (b) binders, as for example, carboxymethylcellulose, alginates, gelatin, polyvinylpyrrolidone, sucrose, and acacia; (c) humectants, as for example, glycerol; (d) disintegrating agents, as for example, agar-agar, calcium carbonate, potato or tapioca starch, alginic acid, certain complex silicates, and sodium carbonate; (a) solution retarders, as for example, paraffin; (f) absorption accelerators, as for example, quaternary ammonium compounds; (g) wetting agents, as for example, cetyl alcohol and glycerol monostearate; (h) adsorbents, as for example, kaolin and bentonite; and (i) lubricants, as for example, talc, calcium stearate, magnesium stearate, solid polyethylene glycols, sodium lauryl sulfate, or mixtures thereof. In the case of capsules, and tablets, the dosage forms may also comprise buffering agents. Solid compositions of a similar type may also be used as fillers in soft and hard filled gelatin capsules using such excipients as lactose or milk sugar, as well as high molecular weight polyethylene glycols, and the like.

Solid dosage forms such as tablets, dragees, capsules, pills, and granules can be prepared with coatings and shells, such as enteric coatings and others well known in the art. The solid dosage forms may also contain opacifying agents. Further, the solid dosage forms may be embedding compositions, such that they release the active compound or compounds in a certain part of the intestinal tract in a delayed manner. Examples of embedding compositions that can be used are polymeric substances and waxes. The active compound can also be in micro-encapsulated form, optionally with one or more excipients.

Liquid dosage forms for oral administration include pharmaceutically acceptable emulsions, solutions, suspensions, syrups, and elixirs. In addition to the active compounds, the liquid dosage form may contain inert diluents commonly used in the art, such as water or other solvents, solubilizing agents and emulsifiers, as for example, ethyl alcohol, isopropyl alcohol, ethyl carbonate, ethyl acetate, benzyl alcohol, benzyl benzoate, propylene glycol, 1,3-butylene glycol, dimethylformamide, oils, in particular, cottonseed oil, groundnut oil, corn germ oil, olive oil, castor oil, and sesame seed oil, glycerol, tetrahydrofurfuryl alcohol, polyethylene glycols and fatty acid esters of sorbitan, or mixtures of these substances, and the like.

Besides such inert diluents, the composition can also include adjuvants, such as wetting agents, emulsifying and suspending agents, sweetening, flavoring, and perfuming agents. Suspensions, in addition to the active compound, may contain suspending agents, as for example, ethoxylated isostearyl alcohols, polyoxyethylene sorbitol and sorbitan esters, microcrystalline cellulose, aluminum metahydroxide, bentonite, agar-agar, and tragacanth, or mixtures of these substances, and the like.

Compositions for rectal administration are preferably suppositories, which can be prepared by mixing the compounds of the disclosure with suitable non-irritating excipients or carriers such as cocoa butter, polyethylene glycol or a suppository wax, which are solid at ordinary room temperature, but liquid at body temperature, and therefore, melt in the rectum or vaginal cavity and release the active component.

The pharmaceutical compositions used in the methods of the disclosure may be formulated in micelles or liposomes. Such formulations include sterically stabilized micelles or liposomes and sterically stabilized mixed micelles or liposomes. Such formulations can facilitate intracellular delivery, since lipid bilayers of liposomes and micelles are known to fuse with the plasma membrane of cells and deliver entrapped contents into the intracellular compartment.

Upon formulation, solutions will be administered in a manner compatible with the dosage formulation and in such amount as is therapeutically effective. The formulations are easily administered in a variety of dosage forms such as injectable solutions, drug release capsules and the like. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration.

The frequency of dosing will depend on the pharmacokinetic parameters of the agents and the routes of administration. The optimal pharmaceutical formulation will be determined by one of skill in the art depending on the route of administration and the desired dosage. See, for example, Remington's Pharmaceutical Sciences, 18th Ed. (1990) Mack Publishing Co., Easton, PA, pages 1435-1712, incorporated herein by reference. Such formulations may influence the physical state, stability, rate of in vivo release and rate of in vivo clearance of the administered agents. Depending on the route of administration, a suitable dose may be calculated according to body weight, body surface areas or organ size. Further refinement of the calculations necessary to determine the appropriate treatment dose is routinely made by those of ordinary skill in the art without undue experimentation, especially in light of the dosage information and assays disclosed herein, as well as the pharmacokinetic data observed in animals or human clinical trials.

The precise dosage to be employed depends upon several factors including the host, whether in veterinary medicine or human medicine, the nature and severity of the condition, e.g., disease or disorder, being treated, the mode of administration and the particular active substance employed. The compounds may be administered by any conventional route, in particular enterally, and, in one aspect, orally in the form of tablets or capsules. Administered compounds can be in the free form or pharmaceutically acceptable salt form as appropriate, for use as a pharmaceutical, particularly for use in the prophylactic or curative treatment of a disease of interest. These measures will slow the rate of progress of the disease state and assist the body in reversing the process direction in a natural manner.

It will be appreciated that the pharmaceutical compositions and treatment methods of the invention are useful in fields of human medicine and veterinary medicine. Thus, the subject to be treated is in one aspect a mammal. In another aspect, the mammal is a human.

In jurisdictions that forbid the patenting of methods that are practiced on the human body, the meaning of “administering” of a composition to a human subject shall be restricted to prescribing a controlled substance that a human subject will self-administer by any technique (e.g., orally, inhalation, topical application, injection, insertion, etc.). The broadest reasonable interpretation that is consistent with laws or regulations defining patentable subject matter is intended. In jurisdictions that do not forbid the patenting of methods that are practiced on the human body, the “administering” of compositions includes both methods practiced on the human body and also the foregoing activities.

As described herein, it is believed that the activity of the disclosed compounds may be due, at least in part, to a host immune response. Accordingly, in some embodiments, the disclosed pharmaceutical compositions are suitable for stimulating an immune response in a subject. Thus, in some embodiments the disclosed pharmaceutical compositions further comprise at least one vaccine antigen. Illustrative suitable vaccine antigens include, for example, Smp80, Smp28, Sm14, Sj23, Cathepsin B-like cysteine proteinase, and schistosome glutathione S-transferase P28GST.

### Methods of Use

In some embodiments, the disclosure provides a method of inhibiting AKR1C3 comprising contacting AKR1C3 with a compound or pharmaceutically acceptable salt of formula (A) disclosed herein in an amount effective to inhibit AKR1C3.

Methods of the disclosure can include inhibiting AKR1C3 activity in a subject in need thereof by administration of a compound of formula (A) or a pharmaceutically acceptable salt thereof or a pharmaceutical composition including the same.

The disclosure provides a method of treating, inhibiting, and/or preventing a disease in a subject in need thereof, comprising administering to the subject a therapeutically effective amount of a compound of formula (A) or pharmaceutically acceptable salt.

The disease can be mediated by AKR1C3 activity.

The disease can be cancer. For example, the cancer can be selected from leukemia (e.g., acute lymphoblastic leukemia, acute myeloid leukemia, chronic lymphocytic leukemia, chronic myelogenous leukemia, T-cell acute lymphoblastic leukemia), lymphoma (e.g., Hodgkin lymphoma, non-Hodgkin lymphoma), multiple myeloma, breast cancer, prostate cancer, pancreatic cancer, colon cancer, thyroid cancer, bladder cancer, liver cancer, neuroblastoma, brain cancers (e.g., gliomas, meningiomas, pituitary adenomas, etc.), lung cancer, ovarian cancer, stomach cancer, skin cancer (melanoma), cervical cancer, testicular cancer, kidney cancer, carcinoid tumors, bone cancer, and endometrial cancer. In some embodiments, the cancer is T-cell acute lymphoblastic leukemia. In some embodiments, the cancer is prostate cancer.

The disease can be an inflammatory disease including but not limited to asthma and atopic dermatitis.

The disease can be a gynecological disease including but not limited to polycystic ovary disease and endometriosis.

The disease can be multiple sclerosis.

Methods of the disclosure can include administering the compound of formula A or pharmaceutically acceptable salt thereof in combination with an additional active agent (e.g., anti-cancer agent, anti-inflammatory agent, immune-modulatory agent, and a combination thereof).

The disclosure further provides a method of enhancing or potentiating the effectiveness of an active agent comprising administering the active agent in combination with an effective amount of a compound of formula (A), or pharmaceutically acceptable salt thereof. For example, the active agent can be selected from an anti-cancer drug, including but not limited to navitoclax (ABT-737), daunorubicin, cisplatin, doxorubicin, idarubicin, and dexamethasone.

As used herein, treating refers to alleviating, reducing, and/or stopping of progression of a disease and/or symptoms thereof. As used herein, preventing refers to reducing the chance of contracting or the chance of recurrence of a disease

As used herein, the term “therapeutically effective amount” means an amount of a compound or combination of therapeutically active that ameliorates, attenuates or eliminates one or more symptoms of a particular disease or condition (e.g., parasitic disease), or prevents or delays the onset of one of more symptoms of a particular disease or condition.

As used herein, the terms “subject” and “patient” may be used interchangeably and mean animals, such as dogs, cats, cows, horses, and sheep (e.g., non-human animals) and humans. Particular subjects or patients are mammals (e.g., humans). The terms subject and patient include males and females.

As used herein, the term “pharmaceutically acceptable” means that the referenced substance, such as a compound of the present disclosure, or a formulation containing the compound, or a particular excipient, are safe and suitable for administration to a patient or subject. The term “pharmaceutically acceptable excipient” refers to a medium that does not interfere with the effectiveness of the biological activity of the active ingredient(s) and is not toxic to the host to which it is administered.

In some embodiments, the disclosed methods comprise administering a compound selected from Compound of Formula (A-1), Formula (A′-1), Formula (A-2), Formula (A-3), and Formula (A-4), or a pharmaceutically acceptable salt thereof. In some embodiments, the disclosed methods comprise administering Compound of Formula (A-1) or Formula (A′-1), or a pharmaceutically acceptable salt thereof.

The disclosed methods comprise administering a compound of Formula (A) using any suitable route of administration. Illustrative suitable routes of administration include, for example, parenterally, subcutaneously, orally, topically, pulmonarily, rectally, vaginally, intravenously, intraperitoneally, intrathecally, intracerbrally, epidurally, intramuscularly, intradermally, or intracarotidly.

The compounds described herein can be used to decrease or prevent cancer in human subjects with e.g., prostate cancer. In a particular example, a compound or mixture is administered orally, such as by mixing with distilled water. In another example, a test compound or mixture is administered intravenously, such as in saline or distilled water. In some examples, treatment with test compound may be a single dose or repeated doses. The test compound may be administered about every 6 hours, about every 12 hours, about every 24 hours (daily), about every 48 hours, about every 72 hours, or about weekly. Treatment with repeated doses may continue for a period of time, for example for about 1 week to 12 months, such as about 1 week to about 6 months, or about 2 weeks to about 3 months, or about 1 to 2 months. Administration of a compound may also continue indefinitely. Doses of test compound are from about 0.1 mg/kg to about 400 mg/kg, such as about 1 mg/kg to about 300 mg/kg, about 2 mg/kg to 200 mg/kg, about 10 mg/kg to about 100 mg/kg, about 20 mg/kg to about 75 mg/kg, or about 25 mg/kg to about 50 mg/kg.

The compositions of the present invention may be administered to a patient and may be conveniently formulated for administration with any pharmaceutically acceptable carrier(s).

Uses of the compounds disclosed herein in the preparation of a medicament for treating, inhibiting, and/or preventing parasitic diseases also are provided herein.

### EMBODIMENTS

The disclosure encompasses various embodiments as set forth below.

1. A compound, or a pharmaceutically acceptable salt thereof, having a structure of formula (A):

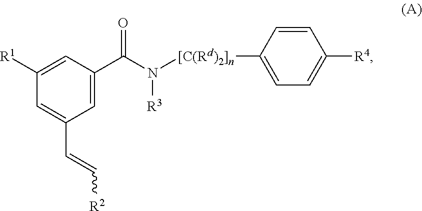

wherein
R¹ is selected from substituted or unsubstituted C₆-C₁₂aryl, substituted or unsubstituted C₂-C₆alkynyl, substituted or unsubstituted C₁-C₆alkyl, C₂-C₆alkenyl, substituted or unsubstituted C₃-C₆alkenyloxy, halo, substituted or unsubstituted C₆-C₁₀aryl-C₁-C₃alkyleneoxy, C₆-C₁₀aryloxy, and substituted or unsubstituted C₅-C₁₀heteroaryl having 1-4 ring heteroatoms selected from N, O, and S;
wherein R¹ is optionally substituted with one or more substituents selected from C₆-C₁₀aryl; 5-15 membered heteroaryl having 1-4 ring heteroatoms selected from N, O, and S; formyl, C₃-C₆cycloalkyl, halo, cycloheteroalkyl-alkylene, hydroxy-C₁-C₃alkylene, methoxymethyl, phenyoxy, cyano, C₁-C₆alkyl, C₁-C₆haloalkyl, and 5-7 membered fused cycloheteroalkyl having 1 to 3 ring heteroatoms selected from N, O, and S;
R² is selected from CO₂R^{a}, CONR^{b}R^{c}, wherein each R^{a}, R^{b}, and R^{c} is independently H or C₁-C₆alkyl;
each R³ and R⁴ is independently H or C₁-C₆alkyl;
each R^{d} is independently H or C₁-C₃alkyl; and
n is 1, 2, or 3.

2. The compound or pharmaceutically acceptable salt of embodiment 1, wherein R¹ is selected from:

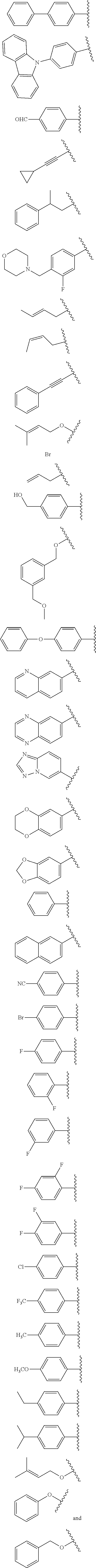

3. The compound or pharmaceutically accountable salt of embodiment 1 or 2, wherein R¹ is selected from:

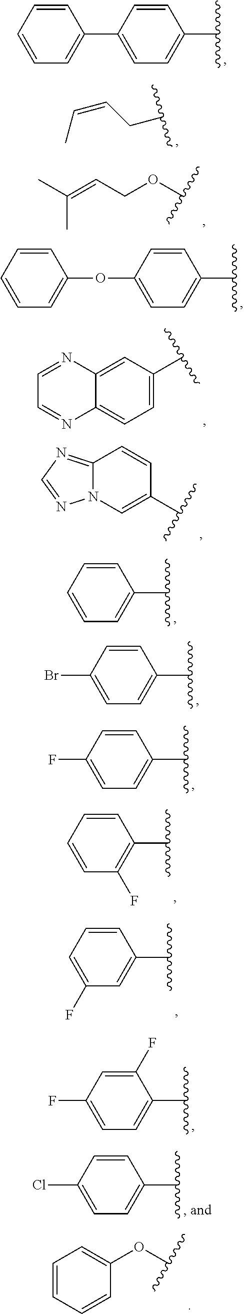

4. The compound or pharmaceutically acceptable salt of any one of embodiments 1-3, wherein R¹ is selected from:

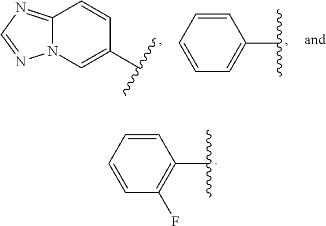 [SMILES: *c1ccc2ncnn2c1 ; *c1ccccc1 ; *c1ccccc1F]

5. The compound or pharmaceutically acceptable salt of any one of embodiments 1-4, wherein R² is selected from —CO₂H, —CO₂CH₃, —CO₂CH₂CH₃, —CO₂(CH₂)₂CH₃, —CO₂C(CH₃)₃, —CONH₂, —CON(CH₃)₂, and —CN,

6. The compound or pharmaceutically acceptable salt of any one of embodiments 1-5, wherein R² is CO₂H.

7. The compound or pharmaceutically acceptable salt of any one of embodiments 1-6, wherein R³ is H.

8. The compound or pharmaceutically acceptable salt of any one of embodiments 1-7, wherein R⁴ is C₁-C₆alkyl.

9. The compound or pharmaceutically acceptable salt of any one of embodiments 1-8, wherein R⁴ is methyl.

10. The compound or pharmaceutically acceptable salt of any one of embodiments 1-9, wherein one or more R^{d} is ethyl.

11. The compound or pharmaceutically acceptable salt of embodiment 1, wherein formula (A) is selected from:

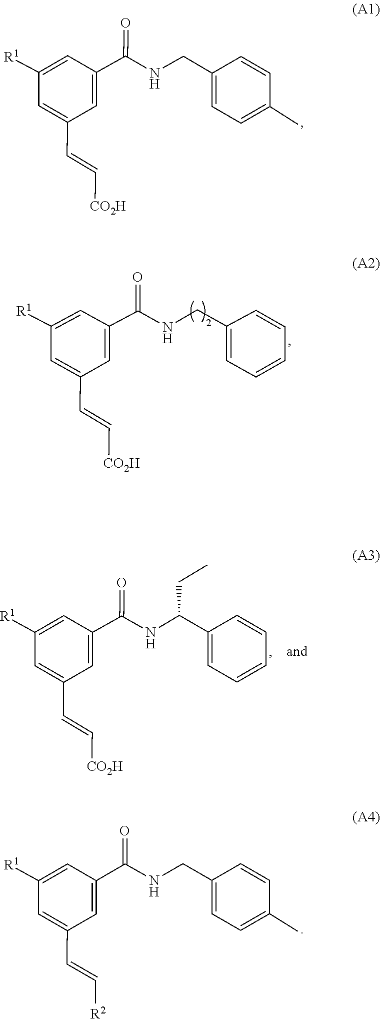

12. The compound or pharmaceutically acceptable salt of embodiment 11, wherein R² of formula A-4 is selected from —CN, —CONH₂, —CON(CH₃)₂, and —CO₂CH₃.

13. The compound or pharmaceutically acceptable salt of any one of embodiments 1-12, wherein the structure of formula (A) is selected from the structures listed in Table 1, Table 3, or Table 4.

14. A pharmaceutical composition comprising the compound of formula (A) or pharmaceutically acceptable salt thereof according to any one of embodiments 1-13 and a pharmaceutically acceptable carrier or excipient.

15. The pharmaceutical composition of embodiment 14, wherein the compound of formula (A) is selected from a compound of formula (A1), formula (A2), formula (A3), and formula (A4).

16. The pharmaceutical composition of embodiment 14 comprising a compound or pharmaceutically acceptable salt thereof listed in Table 1, Table 3, or Table 4.

17. A method of inhibiting AKR1C3 comprising contacting AKR1C3 with a compound or pharmaceutically acceptable salt of formula (A) according to any one of embodiments 1-13 in an amount effective to inhibit AKR1C3.

18. A method of treating and/or preventing a disease in a subject in need thereof, comprising administering to the subject a therapeutically effective amount of a compound of formula (A) or pharmaceutically acceptable salt according to any one of embodiments 1-13.

19. The method of embodiment 18, wherein the disease is cancer.

20. The method of embodiment 19, wherein cancer is selected from leukemia (e.g., acute lymphoblastic leukemia, acute myeloid leukemia, chronic lymphocytic leukemia, chronic myelogenous leukemia, T-cell acute lymphoblastic leukemia), lymphoma (e.g., Hodgkin lymphoma, non-Hodgkin lymphoma), multiple myeloma, breast cancer, prostate cancer, pancreatic cancer, colon cancer, thyroid cancer, bladder cancer, liver cancer, neuroblastoma, brain cancers (e.g., gliomas, meningiomas, pituitary adenomas, etc.), lung cancer, ovarian cancer, stomach cancer, skin cancer (melanoma), cervical cancer, testicular cancer, kidney cancer, carcinoid tumors, bone cancer, and endometrial cancer.

21. The method of embodiment 20, wherein the cancer is T-cell acute lymphoblastic leukemia.

22. The method of embodiment 20, wherein the cancer is prostate cancer.

23. The method of embodiment 18, wherein the disease is an inflammatory disease.

24. The method of embodiment 23, wherein the inflammatory disease is asthma or atopic dermatitis.

25. The method of embodiment 18, wherein the disease is a gynecological disease.

26. The method of embodiment 25, wherein gynecological disease is polycystic ovary disease or endometriosis.

27. The method of embodiment 18, wherein the disease is multiple sclerosis.

28. The method of any one of embodiments 18-27, wherein the disease is mediated by AKR1C3 activity.

29. The method of any one of embodiments 18-28, comprising administering the compound of formula A or pharmaceutically acceptable salt thereof in combination with an additional active agent.

30. The method of embodiment 29, wherein the additional active agent is selected from an anti-cancer agent, anti-inflammatory agent, immune-modulatory agent, and a combination thereof.

31. A method of enhancing or potentiating the effectiveness of an active agent comprising administering the active agent in combination with an effective amount of a compound or pharmaceutically acceptable salt according to any one of embodiments 1-13.

32. The method of embodiment 2931, wherein the active agent is selected from an anti-cancer drug, including but not limited to navitoclax (ABT-737), daunorubicin, cisplatin, doxorubicin, idarubicin, and dexamethasone.

33. The composition of embodiment 14 for use in inhibiting AKR1C3.

34. The composition of embodiment 14 for use in treating and/or preventing a disease in a subject in need thereof.

35. The composition for use of embodiment 34, wherein the disease is mediated by AKR1C3 activity.

36. The composition for use of embodiment 34, wherein the disease is selected from cancer, an inflammatory disease, a gynecological disease, and multiple sclerosis.

37. The composition for use of embodiment 34, wherein the composition comprises a compound selected from a compound of formula (A1), (A2), (A3), and (A4), or a pharmaceutical salt thereof.

38. The composition for use of embodiment 34, wherein the composition comprises a compound selected from the compounds listed in Table 1, Table 3, and Table 4, or a pharmaceutical salt thereof.

39. Use of the composition of embodiment 14 for treating and/or preventing a disease in a subject in need thereof.

40. The use of the composition of embodiment 39, wherein the disease is selected from cancer, an inflammatory disease, a gynecological disease, and multiple sclerosis.

41. The use of the composition of embodiment 37, wherein the composition comprises a compound selected from a compound of formula (A1), (A2), (A3), and (A4), or a pharmaceutical salt thereof.

42. The use of the composition of embodiment 37, wherein the composition comprises a compound listed in Table 1, Table 3, or Table 4, or a pharmaceutical salt thereof.

The foregoing summary is not intended to define every aspect of the disclosure, and additional aspects are described in other sections of the disclosure. The entire document is intended to be related as a unified disclosure, and it should be understood that all combinations of features described herein are contemplated, even if the combination of features are not found together in the same sentence, or paragraph, or section of this document.

In addition to the foregoing, the disclosure includes, as an additional aspect, all embodiments of the disclosure narrower in scope in any way than the variations specifically mentioned above. With respect to aspects of the disclosure described or claimed with “a” or “an,” these terms mean “one or more” unless context unambiguously requires a more restricted meaning. With respect to elements described as one or more within a set, all combinations within the set are contemplated as combination inventions. If aspects of the disclosure are described as “comprising” a feature, embodiments also are contemplated “consisting of” or “consisting essentially of” the feature.

Aspects of the disclosure described as methods of treatment should also be understood to include first or subsequent “medical use” aspects of the disclosure or “Swiss use” of compositions for the manufacture of a medicament for treatment of the same disease or condition.

Multiple embodiments are contemplated for combinations described herein. For example, some aspects of the disclosure that are described as a method of treatment (or medical use) combining two or more compounds or agents, whether administered separately (sequentially or simultaneously) or in combination (co-formulated or mixed). For each aspect described in this manner, the disclosure further includes a composition comprising the two or more compounds or agents co-formulated or in admixture with each other; and the disclosure further includes a kit or unit dose containing the two or more compounds/agents packaged together, but not in admixture. Optionally, such compositions, kits or doses further include one or more carriers in admixture with one or both agents or co-packaged for formulation prior to administration to a subject. The reverse also is true: some aspects of the disclosure are described herein as compositions useful for therapy and containing two or more therapeutic agents. Equivalent methods and uses are specifically contemplated.

Although the applicant(s) invented the full scope of the claims appended hereto, the claims appended hereto are not intended to encompass within their scope the prior art. Therefore, in the event that statutory or judicially recognized prior art within the scope of a claim is brought to the attention of the applicants by a Patent Office or other entity or individual, the applicant(s) reserve the right to exercise amendment rights under applicable patent laws to redefine the subject matter of such a claim to specifically exclude such prior art or obvious variations of statutory prior art from the scope of such a claim. Variations of the disclosure defined by such amended claims also are intended as aspects of the invention. Additional features and variations of the invention will be apparent to those skilled in the art from the entirety of this application, and all such features are intended as aspects of the disclosure.

The disclosure herein will be understood more readily by reference to the following examples, below.

### EXAMPLES

The following examples are provided for illustration and are not intended to limit the scope of the disclosure.

The following abbreviations are used herein: ADME refers to absorption, distribution, metabolism and excretion; AKR1C3 refers to aldo-keto reductase 1C3; AR refers to androgen receptor; ARv7 refers to androgen receptor splice variant 7; NH₄OH refers to ammonium hydroxide; CRPC refers to castration-resistant prostate cancer; Cs₂CO₃ refers to cesium carbonate; DHT refers to dihydrotestosterone; DMF refers to N,N-dimethylformamide; DMSO refers to dimethyl sulfoxide; EDC refers to (1-ethyl-3-(3-dimethylaminopropyl)carbodiimide hydrochloride); ENZ refers to enzalutamide; GI refers to gastrointestinal: 17β-HSD refers to 17β-hydroxysteroid dehydrogenase; HOBt refers to 1-hydroxybenzotriazole: 7-HC refers to 7-hydroxycoumarin; HLM refers to human liver microsomes; MeOH refers to methanol; MLM refers to mouse liver microsomes; NADP+ refers to adenine dinucleotide phosphate; NSAID refers to nonsteroidal anti-inflammatory drug; PCa refers to prostate cancer; PG refers to prostaglandin; PSA refers to prostate specific antigen; Pd(OAc)₂ refers to palladium(II) acetate; K₂CO₃ refers to potassium carbonate; PPh₃ refers to triphenylphosphine; Pd(dppf)Cl₂—CH₂Cl₂ refers to [1,1′-bis(diphenylphosphino)ferrocene]dichloropalladium(II), complex with dichloromethane; SGF refers to simulated gastric fluid; SIF refers to simulated intestinal fluid; NaOH refers to sodium hydroxide; SP refers to sub-pocket; Et₃N refers to triethylamine; T refers to testosterone; THF refers to tetrahydrofuran; SOCl₂ refers to thionyl chloride.

General Chemistry. All reactions were carried out in oven- or flame-dried glassware under a nitrogen atmosphere unless otherwise noted. Reaction progress was monitored by thin-layer chromatography carried out on silica gel plates (2.5 cm×7.5 cm, 200 μm thick, 60 F254) and visualized using UV (254 nm) or by potassium permanganate and/or phosphomolybdic acid solution and/or ninhydrin as an indicator. Flash column chromatography was performed with silica gel (40-63 μm, 60 Å) using the mobile phase indicated or on a Biotage Selekt (Rf 200 UV/vis). Solvents and reagents were purchased from Fisher Scientific, Sigma-Aldrich was used without further purification, except as indicated.

¹H and ¹³C NMR spectra were recorded on Bruker 600 MHZ, 500 MHz or 400 MHZ spectrometers. The chemical shifts of ¹H NMR are reported in parts per million (ppm) relative to the internal standard tetramethylsilane or residual solvent peak. ¹³C NMR chemical shifts are reported in ppm with the solvents (CDCl₃: 77.23 ppm, CD₃OD: 49.15 ppm, DMSO-d6: 39.51 ppm). Multiplicities are indicated by s (singlet), d (doublet), dd (doublet of doublets), t (triplet), q (quartet), m (multiplet), and br (broad). Chemical shifts (δ) are reported in parts per million (ppm) and coupling constants (J) are reported in hertz. High-resolution mass spectra (HRMS) were recorded with an Agilent 6230 LC/TOF spectrometer using an ESI source coupled to an Agilent Infinity 1260 system running in reverse phase with a ZORBAX RRHT Extend-C18 (80 Å, 2.1×50 mm, 1.8 μm) column using solvent A (water with 0.1% Formic acid), solvent B (acetonitrile with 0.1% Formic acid), and a flow rate of 0.6 mL/min starting a mixture of 95% A and 5% B. Solvent B is gradually increased to 95% at 5 min, held at 95% until 6 min, then gradually ramped back down to 5% at 8.0 min. The purity analysis of final compounds were determined ≥95% pure (except 16a, which also contains reverse prenylated compound) using a Waters ACQUITY ultra-performance liquid chromatography (UPLC) H-Class System with TUV (254 nm) detector and Empower 2 software (Milford, MA, USA) using an Agilent Eclipse plus C18 5μ column (4.6×150 mm). Chromatography was performed using solvent A (water with 0.1% Trifluoroacetic acid), solvent B (methanol with 0.1% Trifluoroacetic acid), and a flow rate of 1.0 mL/min for 20 min. with an isocratic system (20:80, A:B) (traces and purity analysis can be viewed in the Supporting Information).

In silico modelling: Molecular docking studies of AKR1C3 inhibitors to AKR1C3.NADP+ complex was carried out using Schrödinger Maestro 13.3. The AKR1C3 crystal structure was retrieved from RCSB protein data bank (PDB:3UG8) and processed by default with the Protein Preparation Workflow panel (Schrödinger, 2022-3 version), and the prepared protein-ligand complex was defined as the binding site. The size of the docking Glide grid box was 20 Å×20 Å×20 Å. Based on the OPLS4 force field, the grid of AKR1C3 crystal structure was generated. As per default options, the ligands were prepared with LigPrep tool, and docked using extra precision (XP) mode without constrained binding.

Liquid chromatography and mass spectrometry (LCMS/MS): The ultra-performance liquid chromatography mass spectrometry system (UPLC-MS/MS) consisted of a Shimadzu 8060NX mass spectrometer and Nexera Series UPLC (Shimadzu Scientific Instruments, Columbia, MD). Analyte separation was achieved utilizing an Acquity UPLC® BEH column (C18, 2.1×100 mm, 1.7 μm) equipped with an Acquity UPLC C18 guard column (Waters, Inc. Milford MA). The mobile phase consisted of water containing 0.1% formic acid (mobile phase A) and methanol (mobile phase B) at a flow rate of 0.25 mL/min operated at room temperature. The total run time was set to last 7.5 min with a gradient elution as follows: 35% B, increasing to 95% B over 3.5 minutes, then held constant for 3.0 minutes, and finally brought back to the initial condition of 35% B in 0.20 minutes followed by 1-minute re-equilibration. The injection volume (2 μL) was consistent for all samples. The auto-sampler chamber was maintained at 4° C. throughout the analysis.

The MS/MS system was operated at unit resolution in the multiple reaction monitoring (MRM) in positive ESI mode, using precursor ion>product ion combinations of 427.20>105.10 for A1-r and 413.15>265.10 m/z for 4r. The mass spectrometer source settings were optimized to the following: nebulizer gas: 2.0 L/min; heating gas: 10 L/min; drying gas: 10 L/min; interface temperature: 300° C.; desolvation line temperature: 250° C.; heat block temperature: 400° C. Data acquisition and quantitation were performed using LabSolutions software Ver.5.99 (Shimadzu Scientific Inc, Columbia, MD).

Plasma sample preparation: Plasma samples were prepared by spiking 5 μL of the appropriate calibration (CC) and quality (QCs) control working stock into a 45 μL blank mouse plasma. The concentration of the CC ranged from 0.2-1000 ng/mL with the final concentrations of 0.2, 0.5, 1, 5, 10, 50, 100, 500, and 1000 ng/mL. A simple protein precipitation technique using Phree 96 well phospholipid elimination plate (Phenomenex Inc, Torrance CA.) was utilized to isolate analyte from the plasma matrix. The CC, QCs and study plasma samples were added to Phree 96 well plate and spiked with 10 μL of IS a working solution. The precipitation of the matrix proteins was carried out using 300 μL of ice-cold acetonitrile. The plate was again vortexed on mixmate at 950 rpm for 2 min followed by applied 5 psi positive pressure for 10 min (Resprep VM-96 Vacuum Manifold for 96-Well Plates, Catalog #25858) (Restek; Bellefonte, PA) to collect the supernatant. Two microliters of the reconstituted sample was injected into LC-MS/MS for analysis.

In vitro stability studies: Gastrointestinal (GI) fluid stability studies were performed in simulated gastric fluid (SGF) and simulated intestinal fluid (SIF). All the required media were prepared according to the USP guidelines. Mouse plasma stability were performed for neat-spiked and plasma spiked samples (pre-extraction) at 37° C. for 4 h. In addition to the analyte stability in matrix, at 48 h stability of the extracted samples was performed at 4° C. in the autosampler.

In vitro metabolic stability in liver microsomes: Metabolic stability was assessed using mouse and human liver microsomes, (XenoTech, LLC, Lenexa, KS). Incubation of A1-r and 4r with the microsome fractions was performed in triplicate utilizing a concentration of 1 μg/mL as previously described. Serial samples (40 μL) were collected at selected time intervals and quenched with 300 μL of acetonitrile and then spiked with 10 μL of IS (0.5 μg/mL). All the samples were vortexed and centrifuged at 13,000×g for 15 min, and the supernatant was collected and transferred to an autosampler vial and injected (2 μL) onto the LC-MS/MS system. Testosterone, 7-HC and diclofenac were used as positive controls to ensure that the microsomes and incubation conditions were appropriate to conduct metabolism studies.

In vivo pharmacokinetic studies: Animal studies were approved by the University of Nebraska Medical Center (UNMC) Institutional Animal Care and Use Committee (IACUC protocol number 17-046-06-FC). BALB/c mice were purchased from Charles River Labs. Pharmacokinetic (PK) studies of A1-r and 4r were conducted in BALB/c mice. Animals were housed in the University of Nebraska Medical Center animal facility, for at least 7 days prior to the experiments, in order to acclimatize the animals to the laboratory conditions, at a temperature of 23-24° C., relative humidity of 40-70% and 12/12 h light/dark cycles with free access to food and water. The dosing solution was made of DMSO-Polyethylene glycol 400 (PEG400)-Propylene glycol (PG)-EtOH-Cremophore-PBS (2/20/10/10/5/53% v/v). Compound A1-r or 4r (10 mg/kg) was administered separately by oral gavage. After dosing, approximately 50 μL of blood was collected from the maxillary vein at 5, 15, 30 minutes and 1, 2, 7, and 24 hr (5 mice/group/per time point). A total of three blood samples were collected, with the third blood sample a terminal collection. Plasma was separated by centrifugation at 4,000×g at 4° C. for 10 minutes. The collected plasma samples were stored at −80° C. until analysis. A non-compartmental analysis (NCA) was performed to estimate PK parameters using Phoenix WinNonlin® 8.2 (Certara Corporation, Mountain View, CA, USA).

Tumor xenograft study: All animal experiments were carried out according to approved Institutional Animal Care and Use Committee protocols at the University of Texas Southwestern Medical Center (Dallas, TX). Five-week-old female NSG mice (low circulating testosterone) were implanted with 3×10⁶ 22 Rv1 cells (ATCC, mycoplasma negative) in matrigel. When tumor volume reached approximately 125 mm³ (day 11), mice were randomly divided into three groups of six mice. One group was treated with vehicle control PO (0.5% Methocel A4M+0.1% Tween 80)+vehicle IP (10% DMSO/10% cremophor EL/80% D5W). One group was treated with vehicle PO+IP 25 mg/kg A1-r in 10% DMSO/10% cremophor EL/80% D5W. One group was treated with vehicle PO+IP 50 mg/kg 4r in 10% DMSO/10% cremophor EL/80% D5W. Dosing was continued for a total of 26 days. Tumor volumes were measured twice a week with Vernier calipers, and tumor volume was calculated as (L×W²)×3.14)/6 as previously reported. 2-3 hours after a final IP dose, animals were humanely euthanized, and tumors were collected, weighed, and frozen in liquid nitrogen after taking pictures.

Synthesis. Compounds of the disclosure were prepared using the synthetic routes depicted in Schemes 1-5.

Scheme 1 depicts an illustrative synthesis of compounds of formula (A-1).

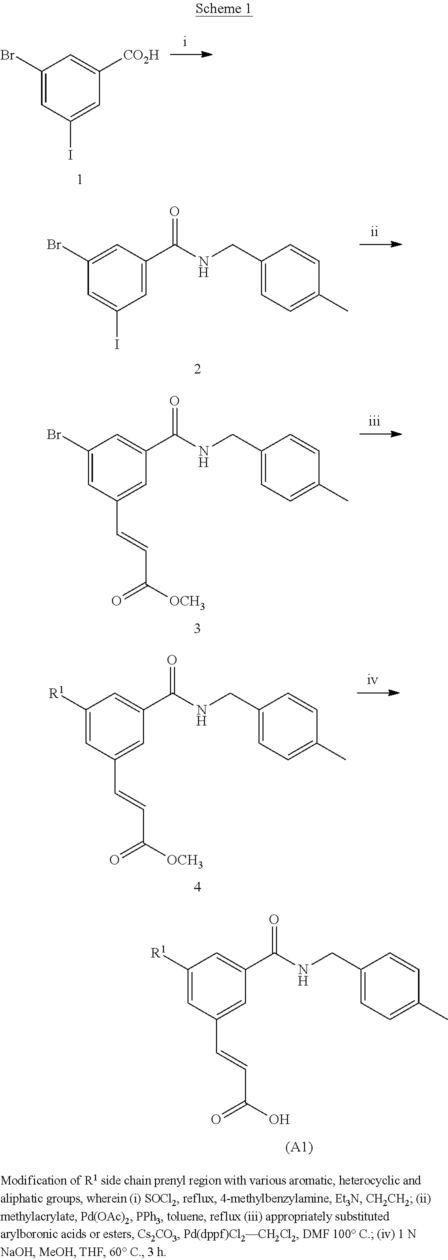

As depicted herein, compounds of formula (A1) can be obtained from the corresponding compound of formula 4.

General procedure for the synthesis of methyl esters (4a-bb shown in Table 1). To a stirred solution of aryl bromide 3 (0.20 g, 0.50 mmol) and appropriately substituted arylboronic acids or esters (0.21 g, 1.0 mmol) in anhydrous DMF (10 mL) were added Pd(dppf)Cl₂·CH₂Cl₂ (40 mg, 0.05 mmol) and CS₂CO₃ (0.50 g, 1.5 mmol). The mixture was stirred at 100° C. for 12 under N₂ atmosphere. After cooled to room temperature, the reaction mixture was filtered through celite and washed with EtOAc (50 mL). The filtrate was washed with 1 N HCl (30 mL) and brine 50 (mL). The aqueous layer was extracted with EtOAc (2×50 mL), and the combined organic layers were dried over Na₂SO₄, filtered, and evaporated to dryness under reduced pressure. The residue was purified by silica gel column chromatography or Biotage Selekt flash column using EtOAc/Hexanes as eluent to afford the pure esters.

General procedure for the synthesis of target compounds (A1-a to A1-bb shown in Table 1). To a stirred solution of methyl esters 4a-bb (0.2 g, 0.42 mmol) in a mixture of THF/MeOH (10 mL, 4:1) was added aqueous 1 N NaOH (1.3 mL, 1.3 mmol). The mixture was stirred at 60° C. for 3 h. The solvent was evaporated in vacuo and the pH of the reaction mixture was adjusted to 2-4 with 1 N HCl. The mixture was extracted with CH₂Cl₂ (3×50 mL) and washed with brine (50 mL). The combined organic layers were dried over anhydrous Na₂SO₄, filtered, and evaporated to dryness under reduced pressure. The residue was purified by silica gel column chromatography or Biotage Selekt flash column using CH₂Cl₂/MeOH as eluent to afford the pure acids.

In an illustrative synthesis, 3-bromo-5-iodobenzoic acid (1) was treated with SOCl₂ under reflux afforded acid chloride, which was subsequently coupled with 4-methyl benzylamine in the presence of Et₃N at room temperature to yield amide 2 in excellent yields. The amide underwent selective Heck coupling with methyl acrylate in the presence of Pd(OAc)₂ and PPh₃ to afford methyl esters of type 3. Suzuki coupling with various functionalized aryl boronic acids or esters mediated by Pd(dppf)Cl₂—CH₂Cl₂ and Cs₂CO₃ afforded access to the corresponding aryl-substituted esters (4a-4bb) in good yields. The methyl ester was hydrolyzed with aqueous 1 N NaOH in a mixture of THE/MeOH (4:1) at 60° C. to yield the target trans-cinnamic acids (A1-a-A1-bb).

Scheme 2 depicts an illustrative synthesis of compounds of formula (A) wherein R¹ is a O-prenyl group and R² is C₁-C₆alkyl or hydrogen (e.g., 9 and 10). In an illustrative synthesis, 3-bromo-5-hydroxybenzoic acid (6) underwent a sequence of Heck coupling conditions as described in Scheme 1, followed by amide formation with 4-methylbenzylamine in the presence of EDC, HOBt, and Et₃N at room temperature. The resultant phenol 8 was O-prenylated with prenyl bromide, and a base. The resulting methyl ester (9) was hydrolysed with 1 N NaOH in the same reaction condition as described in Scheme 1 to afford the target acid (10) in good yield.

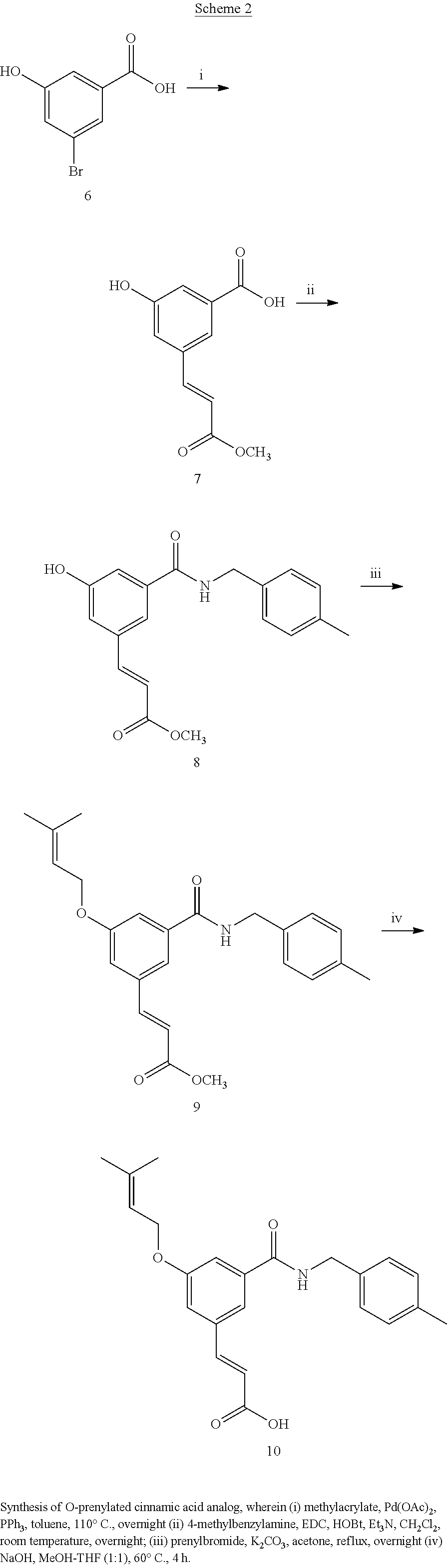

Scheme 3 depicts an illustrative synthesis of compounds of formula (A) wherein R¹ is phenoxyphenyl and R² is amide. In an illustrative synthesis, (E)-3-(3-bromo-5-((4-methylbenzyl)carbamoyl)phenyl)acrylic acid 11 was obtained by hydrolysis of the methyl ester with 1 N NaOH. The acid was converted to carboxamide by reaction with SOCl₂ to afford the corresponding acid chloride which was then treated with 30% aqueous NH₄OH to afford bromide 12. The phenoxyphenyl substituted analogue 13 was accessed by applying the same reaction conditions to starting material A1-n (Scheme 3).

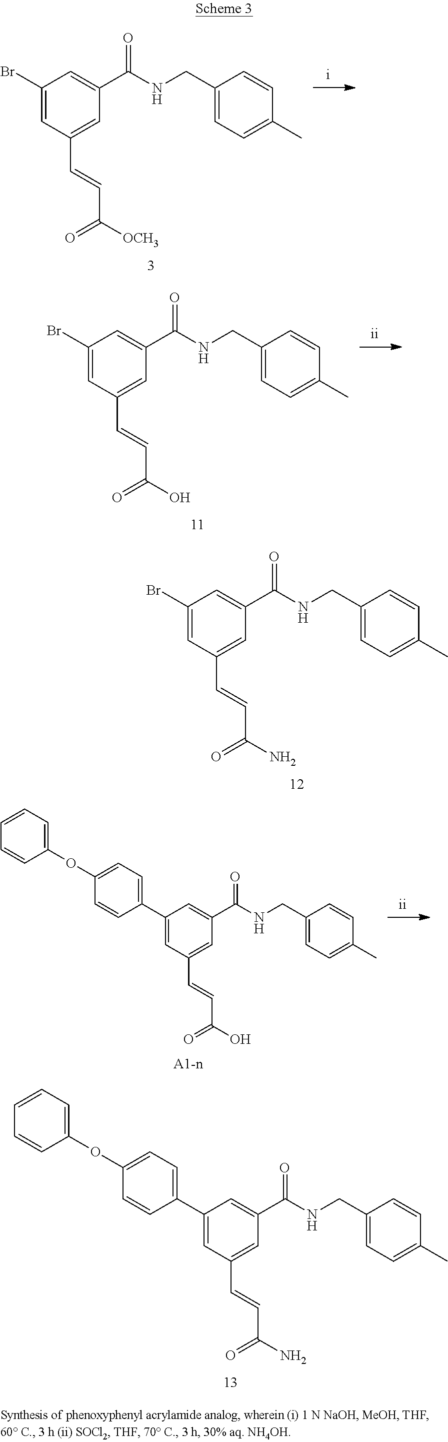 [SMILES: COC(=O)/C=C/c1cc(Br)cc(C(=O)NCc2ccc(C)cc2)c1 ; Cc1ccc(CNC(=O)c2cc(Br)cc(/C=C/C(=O)O)c2)cc1 ; Cc1ccc(CNC(=O)c2cc(Br)cc(/C=C/C(N)=O)c2)cc1 ; Cc1ccc(CNC(=O)c2cc(/C=C/C(=O)O)cc(-c3ccc(Oc4ccccc4)cc3)c2)cc1 ; Cc1ccc(CNC(=O)c2cc(/C=C/C(N)=O)cc(-c3ccc(Oc4ccccc4)cc3)c2)cc1]

Scheme 4 depicts an illustrative synthesis of compounds of formula (A) wherein R¹ is phenoxyphenyl or prenyl and R² is nitrile. In an illustrative synthesis, the previously obtained intermediate 3-bromo-5-iodo-N-(4-methylbenzyl)benzamide (2), underwent selective Heck coupling with N,N-dimethylacrylamide, or acrylonitrile at the iodo position, in the same reaction conditions as described in Scheme 1. Bromides 14 and 15 were then exposed to Suzuki conditions with (4-phenoxyphenyl)boronic acid or (3-methylbut-2-en-1-yl)boronic acid to yield the target compounds 16a and 16b.

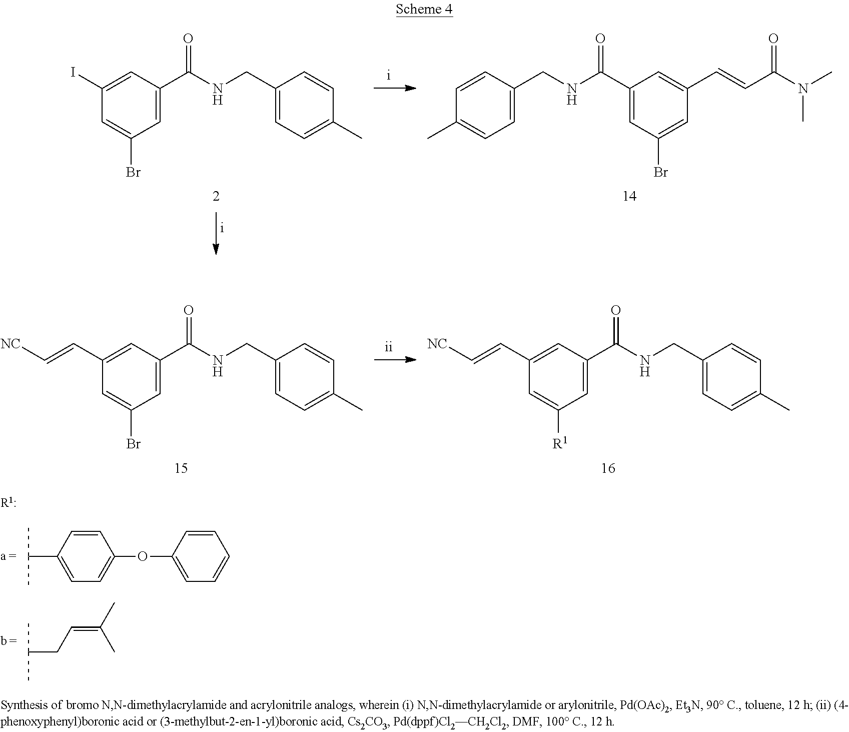

Scheme 5 depicts an illustrative synthesis of compounds of formula (A-3). In an illustrative synthesis,

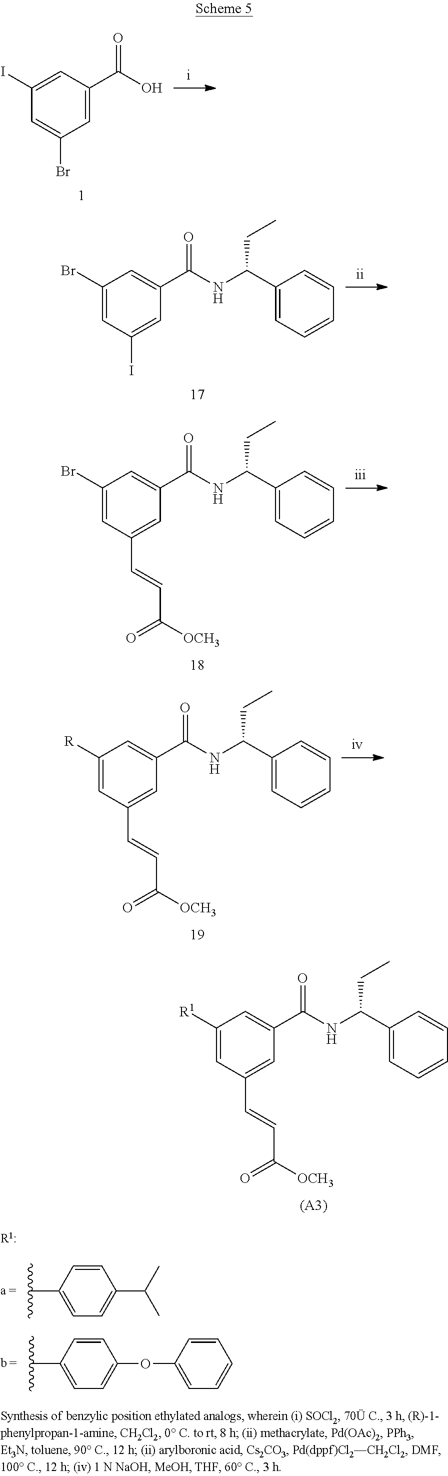

Methyl (E)-3-(5-((4-methylbenzyl)carbamoyl)-[1,1′-biphenyl]-3-yl)acrylate (4a). White solid (120 mg, Yield 60%). ¹H NMR (400 MHZ, CDCl₃): δ 8.01 (1H, s, ArCH), 7.91 (1H, s, ArCH), 7.79 (1H, s, ArCH), 7.72 (1H, d, J=16.0 Hz, CH), 7.57 (2H, d, J=7.2 Hz, ArCH), 7.47-7.39 (3H, m, ArCH), 7.26 (2H, d, J=8.0 Hz, ArCH), 7.16 (2H, d, J=8.0 Hz, ArCH), 6.93 (1H, t, J=5.2 Hz, NH), 6.52 (1H, d, J=16.0 Hz, CH), 4.61 (2H, d, J=5.6 Hz, CH₂), 3.80 (3H, s, OCH₃), 2.35 (3H, s, CH₃). ¹³C NMR (100 MHZ, CDCl₃): δ 167.10, 166.77, 143.74, 142.41, 139.46, 137.37, 135.85, 135.28, 135.00, 129.52, 129.46, 128.99, 128.14, 127.99, 127.45, 127.16, 125.20, 119.29, 51.82, 44.05, 21.12.

(E)-3-(5-((4-Methylbenzyl)carbamoyl)-[1,1′-biphenyl]-3-yl)acrylic acid (A1-a). White solid (68 mg, Yield 71%). ¹H NMR (400 MHZ, DMSO-d₆): δ 9.18 (1H, t, J=5.6 Hz, NH), 8.20 (2H, s, ArCH), 8.15 (1H, s, ArCH), 7.81 (2H, d, J=7.2 Hz, ArCH), 7.72 (1H, d, J=16.0 Hz, CH), 7.52 (2H, t, J=7.6 Hz, ArCH), 7.43 (1H, t, J=7.2 Hz, ArCH), 7.25 (2H, d, J=8.0 Hz, ArCH), 7.15 (2H, d, J=8.0 Hz), 6.74 (1H, d, J=16.0 Hz, CH), 4.49 (2H, d, J=5.6 Hz, CH₂), 2.29 (3H, s, CH₃). ¹³C NMR (100 MHZ, DMSO-d₆): δ 168.01, 165.90, 143.45, 141.43, 139.40, 137.03, 136.88, 136.35, 135.95, 135.74, 129.76, 129.46, 129.34, 128.53, 127.81, 127.46, 125.79, 121.45, 42.99, 21.14. HRMS-ESI (m/z): [M+H]⁺ calcd for C₂₄H₂₂NO₃, 372.1594; found, 372.1595.

Methyl (E)-3-(4′-bromo-5-((4-methylbenzyl)carbamoyl)-[1,1′-biphenyl]-3-yl)acrylate (4b). White solid (54 mg, Yield 48%). ¹H NMR (500 MHZ, CDCl₃): δ 7.95 (1H, s, ArCH), 7.88 (1H, s, ArCH), 7.74 (1H, s, ArCH), 7.70 (1H, d, J=16.0 Hz, CH), 7.56 (2H, d, J=8.0 Hz, ArCH), 7.43 (2H, d, J=8.0 Hz, ArCH), 7.25 (2H, d, J=8.0 Hz, ArCH), 7.16 (2H, d, J=8.0 Hz, ArCH), 6.64 (1H, t, J=5.5 Hz, NH), 6.52 (1H, d, J=16.0 Hz, CH), 4.61 (2H, d, J=5.5 Hz, CH₂), 3.80 (3H, s, OCH₃), 2.24 (3H, s, CH₃). ¹³C NMR (100 MHZ, CDCl₃): δ 166.99, 166.56, 143.44, 141.19, 138.32, 137.46, 135.99, 135.46, 134.90, 132.13, 129.49, 129.23, 128.70, 128.01, 127.26, 125.36, 122.55, 119.56, 51.88, 44.10, 21.13.

(E)-3-(4′-Bromo-5-((4-methylbenzyl)carbamoyl)-[1,1′-biphenyl]-3-yl)acrylic acid (A1-b). White solid (28 mg, Yield 73%). ¹H NMR (400 MHZ, DMSO-d₆): δ 9.18 (1H, t, J=5.6 Hz, NH), 8.21-8.17 (3H, m, ArCH), 7.79 (2H, d, J=8.4 Hz, ArCH), 7.72 (1H, d, J=16.0 Hz, CH), 7.70 (2H, d, J=7.6 Hz, ArCH), 7.25 (2H, d, J=7.6 Hz, ArCH), 7.15 (2H, d, J=7.6 Hz, ArCH), 6.76 (1H, d, J=16.0 Hz, CH), 4.50 (2H, d, J=5.6 Hz, CH₂), 2.29 (3H, s, CH₃). ¹³C NMR (100 MHz, DMSO-d₆): δ 167.92, 165.78, 143.48, 140.09, 138.54, 136.84, 136.36, 136.06, 135.82, 132.34, 129.60, 129.56, 129.35, 127.81, 127.28, 126.26, 122.10, 121.39, 43.01, 21.15. HRMS-ESI (m/z): [M+H]⁺ calcd for C₂₄H₂₁BrNO₃, 450.0699; found, 450.0673.

Methyl (E)-3-(4′-chloro-5-((4-methylbenzyl)carbamoyl)-[1,1′-biphenyl]-3-yl)acrylate (4c). White solid (125 mg, Yield 58%). ¹H NMR (400 MHZ, CDCl₃): δ 7.97 (1H, s, ArCH), 7.89 (1H, s, ArCH), 7.75 (1H, s, ArCH), 7.70 (1H, d, J=16.0 Hz, CH), 7.50 (2H, d, J=8.0 Hz, ArCH), 7.42 (2H, d, J=7.6 Hz, ArCH), 7.26 (2H, d, J=7.6 Hz, ArCH), 7.17 (2H, d, J=7.6 Hz, ArCH), 6.85 (1H, br s, NH), 6.52 (1H, d, J=16.0 Hz, CH), 4.61 (2H, d, J=4.8 Hz, CH₂), 3.81 (3H, s, OCH₃), 2.35 (3H, s, CH₃). ¹³C NMR (100 MHZ, CDCl₃): δ 166.99, 166.58, 143.45, 141.19, 137.86, 137.47, 135.97, 135.44, 134.89, 134.37, 129.49, 129.28, 129.18, 128.40, 128.02, 127.30, 125.31, 119.55, 51.87, 44.10, 21.12.

(E)-3-(4′-Chloro-5-((4-methylbenzyl)carbamoyl)-[1,1′-biphenyl]-3-yl)acrylic acid (A1-c). White solid (57 mg, Yield 59%). ¹H NMR (400 MHZ, DMSO-d₆): δ 9.22 (1H, t, J=5.6 Hz, NH), 8.22 (2H, s, ArCH), 8.15 (1H, s, ArCH), 7.86 (2H, d, J=8.4 Hz, ArCH), 7.72 (1H, d, J=16.0 Hz, CH), 7.55 (2H, d, J=8.4 Hz, ArCH), 7.25 (2H, d, J=7.6 Hz, ArCH), 7.14 (2H, d, J=7.6 Hz, ArCH), 6.77 (1H, d, J=16.0 Hz, CH), 4.50 (2H, d, J=5.6 Hz, CH₂), 2.27 (3H, s, CH₃). ¹³C NMR (100 MHZ, DMSO-d₆): δ 168.16, 165.82, 143.18, 140.04, 138.20, 136.87, 136.34, 136.03, 135.90, 133.47, 129.60, 129.39, 129.33, 129.22, 127.82, 127.28, 126.18, 121.81, 43.03, 21.13. HRMS-ESI (m/z): [M+H]⁺ calcd for C₂₄H₂₁ClNO₃, 406.1204; found, 406.1205.

Methyl (E)-3-(4′-fluoro-5-((4-methylbenzyl)carbamoyl)-[1,1′-biphenyl]-3-yl)acrylate (4d). White solid (160 mg, Yield 80%). ¹H NMR (500 MHZ, CDCl₃): δ 7.94 (1H, s, ArCH), 7.86 (1H, s, ArCH), 7.69 (1H, s, ArCH), 7.66 (1H, d, J=16.0 Hz, CH), 7.50-7.48 (2H, m, ArCH), 7.22 (2H, d, J=7.5 Hz, ArCH), 7.13-7.08 (4H, m, ArCH), 6.99 (1H, t, J=5.0 Hz, NH), 6.48 (1H, d, J=16.0 Hz, CH), 4.56 (2H, d, J=5.0 Hz, CH₂), 3.77 (3H, s, OCH₃), 2.31 (3H, s, CH₃). ¹³C NMR (125 MHZ, CDCl₃): δ 167.03, 166.68, 162.88 (d, J=246.3 Hz), 143.56, 141.36, 137.38, 135.90, 135.56 (d, J=2.5 Hz), 135.33, 134.96, 129.45, 129.30, 128.78 (d, J=7.5 Hz), 127.97, 127.37, 125.07, 119.40, 115.9 (d, J=21.3 Hz), 51.83, 44.04, 21.10.

(E)-3-(4′-Fluoro-5-((4-methylbenzyl)carbamoyl)-[1,1′-biphenyl]-3-yl)acrylic acid (A1-d). White solid (73 mg, Yield 76%). ¹H NMR (500 MHZ, DMSO-d₆): δ 9.22 (1H, t, J=6.0 Hz, NH), 8.20 (2H, s, ArCH), 8.14 (1H, s, ArCH), 7.88-7.85 (2H, m, ArCH), 7.72 (1H, d, J=16.0 Hz, CH), 7.34 (2H, d, J=9.0 Hz, ArCH), 7.25 (2H, d, J=8.0 Hz, ArCH), 7.15 (2H, d, J=8.0 Hz, ArCH), 6.76 (1H, d, J=16.0 Hz, CH), 4.49 (2H, d, J=6.0 Hz, CH₂), 2.28 (3H, s, CH₃). ¹³C NMR (125 MHZ, DMSO-d₆): δ 167.93, 165.85, 162.71 (d, J=243.7 Hz), 143.58, 140.36, 136.88, 136.34, 135.98 135.85 (d, J=1.3 Hz), 135.70, 129.65, 129.58, 129.55 (d, J=7.5 Hz), 129.33, 127.82, 127.38, 125.88, 121.26, 116.26 (d, J=21.6 Hz), 42.99, 21.14. ¹⁹F NMR (376 MHZ, DMSO-d₆): −114.58. HRMS-ESI (m/z): [M+H]⁺ calcd for C₂₄H₂₁FNO₃, 390.1500; found, 390.1511.

Methyl (E)-3-(2′-fluoro-5-((4-methylbenzyl)carbamoyl)-[1,1′-biphenyl]-3-yl)acrylate (4e). White solid (165 mg, Yield 79%). ¹H NMR (400 MHZ, CDCl₃): δ 7.96 (2H, s, ArCH), 7.76 (1H, s, ArCH), 7.68 (1H, d, J=16.0 Hz, CH), 7.32-7.43 (2H, m, ArCH), 7.24-7.18 (4H, m, ArCH), 7.15-7.11 (3H, m, 2 ArCH, 1 NH, Overlapped), 6.49 (1H, d, J=16.0 Hz, CH), 4.58 (2H, d, J=5.6 Hz, CH₂), 3.78 (3H, s, OCH₃), 2.32 (3H, s, CH₃). ¹³C NMR (100 MHZ, CDCl₃): δ 167.08, 166.64, 159.64 (d, J=247.0 Hz), 143.60, 137.26, 136.91, 135.60, 135.01 (d, J=5.0 Hz), 131.44 (d, J=4.0 Hz), 130.61, 130.58, 129.90 (d, J=8.0 Hz), 129.40, 129.13 (d, J=2.0 Hz), 127.93, 127.37 (d, J=13.0 Hz), 125.75, 124.62 (d, J=4.0 Hz), 119.35, 116.24 (d, J=22 Hz), 51.79, 43.99, 21.09.

(E)-3-(2′-Fluoro-5-((4-methylbenzyl)carbamoyl)-[1,1′-biphenyl]-3-yl)acrylic acid (A1-e). White solid (75 mg, Yield 78%). ¹H NMR (400 MHZ, DMSO-d₆): δ 9.35 (1H, t, J=5.6 Hz, NH), 8.17 (1H, s, ArCH), 7.98 (1H, s, ArCH), 7.75 (1H, s, ArCH), 7.62 (1H, t, J=8.0 Hz, ArCH), 7.48-7.43 (1H, m, ArCH), 7.36-7.23 (5H, m, 4 ArCH, 1CH, Overlapped), 7.13 (2H, d, J=7.6 Hz, ArCH), 6.63 (1H, d, J=16.0 Hz, CH), 4.48 (2H, d, J=5.6 Hz, CH₂), 2.27 (3H, s, CH₃). ¹³C NMR (100 MHz, DMSO-d₆): δ 171.17, 166.02, 159.57 (d, J=245.0 Hz), 137.78, 137.08, 136.23, 136.06, 135.47, 135.24, 131.97, 131.40 (d, J=3.0 Hz), 130.64, 130.44 (d, J=9.0 Hz), 129.28, 128.02 (d, J=13.0 Hz), 127.81, 125.46 (d, J=4.0 Hz), 125.05, 116.58 (d, J=22.0 Hz), 42.96, 21.13. ¹⁹F NMR (376 MHZ, DMSO-d₆): −118.24. HRMS-ESI (m/z): [M+H]⁺ calcd for C₂₄H₂₁FNO₃, 390.1500; found, 390.1503.

Methyl (E)-3-(3′-fluoro-5-((4-methylbenzyl)carbamoyl)-[1,1′-biphenyl]-3-yl)acrylate (4f). White solid (120 mg, Yield 58%). ¹H NMR (400 MHZ, CDCl₃): δ 7.99 (1H, s, ArCH), 7.92 (1H, s, ArCH), 7.75 (1H, s, ArCH), 7.69 (1H, d, J=16.0 Hz, CH), 7.43-7.38 (1H, m, ArCH), 7.34 (2H, d, J=8.0 Hz, ArCH), 7.28-7.24 (3H, m, ArCH), 7.15 (2H, d, J=8.0 Hz, ArCH), 7.08 (1H, dt, J=8.0, 1.6 Hz, ArCH), 7.02 (1H, t, J=5.6 Hz, NH), 6.51 (1H, d, J=16.0 Hz, CH), 4.60 (2H, d, J=5.6 Hz, CH₂), 3.80 (3H, s, OCH₃), 2.33 (3H, s, CH₃). ¹³C NMR (100 MHZ, CDCl₃): δ 167.00, 166.59, 163.18 (d, J=245.0 Hz), 143.44, 141.39 (d, J=7.0 Hz), 141.06 (d, J=2.0 Hz), 137.39, 135.98, 135.42, 134.93, 130.53 (d, J=8.0 Hz), 129.45, 129.38, 127.99, 127.40, 125.68, 122.81 (d, J=3.0 Hz), 119.53, 114.98 (d, J=21.0 Hz), 114.10 (d, J=22.0), 51.84, 44.07, 21.10.

(E)-3-(3′-Fluoro-5-((4-methylbenzyl)carbamoyl)-[1,1′-biphenyl]-3-yl)acrylic acid (A1-f). White solid (55 mg, Yield 57%). ¹H NMR (400 MHZ, DMSO-d₆): δ 9.38 (1H, br s, NH), 8.15 (1H, s, ArCH), 8.12 (1H, s, ArCH), 7.96 (1H, s, ArCH), 7.69-7.64 (2H, m, ArCH), 7.56-7.50 (1H, m, ArCH), 7.33 (1H, d, J=16.0 Hz, CH), 7.27-7.23 (3H, m, ArCH), 7.14 (2H, d, J=7.6 Hz, ArCH), 6.69 (1H, d, J=16.0 Hz, CH), 4.49 (2H, d, J=5.2 Hz, CH₂), 2.27 (3H, s, CH₃). ¹³C NMR (100 MHZ, DMSO-d₆): δ 171.39, 166.09, 163.19 (d, J=242.0 Hz), 142.31 (d, J=8.0 Hz), 139.69, 138.17, 137.08, 136.23, 135.81, 135.58, 131.70, 131.34 (d, J=8.0 Hz), 129.29, 128.60, 127.81, 125.61, 123.48, 115.01 (d, J=20.0 Hz), 114.16 (d, J=22 Hz), 42.95, 21.14. ¹⁹F NMR (376 MHZ, DMSO-d₆): −112.71-−112.77 (m). HRMS-ESI (m/z): [M+H]⁺ calcd for C₂₄H₂₁FNO₃, 390.1500; found, 390.1503.

Methyl (E)-3-(2′,4′-difluoro-5-((4-methylbenzyl)carbamoyl)-[1,1′-biphenyl]-3-yl)acrylate (4g). White solid (160 mg, Yield 74%). ¹H NMR (400 MHZ, CDCl₃): δ 7.94 (1H, s, ArCH), 7.91 (1H, s, ArCH), 7.71 (1H, s, ArCH), 7.67 (1H, d, J=16.0 Hz, CH), 7.40-7.35 (1H, m, ArCH), 7.23 (2H, d, J=8.0 Hz, ArCH), 7.13 (2H, d, J=8.0 Hz, ArCH), 7.12 (1H, br s, NH), 6.96-6.89 (2H, m, ArCH), 6.49 (1H, d, J=16.0 Hz, CH), 4.57 (2H, d, J=5.2 Hz, CH₂), 3.78 (3H, s, OCH₃), 2.32 (3H, s, CH₃). ¹³C NMR (100 MHZ, CDCl₃): δ 167.01, 166.53, 162.73 (dd, J=250.0, 11.5 Hz), 159.70 (dd, J=250.0, 11.5 Hz), 143.42, 137.30, 136.07, 135.69, 135.08, 134.97, 131.40 (dd, J=9.0, 4.0 Hz), 131.29 (d, J=3.0 Hz), 129.41, 129.09, 127.92, 125.69, 123.69 (dd, J=13.0, 4.0 Hz), 119.49, 119.90 (t, J=25.5 Hz), 51.81, 44.01, 21.08.

(E)-3-(2′,4′-Difluoro-5-((4-methylbenzyl)carbamoyl)-[1,1′-biphenyl]-3-yl)acrylic acid (A1-g). White solid (72 mg, Yield 74%). ¹H NMR (400 MHZ, DMSO-d₆): δ 8.16 (1H, s, ArCH), 7.96 (1H, s, ArCH), 7.70 (1H, s, ArCH), 7.67-7.63 (1H, m, ArCH), 7.39-7.34 (1H, m, ArCH), 7.28 (1H, d, J=16.0 Hz, CH), 7.25-7.17 (3H, m, ArCH), 7.11 (2H, d, J=8.0 Hz, ArCH), 6.11 (1H, d, J=16.0 Hz, CH), 4.46 (2H, s, CH₂), 2.26 (3H, s, CH₃). ¹³C NMR (100 MHZ, DMSO-d₆): δ 171.30, 166.01, 162.34 (dd, J=245.5, 11.5 Hz), 159.62 (dd, J=247.5, 12.5 Hz), 137.65, 137.58, 136.28, 136.02, 135.43, 135.80, 132.55 (dd, J=10.5, 4.5 Hz), 131.73, 130.23, 129.20, 127.84, 125.25, 124.87, 124.80 (d, J=14.0 Hz), 122.71, 112.54 (d, J=24.0 Hz), 104.96 (t, J=26.0 Hz), 43.53, 21.12. ¹⁹F NMR (376 MHz, DMSO-d₆): −110.86, 113.83 (m). HRMS-ESI (m/z): [M+H]⁺ calcd for C₂₄H₂₀F₂NO₃, 408.1406; found, 408.1405.

Methyl (E)-3-(5-((4-methylbenzyl)carbamoyl)-4′-(trifluoromethyl)-[1,1′-biphenyl]-3-yl)acrylate (4h). White solid (165 mg, Yield 71%). ¹H NMR (500 MHZ, CDCl₃): δ 8.03 (1H, s, ArCH), 7.94 (1H, s, ArCH), 7.81 (1H, s, ArCH), 7.74 (1H, d, J=16.0 Hz, CH), 7.72-7.68 (4H, m, ArCH), 7.27 (2H, d, J=8.0 Hz, ArCH), 7.18 (2H, d, J=8.0 Hz, ArCH), 6.75 (1H, t, J=5.0 Hz, NH), 6.55 (1H, d, J=16.0 Hz, CH), 4.63 (2H, d, J=5.0 Hz, CH₂), 3.83 (3H, s, OCH₃), 2.36 (3H, s, CH₃). ¹³C NMR (125 MHZ, CDCl₃): δ 166.93, 166.45, 143.24, 142.93, 141.05, 137.58, 136.08, 135.63, 134.77, 129.58, 129.54, 128.04, 127.59, 127.52, 125.97, 125.95, 125.76, 119.82, 51.90, 44.17, 21.11.

(E)-3-(5-((4-Methylbenzyl)carbamoyl)-4′-(trifluoromethyl)-[1,1′-biphenyl]-3-yl)acrylic acid (A1-h). White solid (56 mg, Yield 58%). ¹H NMR (500 MHZ, DMSO-d₆): δ 9.21 (1H, t, J=6.0 Hz, NH), 8.26 (2H, s, ArCH), 8.24 (1H, s, ArCH), 8.05 (2H, d, J=16.0 Hz, ArCH), 7.86 (1H, d, J=8.0 Hz, ArCH), 7.74 (1H, d, J=16.0 Hz, CH), 7.25 (2H, d, J=8.0 Hz, ArCH), 7.15 (2H, d, J=8.0 Hz, ArCH), 6.78 (1H, d, J=16.0 Hz, CH), 4.50 (2H, d, J=6.0 Hz, CH₂), 2.28 (3H, s, CH₃). ¹³C NMR (125 MHZ, DMSO-d₆): δ 167.89, 165.73, 143.58, 139.83, 136.80, 136.38, 136.13, 135.90, 130.08, 129.34, 128.99, 128.87 (q, J=23.8 Hz), 128.30, 127.82, 127.76, 126.78, 126.30, 125.87, 123.70, 121.50, 43.02, 21.13, 21.14. ¹⁹F NMR (376 MHZ, DMSO-d₆): −60.93. HRMS-ESI (m/z): [M+H]⁺ calcd for C₂₅H₂₁F₃NO₃, 440.1468; found, 440.1469.

Methyl (E)-3-(4′-methyl-5-((4-methylbenzyl)carbamoyl)-[1,1′-biphenyl]-3-yl)acrylate (4i). White solid (150 mg, Yield 73%). ¹H NMR (500 MHZ, CDCl₃): δ 8.05 (1H, s, ArCH), 7.95 (1H, s, ArCH), 7.87 (1H, s, ArCH), 7.81 (1H, d, J=16.0 Hz, CH), 7.56 (2H, d, J=8.0 Hz, ArCH), 7.34 (4H, m, ArCH), 7.25 (2H, d, J=8.0 Hz, ArCH), 6.68 (1H, br s, NH), 6.59 (1H, d, J=16.0 Hz, CH), 4.70 (2H, d, J=5.5 Hz, CH₂), 3.89 (3H, s, OCH₃), 2.49 (3H, s, CH₃), 2.43 (3H, s, CH₃). ¹³C NMR (125 MHZ, CDCl₃): δ 167.10, 166.76, 143.79, 142.43, 138.11, 137.48, 136.56, 135.81, 135.31, 134.93, 129.73, 129.52, 129.36, 128.04, 127.17, 126.99, 124.84, 119.29, 51.83, 44.11, 21.14, 21.12.

(E)-3-(4′-Methyl-5-((4-methylbenzyl)carbamoyl)-[1,1′-biphenyl]-3-yl)acrylic acid (A1-i). White solid (65 mg, Yield 70%). ¹H NMR (500 MHZ, DMSO-d₆): δ 9.24 (1H, t, J=6.0 Hz, NH), 8.20 (1H, s, ArCH), 8.19 (1H, s, ArCH), 8.11 (1H, s, ArCH), 7.72 (1H, d, J=16.0 Hz, CH), 7.71 (2H, d, J=8.0 Hz, ArCH), 7.31 (2H, d, J=8.0 Hz, ArCH), 7.24 (2H, d, J=8.0 Hz, ArCH), 7.14 (2H, d, J=8.0 Hz, ArCH), 6.75 (1H, d, J=16.0 Hz, CH), 4.49 (2H, d, J=6.0 Hz, CH₂), 2.36 (3H, s, CH₃), 2.28 (3H, s, CH₃). ¹³C NMR (125 MHZ, DMSO-d₆): δ 168.00, 165.93, 143.56, 141.29, 137.94, 136.93, 136.46, 136.31, 135.91, 135.66, 130.05, 129.45, 129.32, 127.81, 127.25, 127.14, 125.52, 121.30, 42.97, 21.16, 21.14. HRMS-ESI (m/z): [M+H]⁺ calcd for C₂₅H₂₄NO₃, 386.1751; found, 386.1753.

Methyl (E)-3-(4′-ethyl-5-((4-methylbenzyl)carbamoyl)-[1,1′-biphenyl]-3-yl)acrylate (4j). White solid (130 mg, Yield 61%). ¹H NMR (500 MHZ, CDCl₃): δ 7.99 (1H, s, ArCH), 7.89 (1H, s, ArCH), 7.82 (1H, s, ArCH), 7.74 (1H, d, J=16.0 Hz, CH), 7.52 (2H, d, J=8.0 Hz, ArCH), 7.31-7.27 (4H, m, ArCH), 7.18 (2H, d, J=8.0 Hz, ArCH), 6.64 (1H, br s, NH), 6.54 (1H, d, J=16.0 Hz, CH), 4.64 (2H, d, J=5.0 Hz, CH₂), 3.83 (3H, s, OCH₃), 2.72 (2H, q, J=7.5 Hz, CH₂), 2.36 (3H, s, CH₃), 1.30 (3H, t, J=7.5 Hz, CH₃). ¹³C NMR (125 MHZ, CDCl₃): δ 167.10, 166.75, 144.47, 143.81, 142.46, 137.47, 136.81, 135.81, 135.30, 134.94, 129.51, 129.40, 128.54, 128.03, 127.20, 127.09, 124.86, 119.28, 51.83, 44.10, 28.54, 21.12, 15.54.

(E)-3-(4′-Ethyl-5-((4-methylbenzyl)carbamoyl)-[1,1′-biphenyl]-3-yl)acrylic acid (A1-j). White solid (56 mg, Yield 58%). ¹H NMR (500 MHZ, DMSO-d₆): δ 9.18 (1H, t, J=6.0 Hz, NH), 8.18 (1H, s, ArCH), 8.17 (1H, s, ArCH), 8.11 (1H, s, ArCH), 7.72 (1H, d, J=8.0 Hz, CH), 7.70 (1H, d, J=16.0 Hz, CH), 7.34 (2H, d, J=8.0 Hz), 7.25 (2H, d, J=8.0 Hz), 7.15 (2H, d, J=8.0 Hz), 6.74 (1H, d, J=16.0 Hz, CH), 4.49 (2H, d, J=6.0 Hz, CH₂), 2.66 (2H, q, J=7.5 Hz, CH₂), 2.27 (3H, s, CH₃), 1.22 (3H, t, J=7.5 Hz, CH₃). ¹³C NMR (125 MHZ, DMSO-d₆): δ 168.14, 165.95, 144.25, 143.40, 141.37, 136.91, 136.78, 136.33, 135.91, 135.71, 129.51, 129.33, 128.87, 127.80, 127.36, 127.18, 125.46, 121.51, 42.98, 28.30, 21.14, 16.04. HRMS-ESI (m/z): [M+H]⁺ calcd for C₂₆H₂₆NO₃, 400.1907; found, 400.1908.

Methyl (E)-3-(4′-isopropyl-5-((4-methylbenzyl)carbamoyl)-[1,1′-biphenyl]-3-yl)acrylate (4k). White solid (0.17 g, Yield 77%). ¹H NMR (500 MHz, CDCl₃): δ 7.98 (1H, s, ArCH), 7.89 (1H, s, ArCH), 7.83 (1H, s, ArCH), 7.77 (1H, d, J=16.0 Hz, CH), 7.54 (2H, d, J=8.5 Hz, ArCH), 7.35 (2H, d, J=8.0 Hz, ArCH), 7.29 (2H, d, J=7.5 Hz, ArCH), 7.20 (2H, d, J=7.5 Hz, ArCH), 6.57 (1H, d, J=16.0 Hz, CH), 6.46 (1H, br t, J=5.5 Hz, NH), 4.66 (2H, d, J=5.5 Hz, CH₂), 3.84 (3H, s, OCH₃), 3.00 (1H, m, CH), 2.38 (3H, s, CH₃), 1.31 (6H, d, J=7.0 Hz, (CH₃) 2). ¹³C NMR (125 MHZ, CDCl₃): δ 167.15, 166.94, 149.00, 143.90, 142.26, 137.24, 136.90, 135.79, 135.16, 135.10, 129.41, 129.35, 127.94, 127.38, 127.07, 124.97, 119.10, 51.78, 44.00, 33.83, 23.96, 21.11.

(E)-3-(4′-Isopropyl-5-((4-methylbenzyl)carbamoyl)-[1,1′-biphenyl]-3-yl)acrylic acid (A1-k). White solid (0.12 g, Yield 82%). ¹H NMR (500 MHZ, DMSO-d₆): δ 12.52 (1H, br s, COOH), 9.20 (1H, t, J=5.5 Hz, NH), 8.19 (2H, s, ArCH), 8.11 (1H, s, ArCH), 7.73 (1H, d, J=16.0 Hz, CH), 7.72 (2H, d, J=8.0 Hz, ArCH), 7.37 (2H, d, J=8.0 Hz, ArCH), 7.25 (2H, d, J=8.0 Hz, ArCH), 7.14 (2H, d, J=7.5 Hz, ArCH), 6.75 (1H, d, J=16.0 Hz, CH), 4.50 (2H, d, J=5.5 Hz, CH₂), 2.94 (1H, m, CH), 2.28 (3H, s, CH₃), 1.24 (6H, d, J=7.0 Hz, (CH₃) 2). ¹³C NMR (125 MHZ, DMSO-d₆): δ 167.94, 165.93, 148.83, 143.76, 141.43, 136.96, 136.91, 136.32, 135.92, 135.60, 129.62, 129.32, 127.81, 127.41, 127.39, 127.34, 125.48, 121.07, 42.99, 33.61, 24.28, 28.14. HRMS-ESI (m/z): [M+H]⁺ calcd for C₂₇H₂₈NO₃, 414.2064; found, 414.2063.

Methyl (E)-3-(4′-methoxy-5-((4-methylbenzyl)carbamoyl)-[1,1′-biphenyl]-3-yl)acrylate (4l). White solid (130 mg, Yield 61%). ¹H NMR (400 MHZ, CDCl₃): δ 7.97 (1H, s, ArCH), 7.85 (1H, s, ArCH), 7.75 (1H, s, ArCH), 7.71 (1H, d, J=16.0 Hz, CH), 7.51 (2H, d, J=8.8 Hz, ArCH), 7.27 (2H, d, J=8.0 Hz, ArCH), 7.17 (2H, d, J=8.0 Hz, ArCH), 6.99 (2H, d, J=8.8 Hz, ArCH), 6.83 (1H, J=5.6 Hz, NH), 6.52 (1H, d, J=16.0 Hz, CH), 4.61 (2H, d, J=5.2 Hz, CH₂), 3.86 (3H, s, OCH₃), 3.81 (3H, s, OCH₃), 2.34 (3H, s, CH₃). ¹³C NMR (100 MHZ, CDCl₃): δ 167.13, 166.85, 159.78, 143.85, 142.00, 137.39, 135.79, 135.22, 135.00, 131.86, 129.47, 129.07, 128.23, 128.00, 127.00, 124.49, 119.16, 114.42, 55.37, 51.81, 44.05, 21.12.

(E)-3-(4′-Methoxy-5-((4-methylbenzyl)carbamoyl)-[1,1′-biphenyl]-3-yl)acrylic acid (A1-l). White solid (55 mg, Yield 57%). ¹H NMR (400 MHZ, DMSO-d₆): δ 9.28 (1H, br s, NH), 8.19-8.11 (3H, m, ArCH), 7.78 (3H, br s, 2ArCH, 1CH), 7.27-7.09 (6H, m, ArCH), 6.78 (1H, br s, CH), 4.51 (2H, s, CH₂), 3.84 (3H, s, OCH₃), 2.30 (3H, s, CH₃). ¹³C NMR (100 MHZ, DMSO-d₆): δ 168.02, 165.98, 159.84, 143.67, 141.04, 136.97, 136.31, 135.90, 135.62, 131.68, 129.32, 129.23, 128.61, 127.83, 126.92, 125.12, 121.21, 114.89, 55.72, 42.97, 21.15. HRMS-ESI (m/z): [M+H]⁺ calcd for C₂₅H₂₄NO₄, 402.1700; found, 402.1705.

Methyl (E)-3-(4′-cyano-5-((4-methylbenzyl)carbamoyl)-[1,1′-biphenyl]-3-yl)acrylate (4m). White solid (70 mg, Yield 70%). ¹H NMR (500 MHZ, CDCl₃): δ 8.03 (1H, s, ArCH), 7.95 (1H, s, ArCH), 7.82 (1H, s, ArCH), 7.77-7.72 (4H, m, ArCH), 7.71 (1H, d, J=16.0 Hz, CH), 7.28 (2H, d, J=8.0 Hz, ArCH), 7.19 (2H, d, J=8.0 Hz, ArCH), 6.63 (1H, t, J=5.5 Hz, NH), 6.57 (1H, d, J=16.0 Hz, CH), 4.64 (2H, d, J=5.5 Hz, CH₂), 3.83 (3H, s, OCH₃), 2.36 (3H, s, CH₃). ¹³C NMR (125 MHZ, CDCl₃): δ 166.84, 166.25, 143.90, 143.04, 140.47, 137.57, 136.21, 135.76, 134.76, 132.79, 129.52, 129.48, 128.04, 127.95, 127.85, 127.60, 126.13, 120.02, 118.56, 111.89, 51.94, 44.16, 21.12.

(E)-3-(4′-Cyano-5-((4-methylbenzyl)carbamoyl)-[1,1′-biphenyl]-3-yl)acrylic acid (A1-m). White solid (45 mg, Yield 67%). ¹H NMR (500 MHZ, CDCl₃+CD₃OD): δ 8.48 (1H, t, J=6.0 Hz, NH), 7.99 (1H, s, ArCH), 7.95 (1H, s, ArCH), 7.52-7.46 (5H, m, ArCH), 7.41 (1H, d, J=16.0 Hz, CH), 6.97 (2H, d, J=8.0 Hz, ArCH), 6.84 (2H, d, J=8.0 Hz, ArCH), 6.34 (1H, d, J=16.0 Hz, CH), 4.29 (2H, d, J=6.0 Hz, CH₂), 2.03 (3H, s, CH₃). ¹³C NMR (125 MHZ, CDCl₃+CD₃OD): δ 172.90, 170.83, 148.98, 144.46, 141.41, 140.77, 140.66, 140.42, 137.47, 137.40, 137.35, 134.34, 134.21, 133.88, 132.72, 132.66, 132.61, 132.53, 125.77, 123.43, 116.12, 48.24, 25.82. HRMS-ESI (m/z): [M+H]⁺ calcd for C₂₅H₂₁N₂O₃, 397.1547; found, 397.1549.

Methyl (E)-3-(5-((4-methylbenzyl)carbamoyl)-4′-phenoxy-[1,1′-biphenyl]-3-yl)acrylate (4n). White solid (0.35 g, Yield 71%). ¹H NMR (500 MHZ, CDCl₃): δ 7.98 (1H, s, ArCH), 7.87 (1H, s, ArCH), 7.81 (1H, s, ArCH), 7.77 (1H, d, J=16.0 Hz, CH), 7.57 (2H, d, J=8.5 Hz, ArCH), 7.39 (2H, t, J=8.0 Hz, ArCH), 7.29 (2H, d, J=7.5 Hz, ArCH), 7.21-7.16 (3H, m, ArCH), 7.12-7.06 (4H, m, ArCH), 6.56 (1H, d, J=16.0 Hz, CH), 6.49 (1H, t, J=5.5 Hz, NH), 4.65 (2H, d, J=5.5 Hz, CH₂), 3.84 (3H, s, OCH₃), 2.38 (3H, s, CH₃). ¹³C NMR (125 MHZ, CDCl₃): δ 167.04, 166.63, 157.70, 156.80, 143.66, 141.91, 137.57, 135.89, 135.42, 134.86, 134.36, 129.88, 129.55, 129.29, 128.56, 128.07, 127.11, 124.79, 123.68, 119.45, 119.21, 119.08, 51.86, 44.15, 21.13.

(E)-3-(5-((4-Methylbenzyl)carbamoyl)-4′-phenoxy-[1,1′-biphenyl]-3-yl)acrylic acid (A1-n). White solid (0.17 g, Yield 58%). ¹H NMR (500 MHZ, CDCl₃+CD₃OD): δ 8.07 (1H, s, ArCH), 7.99 (1H, s, ArCH), 7.83 (1H, s, ArCH), 7.73 (1H, d, J=16.0 Hz, CH), 7.59 (2H, d, J=8.0 Hz, ArCH), 7.34 (2H, t, J=8.0 Hz, ArCH), 7.23 (2H, d, J=7.5 Hz, ArCH), 7.12-7.11 (3H, m, ArCH), 7.06-7.01 (4H, m, ArCH), 6.56 (1H, d, J=16.0 Hz, CH), 4.55 (2H, s, CH₂), 2.29 (3H, s, CH₃). ¹³C NMR (125 MHZ, CDCl₃+CD₃OD): δ 172.78, 171.76, 161.56, 160.79, 148.04, 145.54, 140.71, 139.51, 139.30, 138.40, 133.64, 133.13, 132.98, 132.35, 131.44, 131.30, 128.89, 127.45, 123.64, 122.93, 122.77, 47.36, 24.41. HRMS-ESI (m/z): [M+H]⁺ calcd for C₃₀H₂₆NO₄, 464.1856; found, 464.1859.

Methyl (E)-3-(3-((4-methylbenzyl)carbamoyl)-5-(naphthalen-2-yl)phenyl)acrylate (4o). White solid (160 mg, Yield 71%). ¹H NMR (400 MHZ, CDCl₃): δ 8.14 (1H, s, ArCH), 8.04 (1H, s, ArCH), 7.94-7.87 (5H, m, ArCH), 7.77 (1H, d, J=16.0 Hz, CH), 7.71 (1H, dd, J=8.4, 1.6 Hz, ArCH), 7.56-7.51 (2H, m, ArCH), 7.30 (2H, d, J=7.6 Hz, ArCH), 7.19 (2H, d, J=7.6 Hz, ArCH), 6.75 (1H, t, J=5.2 Hz, NH), 6.76 (1H, d, J=16.0 Hz, CH), 4.65 (2H, d, J=5.6 Hz, CH₂), 3.83 (3H, s, OCH₃), 2.36 (3H, s, CH₃). ¹³C NMR (100 MHZ, CDCl₃): δ 167.07, 166.72, 143.70, 142.36, 137.49, 136.68, 135.93, 135.42, 134.95, 133.54, 132.93, 129.74, 129.52, 128.80, 128.28, 128.06, 127.70, 127.64, 126.62, 126.47, 126.19, 125.13, 125.04, 119.42, 51.86, 44.13, 21.13.

(E)-3-(3-((4-Methylbenzyl)carbamoyl)-5-(naphthalen-2-yl)phenyl)acrylic acid (A1-o). White solid (62 mg, Yield 64%). ¹H NMR (400 MHZ, DMSO-d₆): δ 12.51 (1H, br s, OH), 9.26 (1H, t, J=6.0 Hz, NH), 8.39 (2H, d, J=7.6 Hz, ArCH), 8.32 (1H, s, ArCH), 8.24 (1H, s, ArCH), 8.07-7.99 (4H, m, ArCH), 7.77 (1H, d, J=16.0 Hz, CH), 7.60-7.54 (2H, m, ArCH), 7.27 (2H, d, J=8.0 Hz, ArCH), 7.16 (2H, d, J=8.0 Hz, ArCH), 6.80 (1H, d, J=16.0 Hz, CH), 4.52 (2H, d, J=5.6 Hz, CH₂), 2.29 (3H, s, CH₃). ¹³C NMR (100 MHZ, DMSO-d₆): δ 167.95, 165.93, 143.68, 141.22, 136.91, 136.66, 136.35, 136.06, 135.77, 133.73, 132.98, 129.98, 129.34, 129.03, 128.74, 128.01, 127.84, 127.71, 127.02, 126.90, 126.27, 126.00, 125.55, 121.25, 43.02, 21.15. HRMS-ESI (m/z): [M+H]⁺ calcd for C₂₈H₂₄NO₃, 422.1751; found, 422.1752.

Methyl (E)-3-(3-((4-methylbenzyl)carbamoyl)-5-(quinolin-7-yl)phenyl)acrylate (4p). White solid (0.11 g, Yield 69%). ¹H NMR (500 MHZ, CDCl₃): δ 8.83 (1H, s, ArCH), 8.25-8.13 (3H, m, ArCH), 7.96 (1H, s, ArCH), 7.88 (2H, br s, ArCH), 7.77 (1H, s, ArCH), 7.69 (1H, d, J=16.0 Hz, CH), 7.43 (1H, s, ArCH), 7.29 (2H, br s, ArCH), 7.16-7.12 (3H, m, ArCH), 6.50 (1H, d, J=16.0 Hz, CH), 4.65 (2H, s, CH₂), 3.81 (3H, s, OCH₃), 2.33 (3H, s, CH₃). ¹³C NMR (125 MHZ, CDCl₃): δ 166.98, 166.59, 150.74, 147.86, 143.41, 141.27, 140.63, 137.40, 136.26, 136.16, 135.56, 134.97, 129.66, 129.49, 128.62, 128.09, 127.76, 127.60, 127.00, 125.95, 121.43, 119.58, 51.85, 44.14, 21.11.

(E)-3-(3-((4-Methylbenzyl)carbamoyl)-5-(quinolin-7-yl)phenyl)acrylic acid (A1-p). White solid (70 mg, Yield 73%). ¹H NMR (500 MHZ, DMSO-d₆): δ 12.54 (1H, br s, OH), 9.35 (1H, br s, NH), 8.98 (1H, s, ArCH), 8.50 (1H, s, ArCH), 8.46-8.38 (3H, m, ArCH), 8.28 (1H, s, ArCH), 8.13 (2H, s, ArCH), 7.78 (1H, d, J=16.0 Hz, CH), 7.58 (1H, dd, J=4.0, 8.0 Hz, ArCH), 7.27 (2H, d, J=7.5 Hz, ArCH), 7.15 (2H, d, J=7.5 Hz, ArCH), 6.83 (1H, d, J=16.0 Hz, CH), 4.51 (2H, d, J=5.5 Hz, CH₂), 2.28 (3H, s, CH₃). ¹³C NMR (125 MHZ, DMSO-d₆): δ 167.94, 165.80, 151.53, 148.21, 143.57, 140.54, 140.41, 136.91, 136.56, 136.33, 136.11, 135.94, 130.19, 129.34, 127.93, 127.83, 126.88, 126.64, 126.33, 122.21, 121.42, 43.01, 21.15. HRMS-ESI (m/z): [M+H]⁺ calcd for C₂₇H₂₃N₂O₃, 423.1703; found, 423.1707.

Methyl (E)-3-(3-((4-methylbenzyl)carbamoyl)-5-(quinoxalin-6-yl)phenyl)acrylate (4q). White solid (0.09 g, Yield 82%). ¹H NMR (500 MHZ, CDCl₃): δ 8.85 (2H, d, J=7.5 Hz, ArCH), 8.25 (1H, s, ArCH), 8.15 (2H, d, J=4.5 Hz, ArCH), 7.98 (2H, d, J=4.5 Hz, ArCH), 7.92 (1H, s, ArCH), 7.72 (1H, d, J=16.0 Hz, CH), 7.27 (2H, d, J=8.0 Hz, ArCH), 7.16 (2H, d, J=8.0 Hz, ArCH), 6.97 (1H, br s, NH), 6.54 (1H, d, J=16.0 Hz, CH), 4.64 (2H, d, J=5.5 Hz, CH₂), 3.81 (3H, s, OCH₃), 2.33 (3H, s, CH₃). ¹³C NMR (125 MHZ, CDCl₃): δ 166.92, 166.43, 145.67, 145.22, 143.25, 143.07, 142.57, 141.02, 140.76, 137.48, 136.24, 135.71, 134.84, 130.20, 129.74, 129.51, 129.31, 128.07, 127.71, 127.36, 126.06, 119.82, 51.89, 44.18, 21.11.

(E)-3-(3-((4-Methylbenzyl)carbamoyl)-5-(quinoxalin-6-yl)phenyl)acrylic acid (A1-q). White solid (65 mg, Yield 75%). ¹H NMR (500 MHZ, DMSO-d₆): δ 9.31 (1H, t, J=6.0 Hz, NH), 9.02 (1H, d, J=1.5 Hz, ArCH), 8.98 (1H, d, J=1.5 Hz, ArCH), 8.58 (1H, d, J=1.5 Hz, ArCH), 8.43 (2H, d, J=5.5 Hz, ArCH), 8.37 (1H, dd, J=9.0, 1.5 Hz, ArCH), 8.28 (1H, s, ArCH), 8.23 (1H, d, J=7.5 Hz, ArCH), 7.78 (1H, d, J=16.0 Hz, CH), 7.27 (2H, d, J=8.0 Hz, ArCH), 7.16 (2H, d, J=8.0 Hz, ArCH), 6.84 (1H, d, J=16.0 Hz, CH), 4.52 (2H, d, J=6.0 Hz, CH₂), 2.28 (3H, s, CH₃). ¹³C NMR (125 MHZ, DMSO-d₆): δ 167.93, 165.74, 146.83, 146.38, 143.47, 142.99, 142.36, 140.90, 139.84, 136.86, 136.36, 136.16, 136.01, 130.29, 130.21, 129.93, 129.35, 127.90, 127.83, 127.26, 126.99, 121.53, 43.02, 21.15. HRMS-ESI (m/z): [M+H]⁺ calcd for C₂₆H₂₂N₃O₃, 424.1656; found, 424.1659.

Methyl (E)-3-(3-([1,2,4]triazolo[1,5-a]pyridin-6-yl)-5-((4-methylbenzyl)carbamoyl)phenyl)acrylate (4r). White solid (110 mg, Yield 50%). ¹H NMR (400 MHz, CDCl₃): δ 8.84 (1H, s, ArCH), 8.39 (1H, s, ArCH), 8.08 (1H, s, ArCH), 7.99 (1H, s, ArCH), 7.81 (3H, br s, ArCH), 7.73 (1H, d, J=16.0 Hz, CH), 7.28 (2H, d, J=7.6 Hz, ArCH), 7.17 (2H, d, J=7.6 Hz, ArCH), 6.96 (1H, br s, NH), 6.56 (1H, d, J=16.0 Hz, CH), 4.64 (2H, d, J=0.4 Hz, CH₂), 3.83 (3H, s, OCH₃), 2.35 (3H, s, CH₃). ¹³C NMR (125 MHz, CDCl₃): δ 166.78, 166.07, 154.02, 142.84, 137.58, 137.30, 136.52, 136.03, 134.77, 130.15, 129.53, 129.25, 128.08, 127.53, 127.45, 126.32, 126.12, 120.27, 116.75, 51.98, 44.19, 21.13.

(E)-3-(3-([1,2,4]triazolo[1,5-a]pyridin-6-yl)-5-((4-methylbenzyl)carbamoyl)phenyl)acrylic acid (A1-r). White solid (60 mg, yield 75%). ¹H NMR (500 MHZ, DMSO-d₆): δ 9.52 (1H, s, ArCH), 9.19 (1H, t, J=6.0 Hz, NH), 8.58 (1H, s, ArCH), 8.36 (1H, s, ArCH), 8.32 (1H, s, ArCH), 8.21-8.19 (2H, m, ArCH), 7.99 (1H, d, J=9.5 Hz), 7.69 (1H, d, J=16.0 Hz, CH), 7.26 (2H, d, J=8.0 Hz, ArCH), 7.26 (2H, d, J=8.0 Hz, ArCH), 6.83 (1H, d, J=16.0 Hz, CH), 4.51 (2H, s, CH₂), 2.23 (3H, s, CH₃). ¹³C NMR (125 MHZ, DMSO-d₆): δ 167.93, 165.73, 155.01, 143.34, 136.80, 136.40, 136.20, 135.98, 130.48, 129.51, 129.37, 127.82, 127.45, 127.38, 127.15, 126.78, 121.63, 116.62, 43.02, 21.14. HRMS-ESI (m/z): [M+H]⁺ calcd for C₂₄H₂₁N₄O₃, 413.1608; found, 413.1617.

Methyl (E)-3-(3-(2,3-dihydrobenzo[b][1,4]dioxin-6-yl)-5-((4-methylbenzyl)carbamoyl)phenyl)acrylate (4s). White solid (0.17 g, Yield 77%). ¹H NMR (500 MHz, CDCl₃+CD₃OD): δ 7.90 (1H, s, ArCH), 7.83 (1H, s, ArCH), 7.70 (1H, s, ArCH), 7.68 (1H, d, J=16.0 Hz, CH), 7.22 (2H, d, J=8.0 Hz, ArCH), 7.12 (2H, d, J=8.0 Hz, ArCH), 7.07 (1H, d, J=1.5 Hz, ArCH), 7.04 (1H, dd, J=8.5, 1.6 Hz, ArCH), 6.89 (1H, d, J=8.0 Hz, ArCH), 6.48 (1H, d, J=16.0 Hz, CH), 4.56 (2H, s, CH₂), 4.25 (4H, s, (OCH₂) 2), 3.77 (3H, s, OCH₃), 2.30 (3H, s, CH₃). ¹³C NMR (125 MHZ, CDCl₃+CD₃OD) δ 167.31, 166.93, 143.94, 143.86, 143.82, 141.77, 137.35, 135.65, 135.19, 134.94, 132.90, 129.42, 129.11, 127.95, 127.02, 124.79, 120.16, 119.11, 117.77, 115.89, 64.46, 64.39, 51.83, 43.89, 21.05.

(E)-3-(3-(2,3-Dihydrobenzo[b][1,4]dioxin-6-yl)-5-((4-methylbenzyl)carbamoyl)phenyl)acrylic acid (A1-s). White solid (85 mg, Yield 77%). ¹H NMR (500 MHZ, DMSO-d₆): δ 12.49 (1H, br s, OH), 9.18 (1H, t, J=5.5 Hz), NH), 8.14 (2H, s, ArCH), 8.08 (1H, s, ArCH), 7.71 (1H, d, J=16.0 Hz, CH), 7.35 (1H, d, J=1.5 Hz, ArCH), 7.29 (1H, dd, J=1.5, 8.0 Hz, ArCH), 7.24 (2H, d, J=8.0 Hz, ArCH), 7.14 (2H, d, J=8.0 Hz, ArCH), 6.97 (1H, d, J=8.0 Hz, ArCH), 6.74 (1H, d, J=16.0 Hz, CH), 4.49 (2H, d, J=5.5 Hz, CH₂), 4.29 (4H, s, (OCH₂) 2), 2.28 (3H, s, CH₃). ¹³C NMR (125 MHZ, DMSO-d₆): δ 167.96, 165.91, 144.23, 144.09, 143.77, 140.81, 136.91, 136.33, 135.83, 135.58, 132.57, 129.33, 129.25, 127.78, 126.88, 125.42, 121.04, 120.33, 117.99, 115.98, 64.68, 64.60, 42.97, 21.14. HRMS-ESI (m/z): [M+H]⁺ calcd for C₂₆H₂₄NO₅, 430.1649; found, 430.1648.

(Methyl (E)-3-(3-(benzo[d][1,3]dioxol-5-yl)-5-((4-methylbenzyl)carbamoyl)phenyl)acrylate (4t). White solid (130 mg, Yield 59%). ¹H NMR (400 MHz, CDCl₃): δ 7.92 (1H, d, J=1.6 Hz, ArCH), 7.86 (1H, s, ArCH), 7.72 (1H, d, J=1.6 Hz, ArCH), 7.71 (1H, d, J=16.0 Hz, CH), 7.27 (2H, d, J=8.0 Hz, ArCH), 7.17 (2H, d, J=8.0 Hz, ArCH), 7.06-7.04 (2H, m, ArCH), 6.88 (1H, d, J=8.0 Hz, ArCH), 6.73 (1H, t, J=5.6 Hz, NH), 6.52 (1H, d, J=16.0 Hz, CH), 6.01 (2H, s, CH₂), 4.62 (2H, d, J=5.6 Hz, CH₂), 3.82 (3H, s, OCH₃), 2.34 (3H, s, CH₃). ¹³C NMR (100 MHZ, CDCl₃): δ 167.08, 166.71, 148.36, 147.78, 143.70, 142.12, 137.45, 135.82, 135.28, 134.94, 133.71, 129.49, 129.22, 128.02, 127.10, 124.76, 120.88, 119.30, 108.73, 107.56, 101.38, 51.83, 44.08, 21.12.

(E)-3-(3-(Benzo[d][1,3]dioxol-5-yl)-5-((4-methylbenzyl)carbamoyl)phenyl)acrylic acid (A1-t). White solid (62 mg, Yield 64%). ¹H NMR (400 MHZ, DMSO-d₆): δ 9.16 (1H, t, J=5.6 Hz, NH), 8.12 (2H, s, ArCH), 8.08 (1H, s, ArCH), 7.67 (1H, d, J=16.0 Hz, CH), 7.43 (1H, s, ArCH), 7.31 (1H, d, J=8.0 Hz, ArCH), 7.24 (2H, d, J=7.6 Hz, ArCH), 7.15 (2H, d, J=8.0 Hz, ArCH), 7.04 (1H, d, J=8.0 Hz, ArCH), 6.75 (1H, d, J=16.0 Hz, CH), 6.09 (2H, s, CH₂), 4.49 (2H, d, J=5.6 Hz, CH₂), 2.28 (3H, s, CH₃). ¹³C NMR (100 MHZ, DMSO-d₆): δ 168.14, 165.94, 148.54, 147.79, 143.10, 141.04, 136.90, 136.33, 135.84, 135.76, 133.59, 129.33, 127.79, 126.91, 125.47, 121.91, 121.19, 109.15, 107.85, 101.75, 42.97, 21.14. HRMS-ESI (m/z): [M+H]⁺ calcd for C₂₅H₂₂NO₅, 416.1492; found, 416.1492.

Methyl (E)-3-(5-((4-methylbenzyl)carbamoyl)-[1,1′:4′,1″-terphenyl]-3-yl)acrylate (4u): A transparent oil (74%). ¹H NMR (400 MHZ; CDCl₃): δ 7.88 (1H, s, CH), 7.74 (1H, s, CH), 7.38-7.50 (6H, m, ArCH and CH), 7.14-7.21 (8H, m, ArCH), 6.26 (1H, d, J=16.0 Hz, CH), 4.49 (2H, s, CH₂), 3.89 (3H, s, CH₃), 2.32 (3H, s, CH₃). ¹³C NMR (100 MHz; CDCl₃): δ 141.09, 141.07, 139.61, 139.28, 137.29, 135.00, 133.98, 133.32, 132.95, 130.64, 128.71, 128.39, 128.11, 127.22, 126.22, 126.00, 125.62, 124.90, 123.04, 118.00, 41.41, 18.23.

(E)-3-(5-((4-Methylbenzyl)carbamoyl)-[1,1′:4′,1″-terphenyl]-3-yl)acrylic acid (A1-u): A white solid (71%). ¹H NMR (400 MHZ; CD₃OD): δ 7.88 (1H, s, CH), 7.74 (1H, s, CH), 7.38-7.50 (6H, m, ArCH and CH), 7.14-7.21 (8H, m, ArCH), 6.26 (1H, d, J=16.00 Hz, CH), 4.49 (2H, s, CH₂). ¹³C NMR (100 MHz; CD₃OD): δ 167.00, 165.00, 141.09, 141.07, 139.61, 139.28, 137.29, 135.00, 133.98, 133.32, 132.95, 130.64, 128.71, 128.39, 128.11, 127.22, 126.22, 126.00, 125.62, 124.90, 123.04, 118.00, 41.41, 18.23. HRMS-ESI (m/z): [M+H]⁺ calcd for C₃₀H₂₆NO₃, 448.1907; found, 448.1918.

Methyl (E)-3-(4′-(9H-carbazol-9-yl)-5-((4-methylbenzyl)carbamoyl)-[1,1′-biphenyl]-3-yl)acrylate (4v): A transparent oil (74%). ¹H NMR (400 MHZ; CDCl₃): δ 8.17 (1H, s, ArCH). 7.88 (1H, s, ArCH), 7.74 (1H, s, CH), 7.38-7.62 (10H, m, ArCH), 7.14-7.21 (7H, m, ArCH), 6.26 (1H, d, J=16.0 Hz, CH), 4.49 (2H, s, CH₂), 3.89 (3H, s, CH₃), 2.32 (3H, s, CH₃). ¹³C NMR (100 MHz; CDCl₃): δ 141.09, 141.07, 139.61, 139.28, 137.29, 135.00, 133.98, 133.32, 132.95, 130.64, 128.71, 128.39, 128.11, 127.22, 126.22, 126.00, 125.62, 124.90, 123.04, 118.00, 41.41, 18.23.

(E)-3-(4′-(9H-Carbazol-9-yl)-5-((4-methylbenzyl)carbamoyl)-[1,1′-biphenyl]-3-yl)acrylic acid (A1-v): A white solid (71%). ¹H NMR (400 MHZ; CD₃OD): δ 2.32 (3H, s, CH₃), 4.49 (2H, s, CH₂), 6.26 (1H, d, J=16.0 Hz, CH), 7.14-7.21 (7H, m, ArCH), 7.38-7.62 (10H, m, ArCH), 7.74 (1H, s, CH), 7.88 (1H, s, ArCH), 8.17 (1H, s, ArCH). ¹³C NMR (100 MHZ; CD₃OD): δ 18.23, 41.41, 118.00, 123.04, 124.90, 125.62, 126.00, 126.22, 127.22, 128.11, 128.39, 128.71, 130.64, 132.95, 133.32, 133.98, 135.00, 137.29, 139.28, 139.61, 141.07, 141.09, 166.71, 167.07. HRMS-ESI (m/z): [M+H]⁺ calcd for C₃₆H₂₉N₂O₃, 537.2173; found, 537.2188.

Methyl (E)-3-(3-((4-methylbenzyl)carbamoyl)-5-(phenylethynyl)phenyl)acrylate (4w): A transparent oil (72%). ¹H NMR (400 MHZ; CDCl₃): δ 2.32 (3H, s, CH₃), 3.89 (3H, s, CH₃), 4.64 (2H, d, J=7.4 Hz, CH₂), 6.54 (1H, s, CH), 7.15 (2H, d, J=7.8 Hz, ArCH), 7.24 (2H, d, J=8.0 Hz, ArCH), 7.28-7.39 (5H, m, ArCH), 7.53-7.54 (4H, m, CH), 7.63 (1H, s, ArCH), 7.83 (1H, s, ArCH), 7.98 (1H, s, ArCH). 8.01 (1H, s, ArCH). ¹³C NMR (100 MHZ; CDCl₃): δ 63.7, 69.9, 114.4, 118.6, 119.3, 122.1, 122.1, 124.6, 128.9, 129.2, 133.6, 137.8, 142.9, 159.5, 166.5.

(E)-3-(3-((4-methylbenzyl)carbamoyl)-5-(phenylethynyl)phenyl)acrylic acid (A1-w): A yellow solid (72%). ¹H NMR (400 MHZ; CD₃OD): δ 2.32 (3H, s, CH₃), 4.64 (2H, d, J=7.4 Hz, CH₂), 6.54 (1H, s, CH), 7.15 (2H, d, J=7.8 Hz, ArCH), 7.24 (2H, d, J=8.0 Hz, ArCH), 7.28-7.39 (5H, m, ArCH), 7.53-7.54 (4H, m, ArCH), 7.63 (1H, s, CH), 7.83 (1H, s, ArCH), 7.98 (1H, s, ArCH), 8.01 (1H, s, ArCH), ¹³C NMR (100 MHz; CD₃OD): δ 63.7, 69.9, 114.4, 118.6, 119.3, 122.1, 122.1, 124.6, 128.9, 129.2, 133.6, 137.8, 142.9, 159.5, 166.5. HRMS-ESI (m/z): [M+H]⁺ calcd for C₂₆H₂₂NO₃, 396.1600; found, 396.1603.

Methyl (E)-3-(3-((4-methylbenzyl)carbamoyl)-5-(2-phenylpropyl)phenyl)acrylate (4x): A transparent oil (82%). ¹H NMR (400 MHZ; CDCl₃): δ 1.20 (3H, s, CH₃), 1.28-1.32 (2H, m, J=7.4 Hz, CH₂), 1.40 (1H, d, J=7.4 Hz, CH), 2.39 (3H, s, CH₃), 3.89 (3H, s, CH₃), 4.64 (2H, d, J=7.4 Hz, CH₂), 6.54 (1H, s, CH), 7.19-7.28 (7H, m, ArCH), 7.50-7.76 (7H, m, ArCH and CH), 7.02 (1H, s, ArCH), 7.08 (1H, s, ArCH), 7.89 (1H, s, ArCH). ¹³C NMR (100 MHz; CDCl₃): δ 29.2, 33.4, 36.0, 61.9, 87.8, 127.3, 128.9, 132.5, 135.6, 136.1, 136.3, 138.9, 139.2, 145.6, 148.7, 150.1, 150.5, 151.5, 152.1, 153.1, 163.3, 180.2.

(E)-3-(3-((4-Methylbenzyl)carbamoyl)-5-(2-phenylpropyl)phenyl)acrylic acid (A1-x): A yellow solid (57%). ¹H NMR (400 MHZ; CD₃OD): δ 1.18 (3H, s, CH₃), 1.26-1.30 (2H, m, J=7.4 Hz, CH₂), 1.38 (1H, d, J=7.4 Hz, CH), 2.37 (3H, s, CH₃), 3.87 (3H, s, CH₃), 4.62 (2H, d, J=7.4 Hz, CH₂), 6.52 (1H, s, CH), 7.17-7.26 (7H, m, ArCH), 7.48-7.74 (7H, m, ArCH and CH), 7.02 (1H, s, ArCH), 7.08 (1H, s, ArCH), 7.89 (1H, s, ArCH). ¹³C NMR (100 MHz; CD₃OD): δ 29.2, 33.4, 36.0, 61.9, 87.8, 127.3, 128.9, 132.5, 135.6, 136.1, 136.3, 138.9, 139.2, 145.6, 148.7, 150.1, 150.5, 151.5, 152.1, 153.1, 163.3, 180.2. HRMS-ESI (m/z): [M+H]⁺ calcd for C₂₇H₂₈NO₃, 414.2069; found, 414.2073.

Methyl (E)-3-(3′-fluoro-5-((4-methylbenzyl)carbamoyl)-4′-(morpholinomethyl)-[1,1′-biphenyl]-3-yl)acrylate (4y): A yellow solid (78%). ¹H NMR (400 MHZ; CDCl₃): δ 2.32 (3H, s, CH₃), 2.64 (2H, t, J=7.4 Hz, CH₂), 3.68-3.87 (8H, m, CH₂), 3.89 (3H, s, CH₃), 4.58 (2H, t, J=7.4 Hz, CH₂), 6.65 (1H, d, J=15.9 Hz, CH), 7.15-7.28 (4H, m, ArCH), 7.50-7.76 (5H, m, ArCH and CH), 8.02 (1H, s, ArCH), 8.08 (1H, s, ArCH), 8.89 (1H, s, CH). ¹³C NMR (100 MHz; CDCl₃): 0 28.2, 31.4, 39.0, 64.9, 80.8, 120.3, 120.9, 122.5, 125.6, 126.1, 126.3, 128.9, 129.2, 135.6, 138.7, 140.1, 140.5, 141.5, 142.1, 143.1, 166.3, 181.2.

(E)-3-(3′-Fluoro-5-((4-methylbenzyl)carbamoyl)-4′-(morpholinomethyl)-[1,1′-biphenyl]-3-yl)acrylic acid (A1-y): A yellow solid (60%). ¹H NMR (400 MHZ; CD₃OD): δ 2.32 (3H, s, CH₃), 2.64 (2H, t, J=7.4 Hz, CH₂), 3.68-3.87 (8H, m, CH₂), 4.58 (2H, t, J=7.4 Hz, CH₂), 6.65 (1H, d, J=15.9 Hz, CH), 7.15-7.28 (4H, m, ArCH), 7.50-7.76 (5H, m, ArCH and CH), 8.02 (1H, s, ArCH), 8.08 (1H, s, ArCH), 8.89 (1H, s, CH). ¹³C NMR (100 MHZ; CD₃OD): δ 28.2, 31.4, 39.0, 64.9, 80.8, 120.3, 120.9, 122.5, 125.6, 126.1, 126.3, 128.9, 129.2, 135.6, 138.7, 140.1, 140.5, 141.5, 142.1, 143.1, 166.3, 181.2. ESI-HRMS (m/z): [M+H]⁺ calcd for C₂₉H₃₀FN₂O₄, 489.2184; found, 489.2138.

Methyl (E)-3-(3-((E)-but-2-en-1-yl)-5-((4-methylbenzyl)carbamoyl)phenyl)acrylate (4z): A transparent oil (75%). ¹H NMR (400 MHz; CDCl₃): δ 1.72 (3H, s, CH₃), 2.34 (3H, s, CH₃), 2.96 (2H, t, J=6.9 Hz, CH₂), 3.76 (2H, d, J=6.6 Hz, CH₂), 3.89 (3H, d, s, CH₃), 5.09-5.15 (1H, m, CH), 5.89-5.99 (1H, m, CH), 6.40 (1H, d, J=15.9 Hz, CH), 7.25-7.30 (2H, m, ArCH), 7.34-7.38 (2H, m, ArCH), 7.44 (1H, s, ArCH), 7.52 (1H, s, ArCH), 7.56 (1H, d, J=16.0 Hz, CH), 7.63 (1H, s, ArCH). ¹³C NMR (100 MHz; CDCl₃,): δ 28.1, 35.6, 39.8, 41.7, 80.7, 116.9, 121.3, 126.6, 128.5, 128.7, 128.8, 129.6, 130.8, 135.2, 135.5, 136.2, 138.8, 141.3, 142.4, 165.9, 167.0.

(E)-3-(3-((E)-But-2-en-1-yl)-5-((4-methylbenzyl)carbamoyl)phenyl)acrylic acid (A1-z): A white solid (83%). ¹H NMR (400 MHZ; CD₃OD): δ 1.72 (3H, s, CH₃), 2.34 (3H, s, CH₃), 2.97 (2H, t, J=6.9 Hz, CH₂), 3.77 (2H, d, J=6.6 Hz, CH₂), 5.09-5.15 (1H, m, CH), 5.89-5.99 (1H, m, CH), 6.40 (1H, d, J=15.9 Hz, CH), 7.25-7.30 (2H, m, ArCH), 7.34-7.38 (2H, m, ArCH), 7.44 (1H, s, ArCH), 7.52 (1H, s, ArCH), 7.56 (1H, d, J=16.0 Hz, CH), 7.63 (1H, s, ArCH). ¹³C NMR (100 MHz; CD₃OD): δ 28.1, 35.6, 39.8, 41.7, 80.7, 116.9, 121.3, 126.6, 128.5, 128.7, 128.8, 129.6, 130.8, 135.2, 135.5, 136.2, 138.8, 141.3, 142.4, 165.9, 167.0. HRMS-ESI (m/z): [M+H]⁺ calcd for C₂₂H₂₄NO₃, 350.1756; found, 350.1762.

Methyl (E)-3-(3-((Z)-but-2-en-1-yl)-5-((4-methylbenzyl)carbamoyl)phenyl)acrylate (4aa): A transparent oil (75%). ¹H NMR (400 MHZ; CDCl₃): δ 1.77 (3H, s, CH₃), 2.34 (3H, s, CH₃), 2.96 (2H, t, J=6.9 Hz, CH₂), 3.76 (2H, d, J=6.6 Hz, CH₂), 3.89 (3H, d, s, CH₃), 5.09-5.15 (1H, m, CH), 5.89-5.99 (1H, m, CH), 6.40 (1H, d, J=15.9 Hz, CH), 7.25-7.30 (2H, m, ArCH), 7.34-7.38 (2H, m, ArCH), 7.44 (1H, s, ArCH), 7.52 (1H, s, ArCH), 7.56 (1H, d, J=16.0 Hz, CH), 7.63 (1H, s, ArCH). ¹³C NMR (100 MHZ; CDCl₃): δ 28.1, 35.6, 39.8, 41.7, 80.7, 116.9, 121.3, 126.6, 128.5, 128.7, 128.8, 129.6, 130.8, 135.2, 135.5, 136.2, 138.8, 141.3, 142.4, 165.9, 167.0.

(E)-3-(3-((Z)-but-2-en-1-yl)-5-((4-methylbenzyl)carbamoyl)phenyl)acrylic acid (A1-aa): White solid (87%). ¹H NMR (400 MHZ; CD₃OD): δ 1.77 (3H, s, CH₃), 2.34 (3H, s, CH₃), 2.96 (2H, t, J=6.9 Hz, CH₂), 3.76 (2H, d, J=6.6 Hz, CH₂), 5.09-5.15 (1H, m, CH), 5.89-5.99 (1H, m, CH), 6.40 (1H, d, J=15.9 Hz, CH), 7.25-7.30 (2H, m, ArCH), 7.34-7.38 (2H, m, ArCH), 7.44 (1H, s, ArCH), 7.52 (1H, s, ArCH), 7.56 (1H, d, J=16.0 Hz, CH), 7.63 (1H, s, ArCH). ¹³C NMR (100 MHz; CD₃OD): δ 28.1, 35.6, 39.8, 41.7, 80.7, 116.9, 121.3, 126.6, 128.5, 128.7, 128.8, 129.6, 130.8, 135.2, 135.5, 136.2, 138.8, 141.3, 142.4, 165.9, 167.0. HRMS (ESI): (m/z): [M+H]⁺ calcd for C₂₂H₂₄NO₃, 350.1756; found, 350.1769.

Methyl (E)-3-(4′-(cyclopropylethynyl)-5-(2-(p-tolyl) acetamido)-[1,1′-biphenyl]-3-yl)acrylate (4bb): A transparent oil (79%). ¹H NMR (400 MHZ; CDCl₃): δ 0.89 (2H, m, J=7.4 Hz, CH₂), 1.28 (3H, s, J=7.4 Hz, CH₂ and CH), 2.32 (3H, s, CH₃), 3.89 (3H, s, CH₃), 4.64 (2H, d, J=7.4 Hz, CH₂), 6.54 (1H, s, CH), 7.15-7.22 (2H, d, J=7.8 Hz, ArCH), 7.34 (2H, d, J=8.0 Hz, ArCH), 7.56 (1H, d, J=8.0, CH), 7.73 (1H, s, ArCH), 7.83 (1H, s, ArCH), 7.98 (1H, d, J=8.0, ArCH). ¹³C NMR (100 MHz; CDCl₃): δ 16.5, 24.4, 33.4, 42.4, 119.3, 119.3, 120.7, 122.0, 123.8, 128.7, 128.8, 130.7, 133.1, 134.8, 134.9, 138.1, 143.3, 143.8, 148.1, 168.2.

(E)-3-(3-(Cyclopropylethynyl)-5-((4-methylbenzyl)carbamoyl)phenyl)acrylic acid (A1-bb): A white solid (71%). ¹H NMR (400 MHZ; CD₃OD): δ 0.89 (2H, m, J=7.4 Hz, CH₂), 1.28 (3H, s, J=7.4 Hz, CH₂ and CH), 2.32 (3H, s, CH₃), 4.64 (2H, d, J=7.4 Hz, CH₂), 6.54 (1H, s, CH), 7.15 (2H, d, J=7.8 Hz, ArCH), 7.24 (2H, d, J=8.0 Hz, ArCH), 7.56 (1H, d, J=8.0, CH), 7.78 (1H, s, ArCH), 7.83 (1H, s, ArCH), 7.98 (1H, d, J=8.0, ArCH). ¹³C NMR (100 MHZ; CD₃OD): δ 16.5, 24.4, 33.4, 42.4, 119.3, 119.3, 120.7, 122.0, 123.8, 128.7, 128.8, 130.7, 133.1, 134.8, 134.9, 138.1, 143.3, 143.8, 148.1, 168.2. HRMS-ESI (m/z): [M+H]⁺ calcd for C₂₃H₂₂NO₃, 360.1600; found, 360.3633.

(E)-3-(3-((4-Methylbenzyl)carbamoyl)-5-((3-methylbut-2-en-1-yl)oxy)phenyl)acrylic acid (10). A 250 mL round bottom flask was charged with 3-bromo-5-hydroxybenzoic acid 6 (5 g, 23.0 mmol), PPh₃ (1.21 g, 4.6 mmol), and Pd(OAc)₂ (258.63 mg, 1.2 mmol) and the flask was flushed with N₂ and 100 mL toluene was added to the flask, followed by the addition of Et₃N (9.6 mL, 69.1 mmol) and methyl acrylate (3.1 mL, 34.6 mmol). The reaction mixture was heated at 110° C. overnight and the contents were filtered on celite, the filtrate was evaporated under vacuum, and the crude product was purified via column chromatography (40% EtOAc in Hexanes) to obtain (E)-3-hydroxy-5-(3-methoxy-3-oxoprop-1-en-1-yl)benzoic acid 7 in 68% yield. To a solution of compound 7 (5.1 g, 22.9 mmol) in 50 mL DCM:DMF (1:1), EDC (4.28 g, 27.5 mmol) and HOBt (3.72 g, 27.5 mmol) was added the reaction mixture was stirred at room temperature for 15 minutes, followed by the addition of p-tolylmethanamine (3.5 mL, 27.5 mmol) and Et₃N (9.6 mL, 68.9 mmol). The reaction was stirred at room temperature overnight and the contents were evaporate under vacuum and resuspended in water, filtered, and washed with water and hexanes. The solid obtained was purified via column chromatography (50% EtOAc:Hexanes) to get methyl (E)-3-(3-hydroxy-5-((4-methylbenzyl)carbamoyl)phenyl)acrylate 8 in 79% yield. To a solution of 8 (500 mg, 1.54 mmol) in 20 mL acetonitrile, 1-bromo-3-methylbut-2-ene (0.36 mL, 3.1 mmol) was added, followed by K₂CO₃ (424.8 mg, 3.1 mmol). The reaction was stirred at reflux overnight, allowed to cool and was filtered and evaporated under vacuum. The crude product was purified via column chromatography (50% EtOAc:Hexanes) to obtain methyl (E)-3-(3-((4-methylbenzyl)carbamoyl)-5-((3-methylbut-2-en-1-yl)oxy)phenyl)acrylate 9. To a solution of compound 9 in 10 mL MeOH-THF (1:1), NaOH (91.5 mg, 2.3 mmol) was added and the reaction was stirred at 60° C. for 4 hours. The reaction was quenched in 6N HCl and the resulting solid was filtered and washed with water and hexanes. The crude product was purified via column chromatography using MeOH-DCM to obtain (E)-3-(3-((4-methylbenzyl)carbamoyl)-5-((3-methylbut-2-en-1-yl)oxy)phenyl)acrylic acid 10 as a white solid (312 mg, Yield 52%). ¹H NMR (400 MHZ, DMSO-de): δ 12.52 (1H, s, OH), 9.08 (1H, t, J=4.9 Hz, NH), 7.81 (1H, s, ArCH), 7.62-7.57 (1H, m, ArCH), 7.49 (1H, s, ArCH), 7.42 (1H, s, ArCH), 7.23 (2H, d, J=6.3 Hz, ArCH), 7.14 (2H, d, J=6.7 Hz, ArCH), 6.65 (1H, dd, J=2.3, 16.0 Hz, CH), 5.45 (1H, t, J=5.4 Hz, CH), 4.62 (2H, d, J=6.5 Hz, CH₂), 4.45 (2H, d, J=5.2 Hz, CH₂), 2.27 (3H, s, CH₃), 1.74 (6H, d, J=8.9 Hz, CH₃). ¹³C NMR (100 MHZ, DMSO-d₆): δ 167.9, 165.7, 159.3, 143.7, 138.0, 136.9, 136.5, 136.3, 136.2, 129.3, 127.8, 121.0, 120.0, 119.2, 117.6, 115.7, 65.2, 42.9, 25.9, 21.1, 18.5. HRMS-ESI (m/z): [M+H]⁺ calcd for C₂₃H₂₅NO₄, 380.1857; found, 380.1846.

(E)-3-(3-Bromo-5-((4-methylbenzyl)carbamoyl)phenyl)acrylic acid (11). To a stirred solution of ester 3 (0.33 g, 0.85 mmol) in a mixture of THF/MeOH (4:1, 20 mL) was added aqueous 1 N NaOH (2.5 mL, 2.5 mmol). The mixture was stirred at 60° C. for 3 h. The solvent was evaporated in vacuo and the pH of the reaction mixture was adjusted to 2-4 with 1 N HCl. The mixture was extracted with CH₂Cl₂ (3×50 mL). The combined organic layers were washed with brine (40 mL), dried over anhydrous Na₂SO₄, filtered, and evaporated to dryness under reduced pressure. The residue was purified by silica gel column chromatography using CH₂Cl₂/MeOH as eluent to afford the titled compound as a white solid (0.25 g, Yield 78%). ¹H NMR (500 MHZ, DMSO-d₆): δ 12.58 (1H, br s, OH), 9.13 (1H, t, J=6.0 Hz, NH), 8.22 (1H, s, ArCH), 8.10 (1H, s, ArCH), 8.06 (1H, s, ArCH), 7.61 (1H, d, J=16.0 Hz, CH), 7.22 (2H, d, J=8.0 Hz, ArCH), 7.14 (2H, d, J=8.0 Hz, ArCH), 7.69 (1H, d, J=16.0 Hz, CH), 4.45 (2H, d, J=6.0 Hz, CH₂), 2.28 (3H, s, CH₃). ¹³C NMR (125 MHz, DMSO-d₆): δ 167.68, 164.57, 141.98, 137.36, 137.16, 136.60, 136.41, 133.94, 131.73, 129.34, 127.83, 125.66, 122.80, 122.50, 43.06, 21.14. HRMS-ESI (m/z): [M+H]⁺ calcd for C₁₈H₁₇BrNO₃, 374.0386; found, 374.0390.

(E)-3-(3-Amino-3-oxoprop-1-en-1-yl)-5-bromo-N-(4-methylbenzyl)benzamide (12). To a stirred solution of acid 11 (0.1 g, 0.27 mmol) in an anhydrous THF (8 mL) was added thionyl chloride (78 μL, 1.07 mmol). The mixture was stirred at 70° C. for 3 h. The solvent was evaporated in vacuo and the residue was dissolved in anhydrous CH₂Cl₂ (10 mL) and treated with 30% aq. ammonium hydroxide solution (140 μL, 1.07 mmol) at 0° C. The pH of the reaction mixture was adjusted to 5-7 with 1N HCl and extracted with CH₂Cl₂ (3×30 mL). The combined organic layers were dried over anhydrous Na₂SO₄, filtered, and evaporated to dryness under reduced pressure to afford a crude compound. The residue was purified by silica gel column chromatography using CH₂Cl₂/MeOH as eluent to afford the titled compound as a white solid (65 mg, Yield 65%). ¹H NMR (500 MHZ, CD₃OD): δ 8.12 (1H, s, ArCH), 7.94 (2H, d, J=1.5 Hz, ArCH), 7.56 (1H, d, J=16.0 Hz, CH), 7.36 (2H, d, J=8.5 Hz, ArCH), 7.27 (2H, d, J=8.5 Hz, ArCH), 6.84 (1H, d, J=16.0 Hz, 1H), 4.66 (2H, s, CH₂), 2.38 (3H, s, CH₃). ¹³C NMR (125 MHZ, CD₃OD): δ 168.30, 162.85, 138.19, 138.13, 137.71, 134.47, 131.64, 131.20, 131.05, 129.33, 127.71, 126.06, 124.14, 123.02, 46.4, 19.78. HRMS-ESI (m/z): [M+H]⁺ calcd for C₁₈H₁₈BrN₂O₂, 373.0546; found, 373.0748.

(E)-5-(3-Amino-3-oxoprop-1-en-1-yl)-N-(4-methylbenzyl)-4′-phenoxy-[1,1′-biphenyl]-3-carboxamide (13). To a stirred solution of acid A1-n (0.05 g, 0.1 mmol) in THF (5 mL) was added thionyl chloride (30 μL, 0.4 mmol). The mixture was stirred at 70° C. for 3 h. After cooled to room temperature, the solvent was evaporated in vacuo. The residue was dissolved in anhydrous CH₂Cl₂ (10 mL) was added 30% aqueous ammonium hydroxide (60 μL, 0.4 mmol) at 0° C. The reaction mixture was stirred at 0° C. for 1 h, and the pH of the reaction mixture was adjusted to 5-7 with 1N HCl. The mixture was extracted with CH₂Cl₂ (3×25 mL). The combined organic layers were dried over anhydrous Na₂SO₄, filtered, and evaporated to dryness under reduced pressure. The residue was purified by silica gel column chromatography using CH₂Cl₂/MeOH as eluent to afford the titled compound as a white solid (28 mg, Yield 56%). ¹H NMR (500 MHZ, CDCl₃): δ 7.88 (1H, s, ArCH), 7.84 (1H, s, ArCH), 7.55 (1H, s, ArCH), 7.52 (1H, d, J=16.0 Hz, CH), 7.43 (1H, br s, NH), 7.39 (2H, d, J=8.5 Hz, ArCH), 7.32 (2H, t, J=8.0 Hz, ArCH), 7.21 (2H, d, J=7.5 Hz, ArCH), 7.11 (1H, t, J=7.5 Hz, ArCH), 7.07 (2H, d, J=7.5 Hz, ArCH), 6.99 (2H, d, J=8.0 Hz, ArCH), 6.96 (2H, d, J=8.0 Hz, ArCH), 6.52 (1H, d, J=16.0 Hz, CH), 6.43 (1H, br s, NH), 6.24 (1H, br s, NH), 4.55 (2H, d, J=4.5 Hz, CH₂), 2.26 (3H, s, CH₃). ¹³C NMR (125 MHZ, CDCl₃): δ 168.00, 167.13, 157.50, 156.77, 141.44, 141.10, 137.27, 135.64, 135.45, 135.18, 134.29, 129.87, 129.42, 128.43, 127.99, 126.78, 126.78, 124.52, 123.65, 119.14, 118.99, 43.97, 21.09. HRMS-ESI (m/z): [M+H]⁺ calcd for C₃₀H₂₇N₂O₃, 463.2016; found, 463.2018.

(E)-3-Bromo-5-(3-(dimethylamino)-3-oxoprop-1-en-1-yl)-N-(4-methylbenzyl)benzamide (14). To a stirred suspension of 3-bromo-5-iodo-N-(4-methylbenzyl)benzamide 2 (0.15 g, 0.35 mmol) in anhydrous toluene (15 mL) were added N,N-dimethylacrylamide (50 μL, 0.45 mmol), Pd(OAc)₂ (8.0 mg, 0.035 mmol), and triethylamine (0.1 mL, 0.7 mmol). The mixture was heated at 90° C. for 12 h under N₂ atmosphere. After cooled to room temperature, the reaction mixture was filtered through celite and washed with EtOAc (50 mL). The filtrate was washed with 1N HCl (25 mL), and brine (25 mL). The organic layer was dried over anhydrous Na₂SO₄, filtered, and evaporated to dryness under reduced pressure. The residue was purified by Biotage select flash column chromatography using EtOAc/Hexanes as eluent to afford the titled compound as an off-white solid (90 mg, Yield 50%). ¹H NMR (400 MHz, CD₃OD): δ 8.01 (1H, s, ArCH), 7.96 (1H, s, ArCH), 7.86 (1H, s, ArCH), 7.44 (1H, d, J=15.2 Hz, CH), 7.21 (2H, d, J=8.0 Hz, ArCH), 7.15 (1H, d, J=15.2 Hz, CH), 7.10 (2H, d, J=8.0 Hz, ArCH), 4.51 (2H, s, CH₂), 3.17 (3H, s, NCH₃), 3.01 (3H, s, NCH₃), 2.28 (3H, s, CH₃). ¹³C NMR (100 MHZ, MeOH-d₄): δ 165.26, 164.70, 138.10, 136.12, 135.12, 135.10, 133.91, 131.58, 129.44, 127.31, 125.80, 123.44, 121.17, 118.54, 41.65, 35.03, 33.44, 18.35. HRMS-ESI (m/z): [M+H]⁺ calcd for C₂₀H₂₂BrN₂O₂, 401.0859; found, 401.0858.

(E)-3-Bromo-5-(2-cyanovinyl)-N-(4-methylbenzyl)benzamide (15). To a stirred suspension of 3-bromo-5-iodo-N-(4-methylbenzyl)benzamide 2 (1.5 g, 3.48 mmol) in anhydrous toluene (30 mL) were added acrylonitrile (0.34 mL, 5.25 mmol), Pd(OAc)₂ (78 mg, 0.35 mmol), triphenylphosphine (91 mg, 0.35 mmol) and triethylamine (1.46 mL, 10.5 mmol). The mixture was heated at 90° C. for 12 under N₂ atmosphere. After cooled to room temperature, the reaction mixture was filtered through celite and washed with EtOAc (50 mL). The filtrate was washed with 1N HCl (50 mL), and brine (50 mL). The organic layer was dried over anhydrous Na₂SO₄, filtered, and evaporated to dryness under reduced pressure. The residue was purified by silica gel column chromatography using EtOAc/Hexanes as eluent to afford the titled compound as a white solid (0.62 g, Yield 50%). ¹H NMR (400 MHZ, CDCl₃): δ 7.91 (1H, d, J=1.2 Hz, ArCH), 7.84 (1H, d, J=1.2 Hz, ArCH), 7.71 (1H, d, J=1.2 Hz, ArCH), 7.35 (1H, d, J=16.8 Hz, CH), 7.26 (2H, d, J=7.6 Hz, ArCH), 7.20 (2H, d, J=7.6 Hz, ArCH), 6.41 (1H, br s, NH), 5.97 (1H, dd, J=16.8, 1.2 Hz, CH), 4.61 (2H, d, J=5.2 Hz, CH₂), 2.38 (3H, s, CH₃). ¹³C NMR (125 MHZ, CDCl₃): 164.76, 147.88, 137.80, 137.22, 135.80, 134.42, 132.68, 131.88, 129.61, 128.09, 124.89, 123.47, 117.17, 99.45, 44.27, 21.14. HRMS-ESI (m/z): [M+H]+ calcd for C₁₈H₁₆BrN₂O, 355.0441; found, 355.0444.

(E)-5-(2-Cyanovinyl)-N-(4-methylbenzyl)-4′-phenoxy-[1,1′-biphenyl]-3-carboxamide (16a). To a stirred solution of aryl bromide 15 (0.20 g, 0.56 mmol) and (4-phenoxyphenyl)boronic acid (0.24 g, 1.1 mmol) in anhydrous DMF (10 mL) were added Pd(dppf)Cl₂·CH₂Cl₂ (46 mg, 0.056 mmol), and CS₂CO₃ (0.55 g, 1.7 mmol). The mixture was stirred at 100° C. for 12 h under N₂ atmosphere. After cooled to room temperature, the reaction mixture was filtered through celite and washed with EtOAc (50 mL). The filtrate was washed with 1 N HCl (30 mL), and brine (50 mL). The organic layer was dried over anhydrous Na₂SO₄, filtered, and evaporated to dryness under reduced pressure. The residue was purified by silica gel column chromatography using EtOAc/Hexanes as eluent to afford the titled compound as a white solid (0.17 g, Yield 67%). ¹H NMR (500 MHZ, CDCl₃): δ 7.98 (1H, s, ArCH), 7.86 (1H, s, ArCH), 7.73 (1H, s, ArCH), 7.55 (2H, d, J=8.5 Hz, ArCH), 7.48 (1H, d, J=17.0 Hz, CH), 7.41-7.38 (3H, m, ArCH), 7.29 (2H, d, J=8.0 Hz, ArCH), 7.20 (2H, d, J=8.0 Hz, ArCH), 7.18 (1H, t, J=7.0 Hz, ArCH), 7.11 (2H, d, J=8.5 Hz, ArCH), 7.08 (2H, d, J=8.5 Hz, ArCH), 6.48 (1H, br s, NH), 6.03 (1H, d, J=17.0 Hz, CH), 4.65 (2H, d, J=5.5 Hz, CH₂), 2.38 (3H, s, CH₃). ¹³C NMR (125 MHZ, CDCl₃): δ 166.24, 157.94, 156.68, 149.47, 142.27, 137.68, 136.13, 134.73, 134.53, 133.92, 129.91, 129.58, 128.58, 128.10, 127.70, 124.31, 123.80, 119.27, 119.10, 117.67, 98.19, 44.20, 21.13. HRMS-ESI (m/z): [M+H]⁺ calcd for C₃₀H₂₅NO₂, 445.1911; found, 445.1911.

(E)-3-(2-Cyanovinyl)-N-(4-methylbenzyl)-5-(3-methylbut-2-en-1-yl)benzamide (16b). To a stirred solution of aryl bromide 15 (0.3 g, 0.84 mmol) in anhydrous DMF (10 mL) were added prenyltributyltin (0.55 mL, 1.63 mmol), and Pd(Ph₃)₄ (98 mg, 0.084 mmol). The mixture was heated at 100° C. for 24 h under N₂ atmosphere. After cooled to room temperature, the reaction mixture was filtered through celite and washed with EtOAc (50 mL. The filtrate was washed with a 10% aqueous KF solution (2×50 mL), and 1 N NaOH 3 times (50 mL). The organic layer was dried over anhydrous Na₂SO₄, filtered, and evaporated to dryness under reduced pressure. The residue was purified by silica gel column chromatography using EtOAc/Hexanes as eluent to afford the titled compound as a white solid (0.14 g, Yield 48%). ¹H NMR (500 MHZ, CDCl₃): δ 7.74 (1H, s, ArCH), 7.68 (1H, s, ArCH), 7.34 (1H, s, ArCH), 7.23 (1H, d, J=16.5 Hz, CH), 7.22 (2H, d, J=7.5 Hz, ArCH), 7.14 (3H, m, ArCH), 5.86 (1H, d, J=16.5 Hz, CH), 5.28 (1H, t, J=7.5 Hz, NH), 4.55 (2H, d, J=6.0 Hz, CH₂), 3.37 (2H, d, J=7.5 Hz, CH₂), 2.34 (3H, s, CH₃), 1.77 (3H, s, CH₃), 1.72 (3H, s, CH₃). ¹³C NMR (125 MHZ, CDCl₃): δ 166.65, 149.86, 143.55, 137.28, 135.48, 135.09, 134.08, 133.92, 130.20, 129.72, 129.41, 127.89, 123.53, 121.70, 117.98, 97.27, 43.89, 34.00, 25.77, 21.12, 17.96. HRMS-ESI (m/z): [M+H]⁺ calcd for C₂₃H₂₅N₂O, 345.1961; found, 345.1965.

(R)-3-Bromo-5-iodo-N-(1-phenylpropyl)benzamide (17). A mixture of 3-bromo-5-iodobenzoic acid 1 (3.2 g, 9.79 mmol) and thionyl chloride (15 mL) was refluxed for 3 h. After cooled to room temperature, the solvent was evaporated to dryness under reduced pressure. The residue was dissolved in anhydrous CH₂Cl₂ (30 mL) and cooled to 0°, added triethylamine (4.1 mL, 29.7 mmol), and (R)-1-phenylpropan-1-amine (1.6 g, 11.88 mmol). The reaction mixture was stirred at room temperature for 8 h. The reaction mixture was washed with aqueous saturated NaHCO₃ (2×50 mL) and, 1 N HCl (2×50 mL). The CH₂Cl₂ solution was dried over anhydrous Na₂SO₄, filtered, and evaporated to dryness under reduced pressure to afford the tilted compound as a white solid (3.52 g, Yield 81%). ¹H NMR (500 MHZ, CDCl₃+CD₃OD): δ 7.98 (1H, d, J=4.0 Hz, ArCH), 7.86 (1H, d, J=3.5 Hz, ArCH), 7.82 (1H, d, J=3.5 Hz, ArCH), 7.28-7.25 (4H, m, ArCH), 7.21-7.18 (1H, m, ArCH), 4.91 (1H, t, J=7.5 Hz, CH), 1.90-1.78 (2H, m, CH₂), 0.90-0.87 (3H, m, CH₃). ¹³C NMR (125 MHZ, CDCl₃+CD₃OD): δ 164.58, 142.06, 141.92, 137.88, 134.91, 129.76, 128.53, 127.31, 126.69, 122.93, 94.21, 55.87, 28.77, 10.97.

(R,E)-3-Bromo-5-(2-methoxyvinyl)-N-(1-phenylpropyl)benzamide (18). To a stirred suspension of 17 (1.0 g, 2.25 mmol) in anhydrous toluene (30 mL) were added methyl acrylate (260 μL, 2.92 mmol), Pd(OAc)₂ (50 mg, 0.23 mmol), triphenylphosphine (60 mg, 0.23 mmol), and triethylamine (1.0 mL, 6.75 mmol). The mixture was stirred at 90° C. for 12 h. After cooled to room temperature, the reaction mixture was filtered through celite and washed with EtOAc (50 mL). The filtrate was washed with aqueous 1 N HCl (50 mL) and brine (50 mL). The aqueous layer was extracted with EtOAc (2×50 mL). The combined organic layers were dried over anhydrous Na₂SO₄, filtered, and evaporated to dryness under reduced pressure. The residue was purified by silica gel column chromatography using EtOAc/Hexanes as eluent to afford the titled compound as off-white solid (0.58 g, Yield 64%). ¹H NMR (500 MHZ, CDCl₃): δ 7.84 (1H, s, ArCH), 7.77 (1H, s, ArCH), 7.62 (1H, s, ArCH), 7.46 (1H, d, J=16.0 Hz, CH), 7.39 (1H, br d, J=8.0 Hz, NH), 7.33-7.28 (4H, m, ArCH), 7.25-7.22 (1H, m, ArCH), 6.36 (1H, d, J=16.0 Hz, CH), 5.01 (1H, m, CH), 3.78 (3H, s, OCH₃), 1.98-1.86 (2H, m, CH₂), 0.93 (3H, t, J=7.5 Hz, CH₃). ¹³C NMR (125 MHZ, CDCl₃): δ 166.71, 165.19, 142.17, 141.92, 137.21, 136.45, 133.08, 131.55, 128.64, 127.43, 126.78, 125.45, 123.01, 120.18, 55.94, 51.94, 28.99, 11.05.

Methyl (R,E)-3-(4′-isopropyl-5-((1-phenylpropyl)carbamoyl)-[1,1′-biphenyl]-3-yl)acrylate (19a). To a stirred solution of 18 (0.20 g, 0.50 mmol) and (4-isopropylphenyl)boronic acid (0.16 g, 1.0 mmol) in anhydrous DMF (10 mL) were added Pd(dppf)Cl₂·CH₂Cl₂ (40 mg, 0.05 mmol) and CS₂CO₃ (0.50 g, 1.5 mmol). The mixture was stirred at 100° C. for 12 under N₂ atmosphere. After cooled to room temperature, the reaction mixture was filtered through celite and washed with EtOAc (50 mL). The filtrate was washed with 1 N HCl (30 mL) and brine 50 (mL). The aqueous layer was extracted with EtOAc (2×50 mL), and the combined organic layers were dried over Na₂SO₄, filtered, and evaporated to dryness under reduced pressure. The residue was purified by silica gel column chromatography using EtOAc/Hexanes as eluent to afford the titled compound as a white solid (0.16 g, Yield 72%). ¹H NMR (500 MHZ, CDCl₃) δ 7.98 (1H, s, ArCH), 7.86 (1H, s, ArCH), 7.79 (1H, s, ArCH), 7.73 (1H, d, J=16.0 Hz, CH), 7.51 (2H, d, J=8.5 Hz, ArCH), 7.40-7.28 (7H, m, ArCH), 6.72 (1H, d, J=8.0 Hz, NH), 6.53 (1H, d, J=16.0 Hz, CH), 5.13 (1H, m, CH), 3.82 (3H, s, OCH₃), 2.98 (1H, m, CH), 2.04-1.94 (2H, m, CH₂), 1.31 (6H, d, J=6.5 Hz, (CH₃) 2), 0.99 (3H, t, J=7.5 Hz, CH₃). ¹³C NMR (100 MHZ, CDCl₃): δ 166.69, 148.93, 142.45, 141.99, 141.44, 137.51, 135.97, 133.06, 132.84, 129.55, 129.03, 128.76, 127.59, 127.48, 127.28, 127.08, 126.81, 124.93, 124.46, 55.68, 33.86, 29.72, 29.14, 23.99, 10.98.

Methyl (R,E)-3-(4′-phenoxy-5-((1-phenylpropyl)carbamoyl)-[1,1′-biphenyl]-3-yl)acrylate (19b). To a stirred solution of 18 (0.20 g, 0.50 mmol) and (4-phenoxyphenyl)boronic acid (0.21 g, 1.0 mmol) in anhydrous DMF (10 mL) were added Pd(dppf)Cl₂·CH₂Cl₂ (40 mg, 0.05 mmol) and CS₂CO₃ (0.50 g, 1.5 mmol). The mixture was stirred at 100° C. for 12 under N₂ atmosphere. After cooled to room temperature, the reaction mixture was filtered through celite and washed with EtOAc (50 mL). The filtrate was washed with 1 N HCl (30 mL) and brine 50 (mL). The aqueous layer was extracted with EtOAc (2×50 mL), and the combined organic layers were dried over Na₂SO₄, filtered, and evaporated to dryness under reduced pressure. The residue was purified by silica gel column chromatography using EtOAc/Hexanes as eluent to afford the titled compound as a white solid (0.21 g, Yield 86%). ¹H NMR (500 MHZ, CDCl₃): δ 7.97 (1H, s, ArCH), 7.85 (1H, s, ArCH), 7.77 (1H, s, ArCH), 7.72 (1H, d, J=16.0 Hz, CH), 7.52 (2H, d, J=9.0 Hz, ArCH), 7.41-7.35 (6H, m, ArCH), 7.29 (1H, t, J=7.0 Hz, ArCH), 7.17 (1H, t, J=7.5 Hz, ArCH), 7.09-707 (4H, m, ArCH), 6.72 (1H, d, J=8.0 Hz, NH), 6.52 (1H, d, J=16.0 Hz, CH), 5.13 (1H, m, CH), 3.82 (3H, s, OCH₃), 2.06-1.94 (2H, m, CH₂), 0.99 (3H, t, J=7.5 Hz, CH₃). ¹³C NMR (125 MHZ, CDCl₃): δ 167.07, 166.25, 157.68, 156.79, 143.72, 141.93, 141.79, 136.13, 135.29, 134.34, 129.89, 129.11, 128.76, 128.53, 127.53, 127.23, 126.79, 124.81, 123.70, 119.32, 119.22, 119.05, 55.70, 51.85, 29.08, 10.96.

(R,E)-3-(4′-Isopropyl-5-((1-phenylpropyl)carbamoyl)-[1,1′-biphenyl]-3-yl)acrylic acid (20a). To a stirred solution of methyl ester 19a (0.13 g, 0.29 mmol) in a mixture of THE/MeOH (10 mL, 4:1) was added aqueous 1 N NaOH (0.9 mL 0.88 mmol). The mixture was stirred at 60° C. for 3 h. The solvent was evaporated in vacuo and the pH of the reaction mixture was adjusted to 2-4 with 1 N HCl. The mixture was extracted with CH₂Cl₂ (3×40 mL) and washed with brine (30 mL). The combined organic layers were dried over anhydrous Na₂SO₄, filtered, and evaporated to dryness under reduced pressure. The residue was purified by silica gel column chromatography using CH₂Cl₂/MeOH as eluent to afford the titled compound as a white solid (90 mg, Yield 72%). ¹H NMR (500 MHZ, CDCl₃+CD₃OD): δ 7.89 (1H, s, ArCH), 7.88 (1H, s, ArCH), 7.67 (1H, s, ArCH), 7.62 (1H, d, J=16.0 Hz, CH), 7.41 (2H, d, J=8.0 Hz, ArCH), 7.26-7.14 (6H, m, ArCH), 7.12 (1H, t, J=7.5 Hz, ArCH), 6.42 (1H, d, J=16.0 Hz, CH), 4.93 (1H, t, J=7.5 Hz, CH), 2.85-2.80 (1H, m, CH), 1.86-1.77 (2H, m, CH₂), 1.15 (6H, d, J=6.5 Hz, (CH₃) 2), 0.85 (3H, t, J=7.5 Hz, CH₃). ¹³C NMR (125 MHZ, CDCl₃+CD₃OD): δ 168.80, 167.01, 148.94, 144.24, 142.26, 142.12, 136.93, 135.71, 135.14, 129.31, 128.50, 127.46, 127.23, 126.97, 126.62, 124.78, 119.56, 55.65, 33.73, 28.85, 23.74, 10.85. HRMS-ESI (m/z): [M+H]⁺ calcd for C₂₈H₃₀NO₃, 428.2220; found, 428.2225.

(R,E)-3-(4′-Phenoxy-5-((1-phenylpropyl)carbamoyl)-[1,1′-biphenyl]-3-yl)acrylic acid (20b). To a stirred solution of methyl ester 19b (0.15 g, 0.31 mmol) in a mixture of THE/MeOH (10 mL, 4:1) was added aqueous 1 N NaOH (0.9 mL 0.92 mmol). The mixture was stirred at 60° C. for 3 h. The solvent was evaporated in vacuo and the pH of the reaction mixture was adjusted to 2-4 with 1 N HCl. The mixture was extracted with CH₂Cl₂ (3×40 mL) and washed with brine (30 mL). The organic layer was dried over anhydrous Na₂SO₄, filtered, and evaporated to dryness under reduced pressure. The residue was purified by silica gel column chromatography using CH₂Cl₂/MeOH as eluent to afford the titled compound as a white solid (0.12 g, Yield 86%). ¹H NMR (500 MHZ, CDCl₃+CD₃OD): δ 7.87 (1H, s, ArCH), 7.80 (1H, s, ArCH), 7.64 (1H, s, ArCH), 7.56 (1H, d, J=16.0 Hz, CH), 7.42 (2H, d, J=8.5 Hz, ArCH), 7.26-7.14 (6H, m, ArCH), 7.07 (1H, t, J=7.5 Hz, ArCH), 6.96 (1H, t, J=7.5 Hz, ArCH), 6.91-6.86 (4H, m, ArCH), 6.38 (1H, d, J=16.0 Hz, CH), 4.86 (1H, t, J=7.5 Hz, CH), 1.84-1.73 (2H, m, CH₂), 0.81 (3H, t, J=7.5 Hz, CH₃). ¹³C NMR (125 MHZ, CDCl₃+CD₃OD): δ 172.81, 171.22, 161.50, 160.72, 148.12, 146.33, 145.53, 139.75, 139.19, 138.42, 133.72, 133.11, 132.40, 131.46, 131.10, 130.60, 128.86, 127.53, 123.64, 123.00, 122.86, 59.79, 32.79, 14.86. HRMS-ESI (m/z): [M+H]⁺ calcd for C₃₁H₂₈NO₄, 478.2013; found, 478.2014.

Structure-Activity Relationship: The ability of compounds to inhibit AKR1C₃ in the first instance were determined by measuring the NADP+ dependent oxidation of S-tetralol catalyzed by recombinant enzyme as described herein. Compounds that showed potent inhibition (IC₅₀ values<100 nM) of AKR1C3 were counter-screened against AKR1C2 to determine selectivity.

AKR enzyme inhibition assay: (S)-(+)-1,2,3,4-tetrahydro-1-naphthol (S-tetralol) was purchased from Sigma-Aldrich (St. Louis, MO). Nicotinamide adenine dinucleotide (NAD⁺) and nicotinamide adenine dinucleotide phosphate (NADP⁺) were purchased from Roche Diagnostics (Indianapolis, IN). Homogeneous recombinant enzymes AKR1C1, AKR1C2, AKR1C3 and AKR1C4 were prepared and purified as previously described. The specific activities of the enzymes were as follows for the oxidation of S-tetralol: AKR1C1, AKR1C2 and AKR1C3 for the oxidation of S-tetralol are 1.6, 1.5 and 3.5 μmol min 1 mg 1, respectively; and for the oxidation of androsterone AKR1C4 had a specific activity of 0.32 μmoles/min/mg.

Assay of enzyme activity: The dehydrogenase activities of AKR1C1, AKR1C2, and AKR1C3 were determined by measuring the formation of NADH formation at 340 nm using Beckman DU640 spectrophotometer. A typical assay solution contained 100 mM potassium phosphate pH 7.0, 2.3 mM NAD⁺, 3.0 mM(S)-(+)-1,2,3,4-tetrahydro-1-naphthol (S-tetralol), 4% acetonitrile (v/v). The mixtures were incubated at 37° C. for 3 min followed by adding a serial dilution of AKR1C1, AKR1C2, or AKR1C3 solution to a final volume of 1 mL to initiate the reaction. For AKR1C4, 75 μM androsterone was substituted for S-tetralol. After continuously monitoring for 5 min, the increase in UV absorption using different concentrations of enzyme were recorded to calculate the initial velocity and determine enzyme specific activity.

IC₅₀ value determination: The inhibitory potency for each compound was represented by IC₅₀ value and measured as described before. The IC₅₀ value of coumarin analogues was determined by measuring their inhibition of the NADP⁺ dependent oxidation of S-tetralol catalyzed by AKR1C1, AKR1C2, and AKR1C3 in a 96-well plate format and the reaction measured fluorometrically with a (Exc/Emi, 340/460 nm) on a BIOTEK Synergy 2 Multimode plate reader. The assay mixture consisted of 100 mM phosphate buffer, pH 7.0, S-tetralol (in DMSO), inhibitor (in DMSO), 200 μM NADP⁺, and purified recombinant enzyme to give a total volume of 200 μL, and 4% DMSO. The concentration of S-tetralol used in the assays for each AKR1C isoform was equal to the Kₘ value for the respective enzyme so that IC₅₀ values could be directly compared assuming a competitive pattern of inhibition. The Kₘ value obtained for S-tetralol for AKR1C1, AKR1C2, AKR1C3 and AKR1C4 under the same experimental conditions are 8 μM, 15 μM, 165 μM, and 25 mM. The IC₅₀ value of each compound was acquired from a single experiment with each inhibitor concentration run in quadruplicate and directly calculated by fitting the inhibition data to an equation [y=(range)/[1+(I/IC₅₀)S]+background] using Grafit 5.0 software. In this equation, “range” is the fitted uninhibited value minus the “background”, and “S” is a slope factor. “I” is the concentration of inhibitor. The equation assumes that y falls with increasing “I”. The results are summarized in Tables 2-5.

TABLE 1
Compounds A1
(A1)

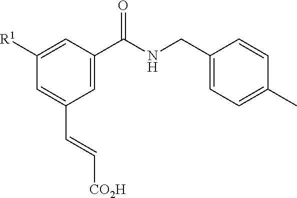

| Cpd | R¹ |
|---|---|
| Ref | 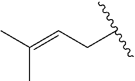 [SMILES: *CC=C(C)C] |
| A1-a | 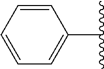 [SMILES: *c1ccccc1] |
| A1-b | 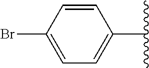 [SMILES: *c1ccc(Br)cc1] |
| A1-c | 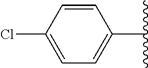 [SMILES: *c1ccc(Cl)cc1] |
| A1-d | 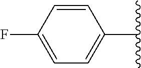 [SMILES: *c1ccc(F)cc1] |
| A1-e | 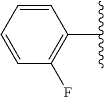 [SMILES: *c1ccccc1F] |
| A1-f | 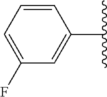 [SMILES: *c1cccc(F)c1] |
| A1-g | 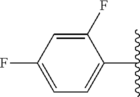 [SMILES: *c1ccc(F)cc1F] |
| A1-h | 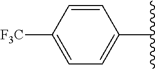 [SMILES: *c1ccc(C(F)(F)F)cc1] |
| A1-i | 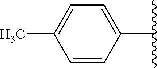 [SMILES: *c1ccc(C)cc1] |
| A1-1j | 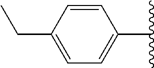 [SMILES: *c1ccc(CC)cc1] |
| A1-k | 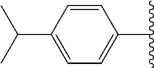 [SMILES: *c1ccc(C(C)C)cc1] |
| A1-l | 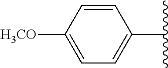 [SMILES: *c1ccc(OC)cc1] |
| A1-m | 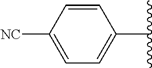 [SMILES: *c1ccc(C#N)cc1] |
| A-1n | 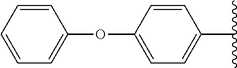 [SMILES: *c1ccc(Oc2ccccc2)cc1] |
| A1-o | 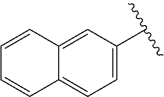 [SMILES: *c1ccc2ccccc2c1] |
| A1-p | 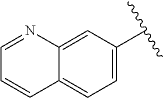 [SMILES: *c1ccc2cccnc2c1] |
| A1-q | 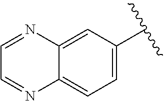 [SMILES: *c1ccc2nccnc2c1] |
| A1-r | 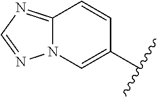 [SMILES: *c1ccc2ncnn2c1] |
| A1-s | 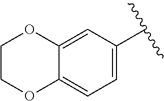 [SMILES: *c1ccc2c(c1)OCCO2] |
| A1-t | 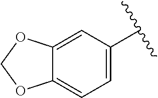 [SMILES: *c1ccc2c(c1)OCO2] |
| A1-u | 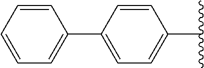 [SMILES: *c1ccc(-c2ccccc2)cc1] |
| A1-v | 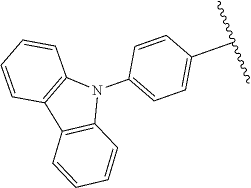 [SMILES: *c1ccc(-n2c3ccccc3c3ccccc32)cc1] |
| A1-w | 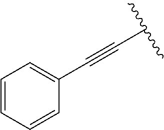 [SMILES: *C#Cc1ccccc1] |
| A1-x | 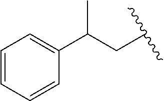 [SMILES: *CC(C)c1ccccc1] |
| A1-y | 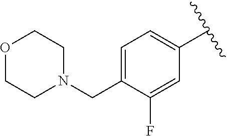 [SMILES: *c1ccc(CN2CCOCC2)c(F)c1] |
| A1-z | 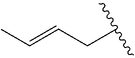 [SMILES: *C/C=C/C] |
| A1-aa | 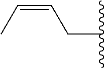 [SMILES: *C/C=C\C] |
| A1-bb | 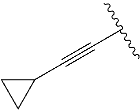 [SMILES: *C#CC1CC1] |
| 10 | 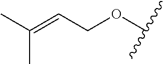 [SMILES: *OCC=C(C)C] |
| 11 | Br |

TABLE 2
Structure-activity relationships of prenyl analogs
for the inhibition of AKR1C3, 1C1, 1C2 and 1C4

| | IC₅₀ ± SD (μM) | | | | Selectivity |
|---|---|---|---|---|---|
| Cpd | AKR1C3 | AKR1C1 | AKR1C2 | AKR1C4 | 1C2:1C3 |
| Ref | 0.070 | >100 | >200 | 145 | >2850 |
| A1-a | 0.040 ± 0.0043 | >100 | >100 | 21 ± 2.5 | >2500 |
| A1-b | 0.090 ± 0.0035 | 107 ± 8.0 | 25 ± 7.0 | 6.5 ± 0.60 | 277 |
| A1-c | 0.060 ± 0.007 | >100 | 20 ± 9.0 | 6.0 ± 0.50 | 333 |
| A1-d | 0.06 ± 0.004 | >100 | 36 ± 4.0 | 8.0 ± 1.2 | 600 |
| A1-e | 0.070 ± 0.009 | >100 | >100 | 15.4 ± 1.70 | >1430 |
| A1-f | 0.050 ± 0.004 | >100 | 48 ± 16 | 16.4 ± 2.0 | 960 |
| A1-g | 0.13 ± 0.020 | N.D. | N.D. | N.D. | N.D. |
| A1-h | 0.20 ± 0.02 | N.D. | N.D. | N.D. | N.D. |
| A1-i | 0.10 ± 0.008 | 66 ± 17 | 16 ± 3.9 | 10 ± 1.72 | 160 |
| A1-1j | 0.19 ± 0.01 | N.D. | N.D. | N.D. | N.D. |
| A1-k | 0.19 ± 0.01 | N.D. | N.D. | N.D. | N.D. |
| A1-l | 0.17 ± 0.03 | N.D. | N.D. | N.D. | N.D. |
| A1-m | 0.17 ± 0.01 | N.D. | N.D. | N.D. | N.D. |
| A-1n | 0.094 ± 0.01 | N.D. | 4.8 ± 1.2 | N.D. | 51 |
| A1-o | 0.10 ± 0.011 | 78 ± 9.3 | 14 ± 4.1 | 6.1 ± 0.47 | 140 |
| A1-p | 0.11 ± 0.017 | 80 ± 13 | 12 ± 2.8 | 22 ± 2.30 | 109 |
| A1-q | 0.094 ± 0.01 | 70 ± 48.0 | 12.5 ± 2.1 | 31 ± 2.90 | 133 |
| A1-r | 0.051 ± 0.006 | >100 | 62 ± 21 | 43 ± 6.0 | 1216 |
| A1-s | 0.11 ± 0.011 | >100 | 22 ± 4.5 | 53 ± 7.1 | 200 |
| A1-t | 0.11 ± 0.021 | >100 | 36 ± 9.7 | 16 ± 1.6 | 327 |
| A1-u | 0.050 ± 0.004 | N.D. | 29 ± 4.8 | 2.4 ± 0.15 | 580 |
| A1-v | 0.14 ± 0.015 | 36 ± 5.5 | 31 ± 2.8 | 11 ± 3.1 | 221 |
| A1-w | 0.18 ± 0.019 | N.D. | 11 ± 3.1 | N.D. | 61 |
| A1-x | 0.88 ± 0.09 | N.D. | 156 ± 71 | N.D. | 177 |
| A1-y | 0.33 ± 0.11 | N.D. | 5.1 ± 0.6 | N.D. | 16 |
| A1-z | 0.11 ± 0.007 | >100 | 39 ± 9 | 70 ± 11 | 354 |
| A1-aa | 0.08 ± 0.004 | 81 ± 5.1 | 16 ± 2.5 | 25 ± 3.1 | 200 |
| A1-bb | 0.50 ± 0.04 | N.D. | 37 ± 9.6 | N.D. | 74 |
| 10 | 0.08 ± 0.01 | 52 ± 7.0 | 15 ± 2.4 | 31 ± 5.0 | 187 |
| 11 | 0.30 ± 0.01 | N.D. | >100 | 40 ± 1.3 | >333 |

Replacement of the prenyl chain of the reference AKR1C3 inhibitor with an unsubstituted phenyl ring (A1-a) afforded a compound with increased inhibition activity with an IC₅₀ of 40 nM but with slightly reduced selectivity over AKR1C2 (>2500 compared to >2800) (Table 1). A halogen scan at the para position revealed no correlation with electron-withdrawing effect and activity with a sequence of fluorine (A1-d)≤chlorine (A1-c)<bromine (A1-b) with IC₅₀'s of 60, 60 and 90 nM respectively. The same sequence correlation was apparent in the selectivity over AKR1C2, all of which were substantially increased compared with the reference compound. A positional sweep using fluorine revealed meta-fluoro A1-f was slightly more active than para-fluoro A1-d with both slightly more active than ortho-fluoro A1-e, with IC₅₀'s of 50, 60 and 70 nM respectively. Interestingly ortho-F A1-e possessed greater selectivity over AKR1C2 (>1430-fold), while meta-F A1-f possessed 960-fold selectivity. 2,4-Diflouro substitution (A1-g) resulted in reduced activity with an IC₅₀ of 130 nM. Substitution with the highly electronegative para-CF₃ moiety (A1-h) resulted in attenuated activity with an IC₅₀ of 200 nM.

Electron-donating substituents generally produced less potent AKR1C₃ inhibitors with para-methyl (A1-i), ethyl (A1-j) and isopropyl (A1-k) possessing values of 100 nM, 190 nM, and 190 nM respectively. The para-methoxy derivative (A1-l) possessed an IC₅₀ of 170 nM which was equipotent with para-cyano A1-m (IC₅₀=170 nM) while para-phenoxy (A1-n) afforded an IC₅₀ of 94 nM with just 51-fold selectivity over AKR1C2.

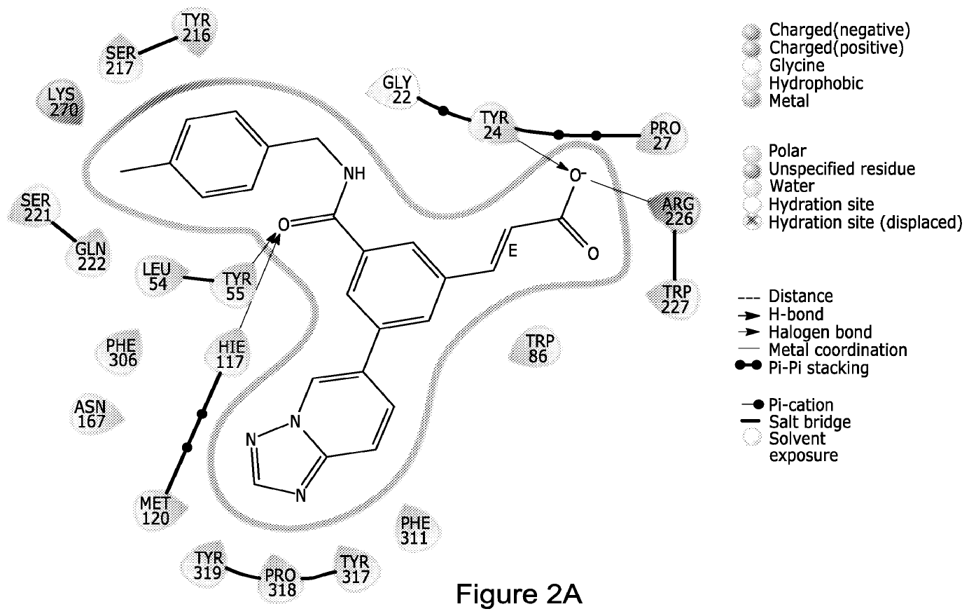

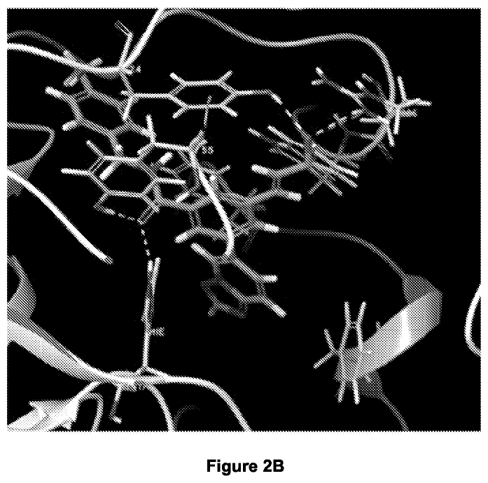

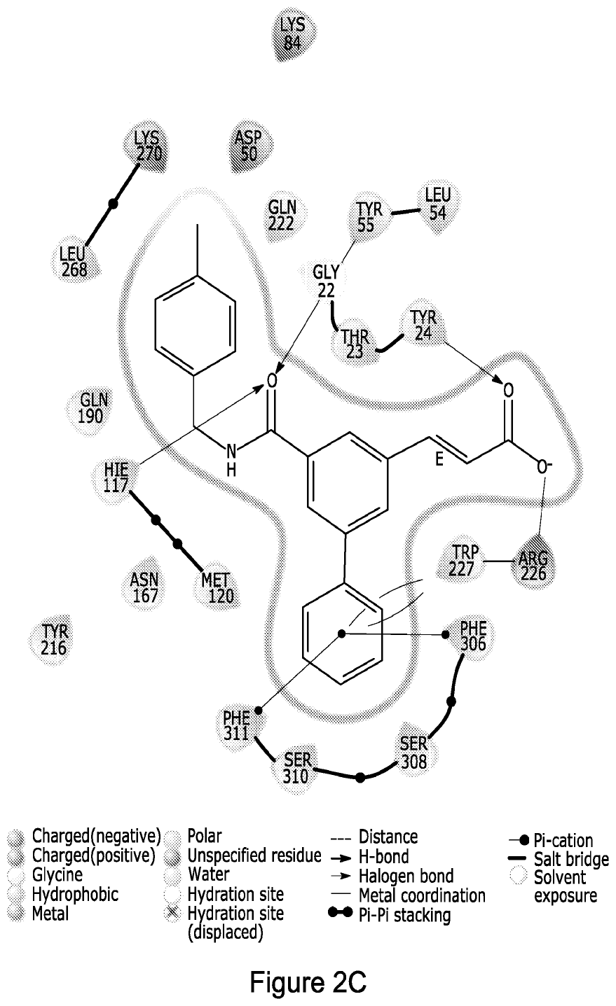

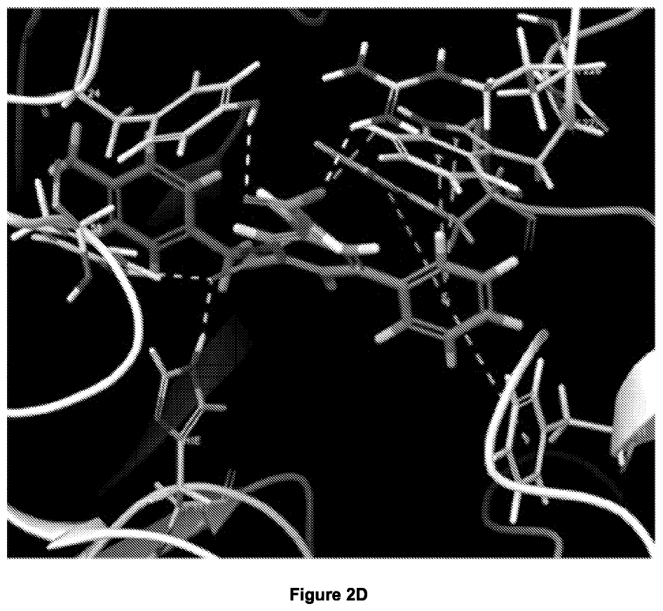

To further expand into the open pocket within the AKR1C3 steroid binding pocket (FIG. 2), a series of fused bicyclics were synthesized. Naphthyl A1-o possessed an IC₅₀ of 100 nM with 140-fold selectivity over AKR1C2. A nitrogen screen within the fused ring systems afforded an equipotent compound with quinoline A1-p while quinoxaline A1-q possessed slightly increased activity with an IC₅₀ of 94 nM. [1,2,4]triazolo[1,5-a]pyridine (A1-r) provided a compound with enhanced AKR1C3 inhibition over the reference compound, yielding an IC₅₀ of 51 nM and 1216-fold selectivity over AKR1C2. Addition of oxygens to the bicyclic ring in the form of 2,3-dihydrobenzo[b][1,4]dioxine A1-s afforded a compound with approximately two-fold less activity than A1-r with an IC₅₀ of 110 nM and reduced 200-fold selectivity over AKR1C2. Contraction of the ring to benzo[d][1,3]dioxole A1-t, afforded a compound with similar activity, IC₅₀ of 110 nM with 327-fold selectivity over AKR1C2.

Increasing length of the aromatic moiety with biphenyl A1-u restored activity with an IC₅₀ of 50 nM but with just 580-fold selectivity over AKR1C2. Substantially increasing the bulk of the aromatic moiety with 9-phenyl-9H-carbazole A1-v resulted in attenuation of activity with an IC₅₀ of 140 nM and just 221-fold selectivity over AKR1C2. Introduction of the ethynylbenzene ring A1-w achieved elongation and rotational constriction of the aromatic moiety and reduced activity and selectivity with an IC₅₀ of 180 nM and a much reduced 61-fold selectivity over AKR1C2. A cumene analogue A1-x, attenuated the activity against AKR1C3 (IC₅₀=880 nM) and exhibited 177-fold selectivity over AKR1C2. Addition of a 4-(2-fluorobenzyl) morpholine moiety (A1-y) considerably ameliorates activity and selectivity (IC₅₀=330 nM and 16-fold selectivity over AKR1C2).

Removal of one of the terminal methyl groups of the prenyl side chain affords (E)-but-2-ene A1-z, with an IC₅₀ of 110 nM and 354-fold selectivity over AKR1C2. Interestingly introduction of the geometric isomer (Z)-but-2-ene A1-aa afforded a compound with an IC₅₀ of 80 nM, similar to the prenyl side chain-containing reference compound, with 200-fold selectivity over AKR1C2, which is less than that seen with the reference compound. These data indicate that both terminal methyl groups are required for selective inhibition of AKR1C3. Introduction of ethynylcyclopropane A1-bb resulted in attenuated inhibition activity AKR1C3 (IC₅₀=500 nM) and selectivity (74-fold).

Prenyl ether 10 possessed improved activity to inhibit AKR1C3 (IC₅₀=80 nM) over the reference compound but attenuated selectivity over AKR1C2 (187-fold). This derivative is similar to the far more selective compound reported by Endo et. al. but with a metabolically stable retroamide bond, over a metabolically labile ester bond and had a different substitution pattern of the central phenyl ring. A simple bromine substituent at the prenyl position (11) ameliorates activity for AKR1C3, yielding an IC₅₀ of 300 nM.

Compounds of formula A-4 were next investigated. The results are shown in Table 3.

TABLE 3
AKR1C3 Inhibitory activity of compounds of formula (A4).
(A4)

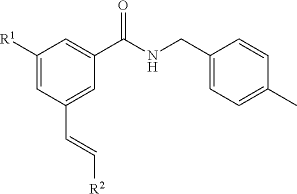

| Com- | | | IC₅₀ (μM) |
|---|---|---|---|
| pound | R¹ | R² | AKR1C3 |
| 3 | Br | CO₂CH₃ | 0.20 ± 0.020 |
| 12 | Br | CONH₂ | 28 ± 2.9 |
| 13 | 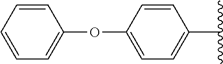 [SMILES: *c1ccc(Oc2ccccc2)cc1] | CONH₂ | 2.7 ± 0.37 |
| 14 | Br | CON(CH₃)₂ | 0.40 ± 0.06 |
| 15 | Br | CN | 0.17 ± 0.01 |
| 16a | 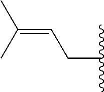 [SMILES: *CC=C(C)C] | CN | 1.4 ± 0.3 |
| 16b | 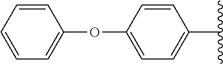 [SMILES: *c1ccc(Oc2ccccc2)cc1] | CN | 2.7 ± 0.37 |

It is known that esters and a boronic acid bioisostere suffer from completely ablated AKR1C3 inhibition activity. Surprisingly when bromo functionalized derivative 11 is protected as its methyl ester (3) its activity improves with IC₅₀ reducing from 300 nM to 200 nM. While still much less active than many other derivatives this led to the enticing possibility of identifying more active carboxylic acid derivatives.

Substitution of the carboxylic acid with a terminal amide (12) effectively inactivated the compound with IC₅₀ of 28 μM. This reduction in activity was confirmed with p-phenoxyphenyl derivative 13 possessing an IC₅₀ of 2.7 μM compared with the direct carboxylic acid bioisostere A1-n (IC₅₀=94 nM). Replacement of the carboxylic acid with a terminal N-dimethyl amide (14) increased activity >66-fold (IC₅₀=400 nM) over terminal primary amide 12. While a terminal cyano (15) retained some AKR1C3 inhibition activity with an IC₅₀ of 170 nM for the bromo-substituted derivative. However, this observation did not hold true for prenyl substituted terminal cyano compound 16a which possessed an IC₅₀ of 1.4 μM compared with its carboxylic acid counterpart (IC₅₀=70 nM; Table 1 reference compound) nor p-phenoxylphenyl substituted terminal cyano compound 16b which exhibited an IC₅₀ of 2.7 μM compared with its carboxylic acid counterpart A1-n (IC₅₀=94 nM).

Compounds of formula (A-3) were next investigated. The results are shown in Table 4.

TABLE 4
Inhibitory activity and selectivity of compounds of formula (A3).
(A3)

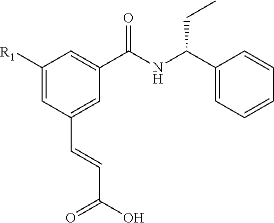

| | | IC₅₀ (μM) | | | | Selectivity |
|---|---|---|---|---|---|---|
| Cpd | R₁ | AKR1C3 | AKR1C1 | AKR1C2 | AKR1C4 | 1C2:1C3 |
| 20a | 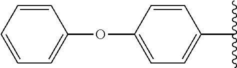 [SMILES: *c1ccc(Oc2ccccc2)cc1] | 0.12 ± 0.014 | N.D. | 6.0 ± 1.4 | N.D. | 51 |
| 20b | 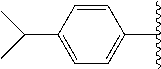 [SMILES: *c1ccc(C(C)C)cc1] | 0.26 ± 0.0166 | N.D. | N.D. | N.D. | N.D. |

The p-phenoxylphenyl substituted compound 20a possessed attenuated activity (IC₅₀=120 nM) over its non-benzylic substituted counterpart A1-n (IC₅₀=94 nM) but identical selectivity over AKR1C2. Modification to the benzylic position of p-isopropyl phenyl (20b) similarly reduced activity (IC₅₀=260 nM) compared with its non-benzylic substituted counterpart A1-k (IC₅₀=190 nM). Without wishing to be bound to any particular theory, It is possible that the ethyl moiety is hindering crucial hydrogen bond formation between the amide carbonyl and AKR1C₃ binding site amino acid residues.

Computation Modelling Study: In silico docking studies were employed to understand predicted binding interactions between inhibitors (A1-r and A1-a) and the AKR1C3 protein (PDB ID: 3UG8, AKR1C3.NADP+indomethacin) using Schrodinger-2022-3 software (FIG. 2). These studies predict key hydrogen bonding interactions between the amide carbonyl oxygen (A1-r) and TYR55 and HIS117 residues. This carbonyl group directly attached to the central phenyl ring forms stronger hydrogen bonds within the oxyanion site and shorter hydrogen bond distance with the amino acid residues, which results in superior selectivity and inhibitory potency as we have previously reported. Most AKR1C3 ligands anchor to the oxyanion site via the presence of either a carbonyl or carboxylate groups forming strong hydrogen bonds with TYR55 and HIS117, bringing the ligands in close proximity to the nicotinamide head group. The carboxylic acid side chain is predicted to bind in the sub-pocket 3 (SP3) region forming hydrogen bonding interactions with TYR24 and a salt bridge with ARG226 (FIGS. 2A & B). The [1,2,4]triazolo[1,5-a]pyridin-6-yl moiety of A1-r is predicted to occupy the hydrophobic region sub-pocket 1 (SP1) and surrounding residues (PHE311, TYR317, PRO318, and TRY319) forming strong hydrophobic interactions and potentially accounting for selectivity over the closely related AKR1C2 isoform.

The phenyl derivative's (A1-a) amide carbonyl group is predicted to engage in the same hydrogen bond interactions within the oxyanion site of AKR1C3 and the carboxylate ion is predicted to form hydrogen bond interactions with TYR24 and a salt bridge with the ARG226 residue. Additionally, this compound's phenyl group has predicted very strong hydrophobic interactions with TRP227, PHE306 and PHE311 in the SP2 region, which contributes to greater selectivity (>2320 fold) for AKR1C3 over AKR1C2 (FIGS. 2C & D).

In Vitro Plasma and Microsome Stability: Compound A1-r, possessing the best combination of activity, selectivity and favorable metabolic structure (i.e., no exposed phenyl ring) was evaluated to determine plasma stability, including a prodrug strategy to overcome the inherent absorption, distribution, metabolism and excretion (ADME) limitations often encountered with free carboxylic acid drugs. Thus, methyl ester intermediate 4r as the prodrug of choice.

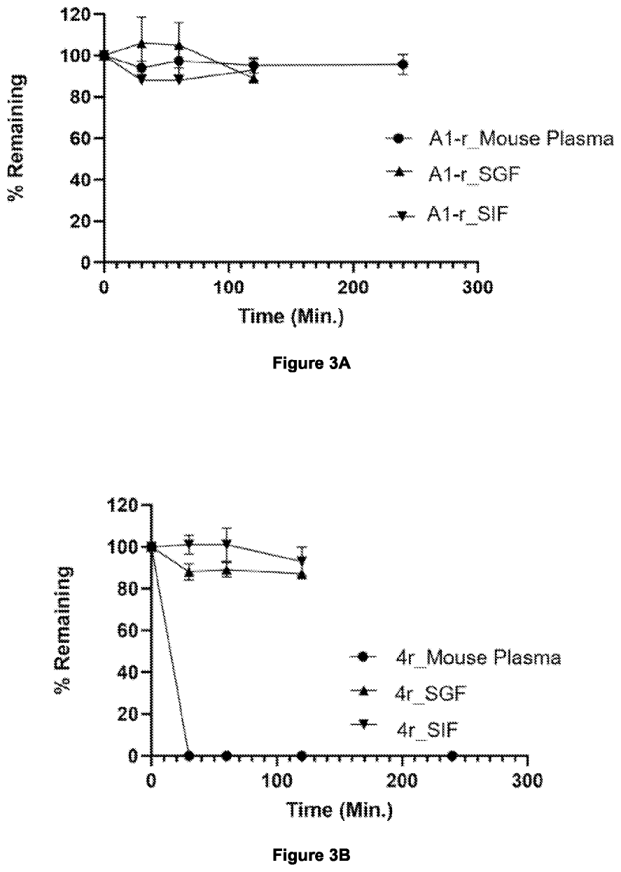

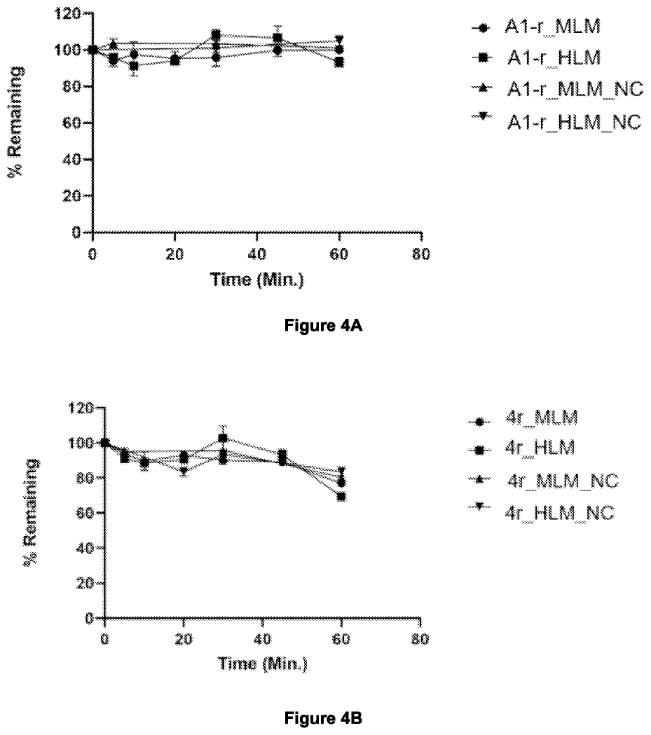

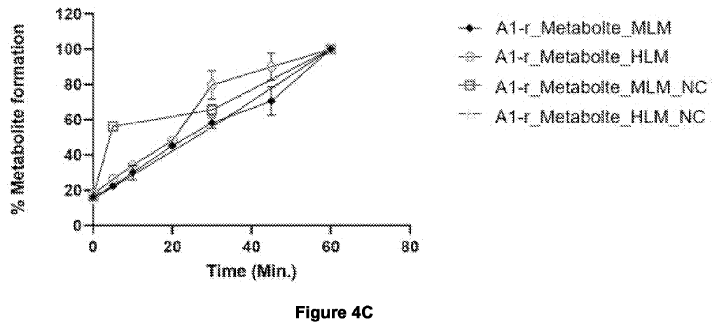

Gastrointestinal (GI) fluid stability studies were performed in simulated gastric fluid (SGF) and simulated intestinal fluid (SIF). The 4r and A1-r were stable in SGF for 1 h and SIF for 2 h, suggested that the these compounds were favorable for oral administration (FIG. 3a & b). Moreover, the methyl ester 4r was unstable in mouse plasma, being rapidly converted into A1-r. The ester was reduced to below detectable limits within 30 minutes of exposure to mouse plasma while the free acid A1-r was stable for over 240 minutes (FIG. 3 a, b). In vitro metabolic stability of 4r and A1-r were investigated using mouse (MLM) and human liver microsomes (HLM). The result of the metabolic stability study was expressed as the % parent remaining and formation of free acid A1-r different time points relative to the parent at 0 minutes (100% parent) (FIG. 4 a, b, c). The free acid A1-r was stable in MLM, HLM and negative control (NC), indicating no or limited non-CYP metabolism (FIG. 4a). Compound 4r was moderately degraded (approximately 25%) in MLM, HLM, as well as NC, indicating non-CYP-mediated metabolism (FIG. 4b). Ester 4r was found to be hydrolyzed to free acid A1-r by both MLM, HLM and NC at a similar rate over 60 minutes (FIG. 4c). Thus, the methyl ester 4r possessed good potential for use as a prodrug AKR1C3 inhibitor in both mouse and human.

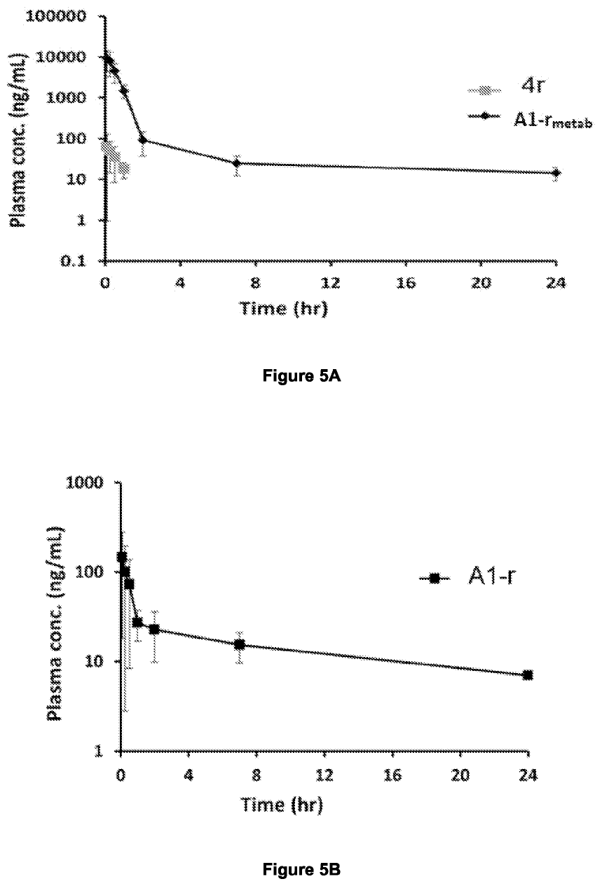

In Vivo Pharmacokinetics: Given the favourable activity, selectivity, in vitro stability and release data of compounds 4r and A1-r, a pharmacokinetic study was performed (Table 4). The plasma concentration vs. time profile for 4r and the metabolite (A1-rₘₑₜₐ) is shown in FIGS. 5a & b following oral administration (10 mg/Kg) of 4r or 4r in male Balb/C mice (mean±SD, n=5). The absorption of A1-r from the gastrointestinal tract was rapid as it could be detected in plasma within 5 minutes and A1-r_{metab} was rapidly formed from the methyl ester prodrug 4r. The prodrug 4r was undetectable in systemic circulation 1 hour post administration but A1-r_{metab} was detected at the last study point (24 hours) (FIG. 5a).

Pharmacokinetic parameters for 4r and 5r are shown in Table 5. Following administration of 4r, the metabolite A1-r_{metab} has significantly greater exposure (AUCₒ₋ₜ and AUC_{o-INF} 17-fold and 13-fold higher respectively) than direct administration of A1-r and an increase in peak plasma concentration (Cₘₐₓ>66-fold higher than following A1-r administration). Prodrug 4r was rapidly converted to A1-r_{metab} and resulted in greater drug exposure following an equivalent oral dose of A1-r. These parameters validate our prodrug approach with the released active AKR1C3 inhibitor metabolite A1-r_{metab} possessing improved pharmacokinetic characteristics compared to the directly administered compound 5 A1-r.

TABLE 5
Pharmacokinetic parameters of compounds 4r, A1-r_{metabolite} and A1-r after oral
administration (10 mg/kg) of 4r and A1-r in Balb/c mice (mean ± SD, n = 5).

| | t_{1/2} | Tₘₐₓ | Cₘₐₓ | AUC₀₋ₜ | AUC_{0-INF} | Extrapolated | V_{z/F} | CI/F |
|---|---|---|---|---|---|---|---|---|
| Cpd | (h) | (h) | (ng/mL) | (h*ng/mL) | (h*ng/mL) | AUC % | (L/Kg) | (L/h/kg) |
| 4r | N/A¹ | 0.317 ± | 92.796 ± | 36.416 ± | N/A | N/A | N/A | N/A |
| | | 0.181 | 52.081 | 10.366 | | | | |
| A1- | 8.9 ± | 0.2 ± | 11599.4 ± | 6382.8 ± | 6550.1 ± | N/A | N/A | N/A |
| r_{metab} | 4.4 | 0.2 | 5262.7 | 1023.2 | 1031.9 | | | |
| A1-r | 14.4 ± | 0.1 ± | 174.8 ± | 367.6 ± | 515.1 ± | 28.8 ± | 405.0 ± | 19.4 ± |
| | 4.2 | 0.1 | 138.5 | 57.7 | 23.7 | 10.0 | 119.8 | 0.9 |

¹Not applicable.

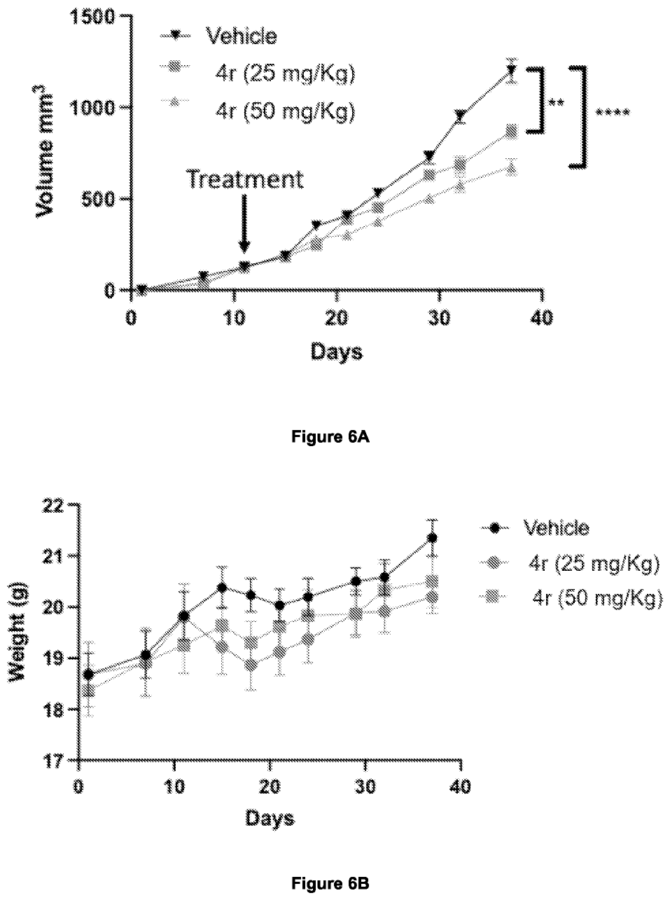

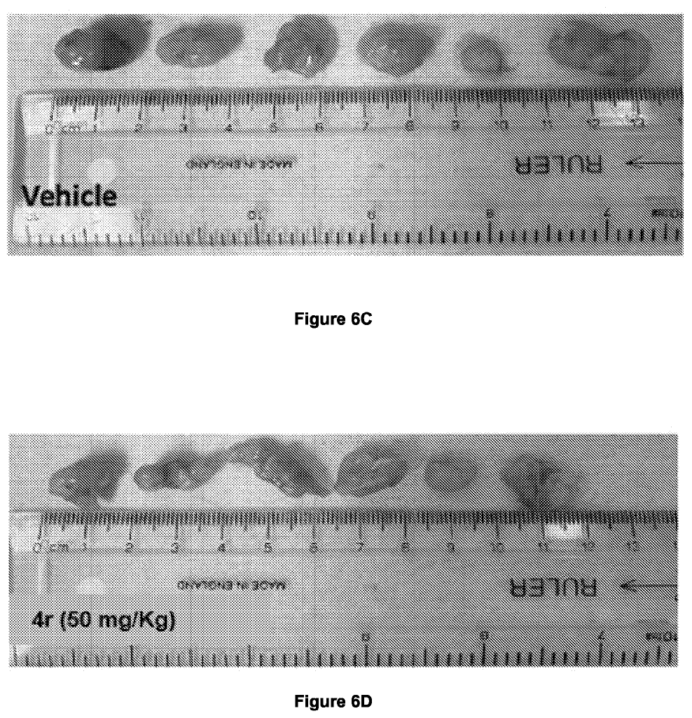

In Vivo Antitumor Activity: The efficacy of compound 4r was next evaluated in vivo in a 22Rv1 tumor xenograft model of prostate cancer which is reported to be resistant to the clinical gold standard Enzalutamide.⁴ Five week old NSG mice were implanted with 22Rv1 cells and treatment with 4r was initiated on day 11 when tumors reached a mean volume of 125 mm³. Doses of 25 mg/Kg and 50 mg/Kg of 4r were administered once a day intraperitoneally (IP) for a total of 26 days (FIG. 6). A clear dose-dependent relationship was observed with a 25 mg/Kg dose of 4r significantly reducing tumor volume by approximately 30%. Gratifyingly, a 50 mg/Kg dose of 4r significantly reduced tumor burden by approximately 45% (FIG. 6A). This in vivo efficacy confirms effective conversion of the inactive prodrug 4r into the active AKR1C3 inhibitor A1-r in mice. Furthermore, reduction of tumor burden was combined with no observed loss of mouse body weight at either dose, indeed the mice continued to grow normally (FIG. 6B). Excised tumors supported a reduction of tumor mass (FIGS. 6C &D).

The disclosure provides compounds of formula (A) which possess superior activity and/or selectivity via rational drug design and detailed structure-activity relationship studies. Our efforts identified A1-r possessing a [1,2,4]triazolo[1,5-a]pyridine moiety that exploits the open pocket present in AKR1C3, but absent in 1C1 and 1C2, to afford a highly active and selective AKR1C3 inhibitor. Wishing to subvert potential pharmacokinetic issues inherent with carboxylic acid-based drugs we identified inactive methyl ester 4r as a suitable prodrug which rapidly released the active AKR1C3 inhibitor A1-r in both human and mouse liver microsomes. The prodrug achieved 17-fold greater exposure and far greater peak serum concentration than direct administration of A1-r in in vivo pharmacokinetic studies. The prodrug demonstrated dose-dependent reduction of tumor volume in a 22Rv1 prostate cancer xenograft model. Taken together this study identifies both new AKR1C3 inhibitors for further development and suggests the general scaffold is suitable for prodrug design to optimize new compounds for the treatment of prostate and other cancers.

### REFERENCES

Winter, S. S.; Dunsmore, K. P; Devidas, M.; Wood, B. L.; Esiashvili, N.; Chen, Z.; Eisenberg, N.; Briegel, N.; Hayashi, R. J.; Gastier-Foster, J. M.; Carroll, A. J.; Heerema, N. A.; Asselin, B. L.; Gaynon, P. S.; Borowitz, M. J.; Loh, M. L.; Rabin, K. R.; Raetz, E. A.; Zweidler-Mckay, P. A.; Winick, N. J.; Carroll, W. L.; Hunger, S. P. Improved Survival for Children and Young Adults With T-Lineage Acute Lymphoblastic Leukemia: Results From the Children's Oncology Group AALL0434 Methotrexate Randomization. J. Clin. Oncol. 2018, 36, 2926-2934.
Jemal, A.; Siegel, R.; Ward, E; Hao, Y.; Xu, J.; Murray, T.; Thun, M. J. Cancer statistics. CA Cancer J Clin. 2008, 58, 71-96.
Pui, C. H.; Evans, W. E.; Treatment of acute lymphoblastic leukemia. N. Engl. J. Med. 2006, 354, 166-78.
Graux, C. Biology of acute lymphoblastic leukemia (ALL): clinical and therapeutic relevance. Transfus Apheresis Sci. 2011, 44, 183-189.
Smith, M. A.; Altekruse, S. F.; Adamson, P. C.; Reaman, G. H.; Seibel, N. L. Declining childhood and adolescent cancer mortality. Cancer. 2014, 120, 2497-506.
Jeha, S.; Pei, D.; Choi, J.; Cheng, C.; Sandlund, J. T.; Coustan-Smith, E.; Campana, D.; Inaba, H.; Rubnitz, J. E.; Ribeiro, R. C.; Gruber, T. A.; Raimondi, S. C.; Khan, R. B.; Yang, J. J.; Mullighan, C. G.; Downing, J. R.; Evans, W, E.; Relling, M. V.; Pui, C. H. Improved CNS Control of Childhood Acute Lymphoblastic Leukemia Without Cranial Irradiation: St Jude Total Therapy Study 16. J. Clin. Oncol. 2019, 37, 3377-3391.
Goldberg, J. M.; Silverman, L. B; Levy, D. E.; Dalton, V. K.; Gelber, R. D.; Lehmann, L.; Cohen, H. J.; Sallan, S. E.; Asselin, B. L. Childhood T-cell acute lymphoblastic leukemia: the Dana-Farber Cancer Institute acute lymphoblastic leukemia consortium experience. J. Clin. Oncol. 2003, 3616-3622.
Van Vlierberghe, P.; Ferrando, A. The molecular basis of T cell acute lymphoblastic leukemia. J. Clin. Invest. 2012, 122, 3398-406.
Penning, T. M.; Drury, J. E. Human aldo-keto reductases: Function, gene regulation, and single nucleotide polymorphisms. Arch. Biochem. Biophys. 2007, 464, 241-250.
Reddi, D.; Seaton, B. W.; Woolston, D.; Aicher, L.; Monroe, L. D.; Mao, Z. J.; Harrell, J. C.; Radich, J. P.; Advani, A.; Papadantonakis, N.; Yeung, C. C. S. AKR1C3 expression in T acute lymphoblastic leukemia/lymphoma for clinical use as a biomarker. Sci Rep. 2022, 12, 5809. (PMID: 35388063)
Penning, T. M.; Burczynski, M. E.; Jez, J. M.; Hung, C. F.; Lin, H. K.; Ma, H.; Moore, M.; Palackal, N.; Ratnam, K. Human 3alpha-hydroxysteroid dehydrogenase isoforms (AKR1C1-AKR1C4) of the aldo-keto reductase superfamily: functional plasticity and tissue distribution reveals roles in the inactivation and formation of male and female sex hormones. Biochem. J. 2000, 351, 67-77.
Chen, W. D.; Zhang, Y. Regulation of aldo-keto reductases in human diseases. Front. Pharmacol. 2012, 3, 35.
Penning, T. M.; Byrns, M. C. Steroid hormone transforming aldo-keto reductases and cancer. Ann. N. Y. Acad. Sci. 2009, 1155, 33-42.
Fung, K. M.; Samara, E. N.; Wong, C.; Metwalli, A.; Krlin, R.; Bane, B.; Liu, C. Z.; Yang, J. T.; Pitha, J. V.; Culkin, D. J.; Kropp, B. P.; Penning, T. M.; Lin, H. K. Increased expression of type 2 3alpha-hydroxysteroid dehydrogenase/type 5 17beta-hydroxysteroid dehydrogenase (AKR1C3) and its relationship with androgen receptor in prostate carcinoma. Endocr. Relat. Cancer. 2006, 13, 169-180.
Stanbrough, M.; Bubley, G. J.; Ross, K.; Golub, T. R.; Rubin, M. A.; Penning, T. M.; Febbo, P. G.; Balk, S. P. Increased expression of genes converting adrenal androgens to testosterone in androgen-independent prostate cancer. Cancer Res., 2006, 66, 2815-2825.
Komoto, J.; Yamada, T.; Watanabe, K.; Takusagawa, F. Crystal structure of human prostaglandin F synthase (AKR1C3). Biochemistry 2004, 43, 2188-2198.
Penning, T. M. The aldo-keto reductases (AKRs): Overview. Chem Biol Interact. 2015 234, 236-246. PMID: 25304492.
Penning, T. M.; Wangtrakuldee, P.; Auchus, R. J. Structural and Functional Biology of Aldo-Keto Reductase Steroid-Transforming Enzymes. Endocr Rev. 2019, 40, 447-475. PMID: 30137266
Steckelbroeck, S.; Jin, Y.; Gopishetty, S.; Oyesanmi, B.; Penning, T. M. Human cytosolic 3α-hydroxysteroid dehydrogenases of the aldo-keto reductase superfamily display significant 3β-hydroxysteroid dehydrogenase activity: implications for steroid hormone metabolism and action. J. Biol. Chem. 2003, 279, 10784-10795.
Ji, Q.; Chang, L.; Stanczyk, F. Z.; Ookhtens, M.; Sherrod, A.; Stolz, A. Impaired dihydrotestosterone catabolismin human prostate cancer: critical role of AKR1C2 as a pre-receptor regulator of androgen receptor signaling. Cancer Res. 2007, 67, 1361-1369.
Penning, T. M.; Bauman, D. R.; Jin, Y.; Rizner, T. L. Identification of the molecular switch that regulates access of 5α-DHT to the androgen receptor. Mol. Cell. Endocrinol. 2007, 265-266, 77-82.
Hofman, J.; Malcekova, B.; Skarka, A.; Novotna, E.; Wsol, V. Anthracycline resistance mediated by reductive metabolism in cancer cells: The role of aldo-keto reductase 1C3. Toxicol. Appl. Pharmacol. 2014, 278, 238-248.
Heibein, A. D.; Guo, B.; Sprowl, J. A.; MacLean, D. A.; Parissenti, A. M. Role of aldo-keto reductases and other doxorubicin pharmacokinetic genes in doxorubicin resistance, DNA binding, and subcellular localization. BMC Cancer 2012, 12, 381.
Rižner, T. L.; Penning, T. M. Role of aldo-keto reductase family 1 (AKR1) enzymes in human steroid metabolism. Steroids, 2014, 79, 49-63. PMID: 24189185
Penning, T. M. Aldo-Keto Reductase (AKR) 1C3 inhibitors: a patent review. Expert Opin Ther Pat. 2017, 27, 1329-1340. PMID: 28895472
Byrns, M. C.; Jin, Y.; Penning, T. M. Inhibitors of type 5 17β-hydroxysteroid dehydrogenase (AKR1C3): overview and structural insights. J. Steroid Biochem. Mol. Biol. 2011, 125, 95-104. PMID: 21087665
Liu, Y.; He, S.; Chen, Y.; Liu, Y.; Feng, F.; Liu, W.; Guo, Q.; Zhao, L.; Sun, H. Overview of AKR1C3: Inhibitor Achievements and Disease Insights. J Med Chem. 2020, 63, 11305-11329. PMID: 32463235.
Bauman, D. R.; Rudnick, S. I.; Szewczuk, L. M.; Jin, Y.; Gopishetty, S.; Penning, T. M. Development of nonsteroidal anti-inflammatory drug analogs and steroid carboxylates selective for human aldo-keto reductase isoforms: potential antineoplastic agents that work independently of cyclooxygenase isozymes. Mol. Pharmacol. 2005, 67, 60-68. PMID: 15475569.
Adeniji, A.; Uddin, M. J.; Zang, T.; Tamae, D.; Wangtrakuldee, P.; Marnett, L. J.; Penning, T. M. Discovery of ®-2-(6-Methoxynaphthalen-2-yl) butanoic Acid as a Potent and Selective Aldo-keto Reductase 1C3 Inhibitor. J Med Chem. 2016, 59, 7431-7444. PMID: 27486833.
Hara, A.; Endo, S.; Matsunaga, T.; Soda, M.; Yashiro, K.; El-Kabbani, O. Long-chain fatty acids inhibit human members of the aldo-keto reductase 1C subfamily. J Biochem. 2017, 162, 371-379. PMID: 28992312.
Endo, S.; Matsunaga, T.; Kanamori, A.; Otsuji, Y.; Nagai, H.; Sundaram, K.; El-Kabbani, O.; Toyooka, N.; Ohta, S.; Hara, A. Selective inhibition of human type-5 17β-hydroxysteroid dehydrogenase (AKR1C3) by baccharin, a component of Brazilian propolis. J Nat Prod. 2012, 75, 716-721. PMID: 22506594.
Verma, K.; Gupta, N.; Zang, T.; Wangtrakluldee, P.; Srivastava, S. K.; Penning, T. M.; Trippier, P. C. AKR1C3 Inhibitor KV-37 Exhibits Antineoplastic Effects and Potentiates Enzalutamide in Combination Therapy in Prostate Adenocarcinoma Cells. Mol Cancer Ther. 2018, 17, 1833-1845. PMID: 29891491.
Verma, K.; Zang, T.; Penning, T. M.; Trippier, P. C. Potent and Highly Selective Aldo-Keto Reductase 1C3 (AKR1C3) Inhibitors Act as Chemotherapeutic Potentiators in Acute Myeloid Leukemia and T-Cell Acute Lymphoblastic Leukemia. J Med Chem. 2019, 62, 3590-3616. PMID: 30836001
Zang, T.; Verma, K.; Chen, M.; Jin, Y.; Trippier, P. C.; Penning, T. M. Screening baccharin analogs as selective inhibitors against type 5 17β-hydroxysteroid dehydrogenase (AKR1C3). Chem Biol Interact. 2015, 234, 339-48. PMID: 25555457.
Verma, K.; Zang, T.; Gupta, N.; Penning, T. M.; Trippier, P. C. Selective AKR1C3 Inhibitors Potentiate Chemotherapeutic Activity in Multiple Acute Myeloid Leukemia (AML) Cell Lines. ACS Med Chem Lett. 2016, 7, 774-779. PMID: 27563402.
Endo, S.; Hu, D.; Matsunaga, T.; Otsuji, Y.; El-Kabbani, O.; Kandeel, M.; Ikari, A.; Hara, A.; Kitade, Y.; Toyooka, N. Synthesis of non-prenyl analogues of baccharin as selective and potent inhibitors for aldo-keto reductase 1C3. Bioorg Med Chem. 2014, 22, 5220-5233. PMID: 25182963.
Liedtke, A. J.; Adeniji, A. O.; Chen, M.; Byrns, M. C.; Jin, Y.; Christianson, D. W.; Marnett, L. J.; Penning, T. M. Development of potent and selective indomethacin analogues for the inhibition of AKR1C3 (Type 5 17β-hydroxysteroid dehydrogenase/prostaglandin F synthase) in castrate-resistant prostate cancer. J Med Chem. 2013, 56: 2429-2446. PMID: 23432095.
Lolli, M. L.; Carnovale, I. M.; Pippione, A. C.; Wahlgren, W. Y.; Bonanni, D.; Marini, E.; Zonari, D.; Gallicchio, M.; Boscaro, V.; Goyal, P.; Friemann, R.; Rolando, B.; Bagnati, R.; Adinolfi, S.; Oliaro-Bosso, S.; Boschi, D. Bioisosteres of Indomethacin as Inhibitors of Aldo-Keto Reductase 1C3. ACS Med Chem Lett. 2019, 10, 437-443. PMID: 30996776.
Pippione, A. C.; Carnovale, I. M.; Bonanni, D.; Sini, M.; Goyal, P.; Marini, E.; Pors, K.; Adinolfi, S.; Zonari, D.; Festuccia, C.; Wahlgren, W. Y.; Friemann, R.; Bagnati, R.; Boschi, D.; Oliaro-Bosso, S.; Lolli, M. L. Potent and selective aldo-keto reductase 1C3 (AKR1C3) inhibitors based on the benzoisoxazole moiety: application of a bioisosteric scaffold hopping approach to flufenamic acid. Eur J Med Chem. 2018, 150, 930-945. PMID: 29602039.
Endo, S.; Oguri, H.; Segawa, J.; Kawai, M.; Hu, D.; Xia, S.; Okada, T.; Irie, K.; Fujii, S.; Gouda, H.; Iguchi, K.; Matsukawa, T.; Fujimoto, N.; Nakayama, T.; Toyooka, N.; Matsunaga, T.; Ikari, A. Development of Novel AKR1C3 Inhibitors as New Potential Treatment for Castration-Resistant Prostate Cancer. J Med Chem. 2020, 63, 10396-10411. PMID: 32847363.
Yin, Y. D.; Fu, M.; Brooke, D. G.; Heinrich, D. M.; Denny, W. A.; Jamieson, S. M. The Activity of SN33638, an Inhibitor of AKR1C3, on Testosterone and 17β-Estradiol Production and Function in Castration-Resistant Prostate Cancer and ER-Positive Breast Cancer. Front Oncol. 2014, 8, 159. PMID: 24995161
Loriot, Y.; Fizazi, K.; Jones, R. J.; Van den Brande, J.; Molife, R. L.; Omlin, A.; James, N. D.; Baskin-Bey, E.; Heeringa, M.; Baron, B.; Holtkamp, G. M.; Ouatas, T.; De Bono, J. S. Safety, tolerability and anti-tumour activity of the androgen biosynthesis inhibitor ASP9521 in patients with metastatic castration-resistant prostate cancer: multi-centre phase I/II study. Invest New Drugs. 2014, 32, 995-1004. PMID: 24771350.
Kikuchi, A.; Furutani, T.; Azami, H.; Watanabe, K.; Niimi, T.; Kamiyama, Y.; Kuromitsu, S.; Baskin-Bey, E.; Heeringa, M.; Ouatas, T.; Enjo, K. In vitro and in vivo characterisation of ASP9521: a novel, selective, orally bioavailable inhibitor of 17β-hydroxysteroid dehydrogenase type 5 (17BHSD5; AKR1C3). Invest New Drugs. 2014, 32, 860-870. PMID: 24981575.
Rižner, T. L.; Penning, T. M. Aldo-keto reductase 1C3-Assessment as a new target for the treatment of endometriosis. Pharmacol Res. 2020, 152, 104446. PMID: 31546014.
Moradi Manesh, D.; El-Hoss, J.; Evans, K.; Richmond, J.; Toscan, C. E.; Bracken, L. S.; Hedrick, A.; Sutton, R.; Marshall, G. M.; Wilson, W. R.; Kurmasheva, R. T.; Billups, C.; Houghton, P. J.; Smith, M. A.; Carol, H.; Lock, R. B. AKR1C3 is a biomarker of sensitivity to PR-104 in preclinical models of T-cell acute lymphoblastic leukemia. Blood. 2015, 126, 1193-1202. PMID: 26116659.
Brawley, O. W. Prostate cancer epidemiology in the United States. World J Urol 2012, 30, 195-200.
Miyamoto, H.; Messing, E. M.; Chang, C. Androgen deprivation therapy for prostate cancer: current status and future prospects. Prostate 2004, 61, 332-353.
Kirby, M.; Hirst, C.; Crawford, E. D. Characterising the castration-resistant prostate cancer population: a systematic review. Int J Clin Pract 2011, 65, 1180-1192.
Wadosky, K. M.; Koochekpour, S. Androgen receptor splice variants and prostate cancer: From bench to bedside. Oncotarget 2017, 8, 18550-18576.
Locke, J. A.; Guns, E. S.; Lubik, A. A.; Adomat, H. H.; Hendy, S. C.; Wood, C. A.; Ettinger, S. L.; Gleave, M. E.; Nelson, C. C. Androgen levels increase by intratumoral de novo steroidogenesis during progression of castration-resistant prostate cancer. Cancer Res 2008, 68, 6407-6415.
Penning, T. M. Androgen biosynthesis in castration-resistant prostate cancer. Endocr Relat Cancer 2014, 21, T67-78.
Buttigliero, C.; Tucci, M.; Bertaglia, V.; Vignani, F.; Bironzo, P.; Di Maio, M.; Scagliotti, G. V. Understanding and overcoming the mechanisms of primary and acquired resistance to abiraterone and enzalutamide in castration resistant prostate cancer. Cancer Treat Rev 2015, 41, 884-892.
Scher, H. I.; Fizazi, K.; Saad, F.; Taplin, M. E.; Sternberg, C. N.; Miller, K.; de Wit, R.; Mulders, P.; Chi, K. N.; Shore, N. D.; Armstrong, A. J.; Flaig, T. W.; Flechon, A.; Mainwaring, P.; Fleming, M.; Hainsworth, J. D.; Hirmand, M.; Selby, B.; Seely, L.; de Bono, J. S.; Investigators, A. Increased survival with enzalutamide in prostate cancer after chemotherapy. N Engl J Med 2012, 367, 1187-1197.
Fujimoto, N. Novel agents for castration-resistant prostate cancer: Early experience and beyond. Int J Urol 2016, 23, 114-121.
Morsy, A.; Trippier, P. C. Reversal of Apalutamide and Darolutamide Aldo-Keto Reductase 1C3-Mediated Resistance by a Small Molecule Inhibitor. ACS Chem Biol 2020, 15, 646-650.
Penning, T. M.; Burczynski, M. E.; Jez, J. M.; Hung, C. F.; Lin, H. K.; Ma, H.; Moore, M.; Palackal, N.; Ratnam, K. Human 3alpha-hydroxysteroid dehydrogenase isoforms (AKR1C1-AKR1C4) of the aldo-keto reductase superfamily: functional plasticity and tissue distribution reveals roles in the inactivation and formation of male and female sex hormones. Biochem J 2000, 351, 67-77.
Penning, T. M.; Drury, J. E. Human aldo-keto reductases: Function, gene regulation, and single nucleotide polymorphisms. Arch Biochem Biophys 2007, 464, 241-250.
Penning, T. M.; Byrns, M. C. Steroid hormone transforming aldo-keto reductases and cancer. Ann N Y Acad Sci 2009, 1155, 33-42.
Fung, K. M.; Samara, E. N.; Wong, C.; Metwalli, A.; Krlin, R.; Bane, B.; Liu, C. Z.; Yang, J. T.; Pitha, J. V.; Culkin, D. J.; Kropp, B. P.; Penning, T. M.; Lin, H. K. Increased expression of type 2 3alpha-hydroxysteroid dehydrogenase/type 5 17beta-hydroxysteroid dehydrogenase (AKR1C3) and its relationship with androgen receptor in prostate carcinoma. Endocr Relat Cancer 2006, 13, 169-180.
Penning, T. M. The aldo-keto reductases (AKRs): Overview. Chem Biol Interact 2015, 234, 236-246.
Penning, T. M.; Wangtrakuldee, P.; Auchus, R. J. Structural and Functional Biology of Aldo-Keto Reductase Steroid-Transforming Enzymes. Endocr Rev 2019, 40, 447-475.
Steckelbroeck, S.; Jin, Y.; Gopishetty, S.; Oyesanmi, B.; Penning, T. M. Human cytosolic 3alpha-hydroxysteroid dehydrogenases of the aldo-keto reductase superfamily display significant 3beta-hydroxysteroid dehydrogenase activity: implications for steroid hormone metabolism and action. J Biol Chem 2004, 279, 10784-10795.
Ji, Q.; Chang, L.; Stanczyk, F. Z.; Ookhtens, M.; Sherrod, A.; Stolz, A. Impaired dihydrotestosterone catabolismin human prostate cancer: critical role of AKR1C2 as a pre-receptor regulator of androgen receptor signaling. Cancer Res 2007, 67, 1361-1369.
Penning, T. M.; Bauman, D. R.; Jin, Y.; Rizner, T. L. Identification of the molecular switch that regulates access of 5alpha-DHT to the androgen receptor. Mol Cell Endocrinol 2007, 265-266, 77-82.
Byrns, M. C.; Jin, Y.; Penning, T. M. Inhibitors of type 5 17beta-hydroxysteroid dehydrogenase (AKR1C3): overview and structural insights. J Steroid Biochem Mol Biol 2011, 125, 95-104.
Hofland, J.; van Weerden, W. M.; Dits, N. F.; Steenbergen, J.; van Leenders, G. J.; Jenster, G.; Schroder, F. H.; de Jong, F. H. Evidence of limited contributions for intratumoral steroidogenesis in prostate cancer. Cancer Res 2010, 70, 1256-1264.
Hamid, A. R.; Pfeiffer, M. J.; Verhaegh, G. W.; Schaafsma, E.; Brandt, A.; Sweep, F. C.; Sedelaar, J. P.; Schalken, J. A. Aldo-keto reductase family 1 member C3 (AKR1C3) is a biomarker and therapeutic target for castration-resistant prostate cancer. Mol Med 2013, 18, 1449-1455.
Penning, T. M.; Jonnalagadda, S.; Trippier, P. C.; Rizner, T. L. Aldo-Keto Reductases and Cancer Drug Resistance. Pharmacol Rev 2021, 73, 1150-1171.
Liu, C.; Armstrong, C. M.; Lou, W.; Lombard, A.; Evans, C. P.; Gao, A. C. Inhibition of AKR1C3 Activation Overcomes Resistance to Abiraterone in Advanced Prostate Cancer. Mol Cancer Ther 2017, 16, 35-44.
Liu, C.; Lou, W.; Zhu, Y.; Yang, J. C.; Nadiminty, N.; Gaikwad, N. W.; Evans, C. P.; Gao, A. C. Intracrine Androgens and AKR1C3 Activation Confer Resistance to Enzalutamide in Prostate Cancer. Cancer Res 2015, 75, 1413-1422.
Liu, C.; Yang, J. C.; Armstrong, C. M.; Lou, W.; Liu, L.; Qiu, X.; Zou, B.; Lombard, A. P.; D'Abronzo, L. S.; Evans, C. P.; Gao, A. C. AKR1C3 Promotes AR-V7 Protein Stabilization and Confers Resistance to AR-Targeted Therapies in Advanced Prostate Cancer. Mol Cancer Ther 2019, 18, 1875-1886.
Zheng, Z.; Li, J.; Liu, Y.; Shi, Z.; Xuan, Z.; Yang, K.; Xu, C.; Bai, Y.; Fu, M.; Xiao, Q.; Sun, H.; Shao, C. The Crucial Role of AR-V7 in Enzalutamide-Resistance of Castration-Resistant Prostate Cancer. Cancers (Basel) 2022, 14.
Zhao, J.; Ning, S.; Lou, W.; Yang, J. C.; Armstrong, C. M.; Lombard, A. P.; D'Abronzo, L. S.; Evans, C. P.; Gao, A. C.; Liu, C. Cross-Resistance Among Next-Generation Antiandrogen Drugs Through the AKR1C3/AR-V7 Axis in Advanced Prostate Cancer. Mol Cancer Ther 2020, 19, 1708-1718.
Liu, Y.; He, S.; Chen, Y.; Liu, Y.; Feng, F.; Liu, W.; Guo, Q.; Zhao, L.; Sun, H. Overview of AKR1C3: Inhibitor Achievements and Disease Insights. J Med Chem 2020, 63, 11305-11329.
Penning, T. M. Aldo-Keto Reductase (AKR) 1C3 inhibitors: a patent review. Expert Opin Ther Pat 2017, 27, 1329-1340.
Bauman, D. R.; Rudnick, S. I.; Szewczuk, L. M.; Jin, Y.; Gopishetty, S.; Penning, T. M. Development of nonsteroidal anti-inflammatory drug analogs and steroid carboxylates selective for human aldo-keto reductase isoforms: potential antineoplastic agents that work independently of cyclooxygenase isozymes. Mol Pharmacol 2005, 67, 60-68.
Adeniji, A.; Uddin, M. J.; Zang, T.; Tamae, D.; Wangtrakuldee, P.; Marnett, L. J.; Penning, T. M. Discovery of (R)-2-(6-Methoxynaphthalen-2-yl) butanoic Acid as a Potent and Selective Aldo-keto Reductase 1C3 Inhibitor. J Med Chem 2016, 59, 7431-7444.
Liedtke, A. J.; Adeniji, A. O.; Chen, M.; Byrns, M. C.; Jin, Y.; Christianson, D. W.; Marnett, L. J.; Penning, T. M. Development of potent and selective indomethacin analogues for the inhibition of AKR1C3 (Type 5 17beta-hydroxysteroid dehydrogenase/prostaglandin F synthase) in castrate-resistant prostate cancer. J Med Chem 2013, 56, 2429-2446.
Lolli, M. L.; Carnovale, I. M.; Pippione, A. C.; Wahlgren, W. Y.; Bonanni, D.; Marini, E.; Zonari, D.; Gallicchio, M.; Boscaro, V.; Goyal, P.; Friemann, R.; Rolando, B.; Bagnati, R.; Adinolfi, S.; Oliaro-Bosso, S.; Boschi, D. Bioisosteres of Indomethacin as Inhibitors of Aldo-Keto Reductase 1C3. ACS Med Chem Lett 2019, 10, 437-443.
Pippione, A. C.; Carnovale, I. M.; Bonanni, D.; Sini, M.; Goyal, P.; Marini, E.; Pors, K.; Adinolfi, S.; Zonari, D.; Festuccia, C.; Wahlgren, W. Y.; Friemann, R.; Bagnati, R.; Boschi, D.; Oliaro-Bosso, S.; Lolli, M. L. Potent and selective aldo-keto reductase 1C3 (AKR1C3) inhibitors based on the benzoisoxazole moiety: application of a bioisosteric scaffold hopping approach to flufenamic acid. Eur J Med Chem 2018, 150, 930-945.
Endo, S.; Oguri, H.; Segawa, J.; Kawai, M.; Hu, D.; Xia, S.; Okada, T.; Irie, K.; Fujii, S.; Gouda, H.; Iguchi, K.; Matsukawa, T.; Fujimoto, N.; Nakayama, T.; Toyooka, N.; Matsunaga, T.; Ikari, A. Development of Novel AKR1C3 Inhibitors as New Potential Treatment for Castration-Resistant Prostate Cancer. J Med Chem 2020, 63, 10396-10411.
Endo, S.; Matsunaga, T.; Kanamori, A.; Otsuji, Y.; Nagai, H.; Sundaram, K.; El-Kabbani, O.; Toyooka, N.; Ohta, S.; Hara, A. Selective inhibition of human type-5 17beta-hydroxysteroid dehydrogenase (AKR1C3) by baccharin, a component of Brazilian propolis. J Nat Prod 2012, 75, 716-721.
Endo, S.; Hu, D.; Matsunaga, T.; Otsuji, Y.; El-Kabbani, O.; Kandeel, M.; Ikari, A.; Hara, A.; Kitade, Y.; Toyooka, N. Synthesis of non-prenyl analogues of baccharin as selective and potent inhibitors for aldo-keto reductase 1C3. Bioorg Med Chem 2014, 22, 5220-5233.
Verma, K.; Gupta, N.; Zang, T.; Wangtrakluldee, P.; Srivastava, S. K.; Penning, T. M.; Trippier, P. C. AKR1C3 Inhibitor KV-37 Exhibits Antineoplastic Effects and Potentiates Enzalutamide in Combination Therapy in Prostate Adenocarcinoma Cells. Mol Cancer Ther 2018, 17, 1833-1845.
Kikuchi, A.; Furutani, T.; Azami, H.; Watanabe, K.; Niimi, T.; Kamiyama, Y.; Kuromitsu, S.; Baskin-Bey, E.; Heeringa, M.; Ouatas, T.; Enjo, K. In vitro and in vivo characterisation of ASP9521: a novel, selective, orally bioavailable inhibitor of 17beta-hydroxysteroid dehydrogenase type 5 (17betaHSD5; AKR1C3). Invest New Drugs 2014, 32, 860-870.
Loriot, Y.; Fizazi, K.; Jones, R. J.; Van den Brande, J.; Molife, R. L.; Omlin, A.; James, N. D.; Baskin-Bey, E.; Heeringa, M.; Baron, B.; Holtkamp, G. M.; Ouatas, T.; De Bono, J. S. Safety, tolerability and anti-tumour activity of the androgen biosynthesis inhibitor ASP9521 in patients with metastatic castration-resistant prostate cancer: multi-centre phase I/II study. Invest New Drugs 2014, 32, 995-1004.
Rizner, T. L.; Penning, T. M. Aldo-keto reductase 1C3-Assessment as a new target for the treatment of endometriosis. Pharmacol Res 2020, 152, 104446.
Zang, T.; Verma, K.; Chen, M.; Jin, Y.; Trippier, P. C.; Penning, T. M. Screening baccharin analogs as selective inhibitors against type 5 17beta-hydroxysteroid dehydrogenase (AKR1C3). Chem Biol Interact 2015, 234, 339-348.
Verma, K.; Zang, T.; Gupta, N.; Penning, T. M.; Trippier, P. C. Selective AKR1C3 Inhibitors Potentiate Chemotherapeutic Activity in Multiple Acute Myeloid Leukemia (AML) Cell Lines. ACS Med Chem Lett 2016, 7, 774-779.
Jonnalagadda, S. K.; Huwaimel, B. I.; Jonnalagadda, S.; Garrison, J. C.; Trippier, P. C. Access to Highly Strained Tricyclic Ketals Derived from Coumarins. J Org Chem 2022, 87, 4476-4482.
Verma, K.; Zang, T.; Penning, T. M.; Trippier, P. C. Potent and Highly Selective Aldo-Keto Reductase 1C3 (AKR1C3) Inhibitors Act as Chemotherapeutic Potentiators in Acute Myeloid Leukemia and T-Cell Acute Lymphoblastic Leukemia. J Med Chem 2019, 62, 3590-3616.
Kumari, S.; Carmona, A. V.; Tiwari, A. K.; Trippier, P. C. Amide Bond Bioisosteres: Strategies, Synthesis, and Successes. J Med Chem 2020, 63, 12290-12358.
Lassalas, P.; Gay, B.; Lasfargeas, C.; James, M. J.; Tran, V.; Vijayendran, K. G.; Brunden, K. R.; Kozlowski, M. C.; Thomas, C. J.; Smith, A. B., 3rd; Huryn, D. M.; Ballatore, C. Structure Property Relationships of Carboxylic Acid Isosteres. J Med Chem 2016, 59, 3183-3203.
Li, Y.; Chan, S. C.; Brand, L. J.; Hwang, T. H.; Silverstein, K. A.; Dehm, S. M. Androgen receptor splice variants mediate enzalutamide resistance in castration-resistant prostate cancer cell lines. Cancer Res 2013, 73, 483-489.
Smith, R.; Liu, M.; Liby, T.; Bayani, N.; Bucher, E.; Chiotti, K.; Derrick, D.; Chauchereau, A.; Heiser, L.; Alumkal, J.; Feiler, H.; Carroll, P.; Korkola, J. E. Enzalutamide response in a panel of prostate cancer cell lines reveals a role for glucocorticoid receptor in enzalutamide resistant disease. Sci Rep 2020, 10, 21750.
Penning, T. M.; Burczynski, M. E.; Jez, J. M.; Hung, C.-F.; Lin, H.-K.; Ma, H.; Moore, M.; Palackal, N.; Ratnam, K. Human 3alpha-hydroxysteroid dehydrogenase isoforms (AKR1C1-AKR1C4) of the aldo-keto reductase superfamily: functional plasticity and tissue distribution reveals roles in the inactivation and formation of male and female sex hormones. Biochem. J. 2000, 351, 67.
Adeniji, A. O.; Twenter, B. M.; Byrns, M. C.; Jin, Y.; Chen, M.; Winkler, J. D.; Penning, T. M. Development of potent and selective inhibitors of aldo-keto reductase 1C3 (type 5 17beta-hydroxysteroid dehydrogenase) based on N-phenyl-aminobenzoates and their structure-activity relationships. J Med Chem 2012, 55, 2311-2323.
Aldhafiri, W. N.; Chhonker, Y. S.; Zhang, Y.; Coutler, D. W.; McGuire, T. R.; Li, R.; Murry, D. J. Assessment of Tissue Distribution and Metabolism of MP1, a Novel Pyrrolomycin, in Mice Using a Validated LC-MS/MS Method. Molecules 2020, 25.
Bala, V.; Chhonker, Y. S.; Sleightholm, R. L.; Crawford, A. J.; Hollingsworth, M. A.; Murry, D. J. A rapid and sensitive bioanalytical LC-MS/MS method for the quantitation of a novel CDK5 inhibitor 20-223 (CP668863) in plasma: Application to in vitro metabolism and plasma protein-binding studies. Biomed Chromatogr 2020, 34, e4859.

1. A compound, or a pharmaceutically acceptable salt thereof, having a structure of formula (A): 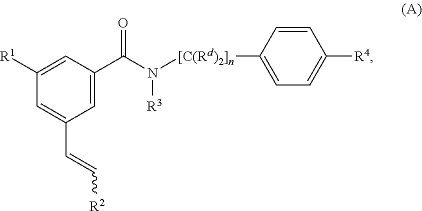
wherein
R¹ is selected from substituted or unsubstituted C₆-C₁₂aryl, substituted or unsubstituted C₂-C₆alkynyl, substituted or unsubstituted C₁-C₆alkyl, C₂-C₆alkenyl, substituted or unsubstituted C₃-C₆alkenyloxy, halo, substituted or unsubstituted C₆-C₁₀aryl-C₁-C₃alkyleneoxy, C₆-C₁₀aryloxy, and substituted or unsubstituted C₅-C₁₀heteroaryl having 1-4 ring heteroatoms selected from N, O, and S;
wherein R¹ is optionally substituted with one or more substituents selected from C₆-C₁₀aryl; 5-15 membered heteroaryl having 1-4 ring heteroatoms selected from N, O, and S; formyl, C₃-C₆cycloalkyl, halo, cycloheteroalkyl-alkylene, hydroxy-C₁-C₃alkylene, methoxymethyl, phenyoxy, cyano, C₁-C₆alkyl, C₁-C₆haloalkyl, and 5-7 membered fused cycloheteroalkyl having 1 to 3 ring heteroatoms selected from N, O, and S;
R² is selected from CO₂Rₐ, CONR^{b}R^{c}, wherein each R^{a}, R^{b}, and R^{c} is independently H or C₁-C₆alkyl;
each R³ and R⁴ is independently H or C₁-C₆alkyl;
each R^{d} is independently H or C₁-C₃alkyl; and
n is 1, 2, or 3.

2. The compound or pharmaceutically acceptable salt of claim 1, wherein R¹ is selected from:

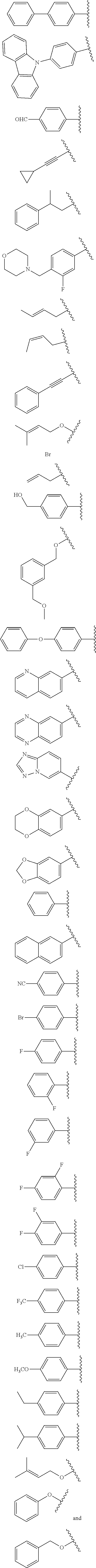

3. The compound or pharmaceutically acceptable salt of claim 1, wherein R¹ is selected from:

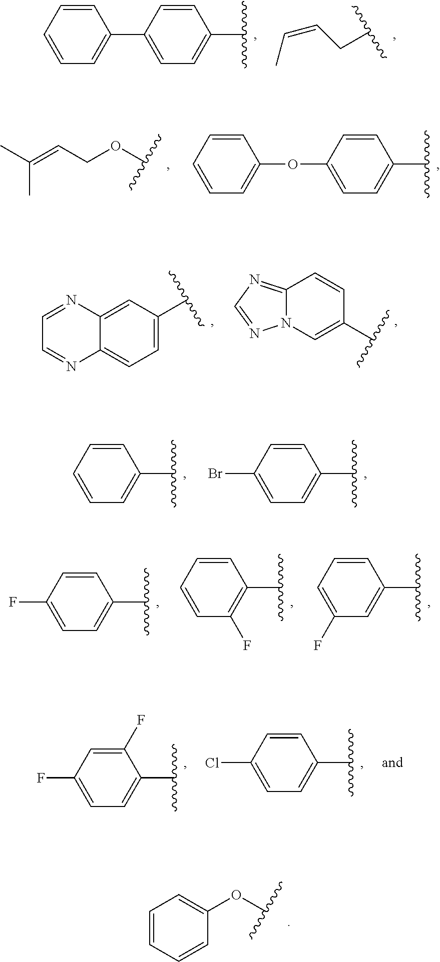

4. The compound or pharmaceutically acceptable salt of claim 1, wherein R¹ is selected from:

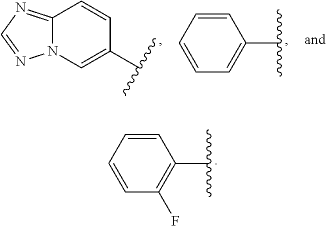 [SMILES: *c1ccc2ncnn2c1 ; *c1ccccc1 ; *c1ccccc1F]

5. The compound or pharmaceutically acceptable salt of claim 1, wherein R² is selected from —CO₂H, —CO₂CH₃, —CO₂CH₂CH₃, —CO₂(CH₂)₂CH₃, —CO₂C(CH₃)₃, —CONH₂, —CON(CH₃)₂, and —CN.

6. The compound or pharmaceutically acceptable salt of claim 1, wherein R² is CO₂H.

7. The compound or pharmaceutically acceptable salt of claim 1, wherein R³ is H.

8. The compound or pharmaceutically acceptable salt of claim 1, wherein R⁴ is C₁-C₆alkyl.

9. The compound or pharmaceutically acceptable salt of claim 8, wherein R⁴ is methyl.

10. The compound or pharmaceutically acceptable salt of claim 1, wherein one or more R^{d} is ethyl.

11. The compound or pharmaceutically acceptable salt of claim 1, wherein formula (A) is selected from:

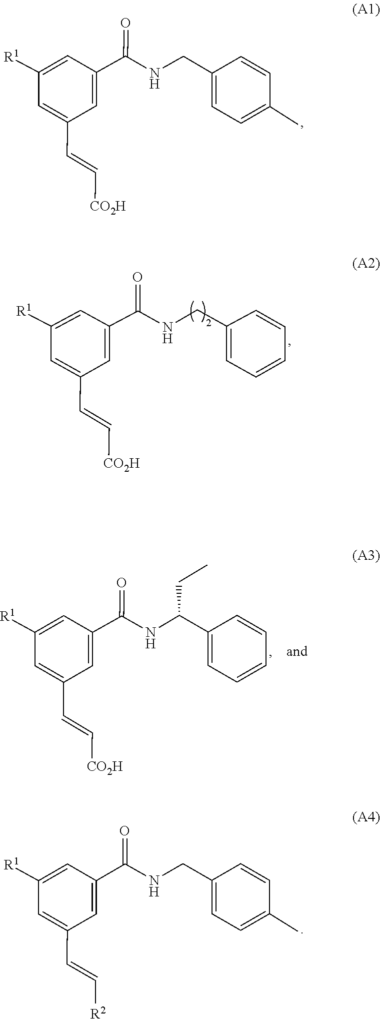

12. The compound or pharmaceutically acceptable salt of claim 11, wherein R² of formula A-4 is selected from —CN, —CONH₂, —CON(CH₃)₂, and —CO₂CH₃.

13. The compound or pharmaceutically acceptable salt of claim 1, wherein the structure of formula (A) is selected from the group consisting of

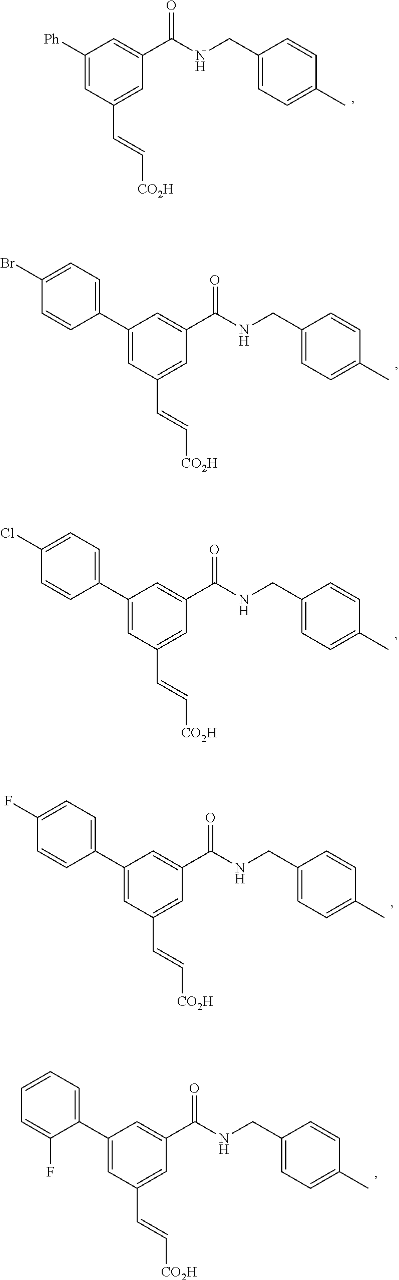 [SMILES: Cc1ccc(CNC(=O)c2cc(/C=C/C(=O)O)cc(-c3ccccc3)c2)cc1 ; Cc1ccc(CNC(=O)c2cc(/C=C/C(=O)O)cc(-c3ccc(Br)cc3)c2)cc1 ; Cc1ccc(CNC(=O)c2cc(/C=C/C(=O)O)cc(-c3ccc(Cl)cc3)c2)cc1 ; Cc1ccc(CNC(=O)c2cc(/C=C/C(=O)O)cc(-c3ccc(F)cc3)c2)cc1 ; Cc1ccc(CNC(=O)c2cc(/C=C/C(=O)O)cc(-c3ccccc3F)c2)cc1]

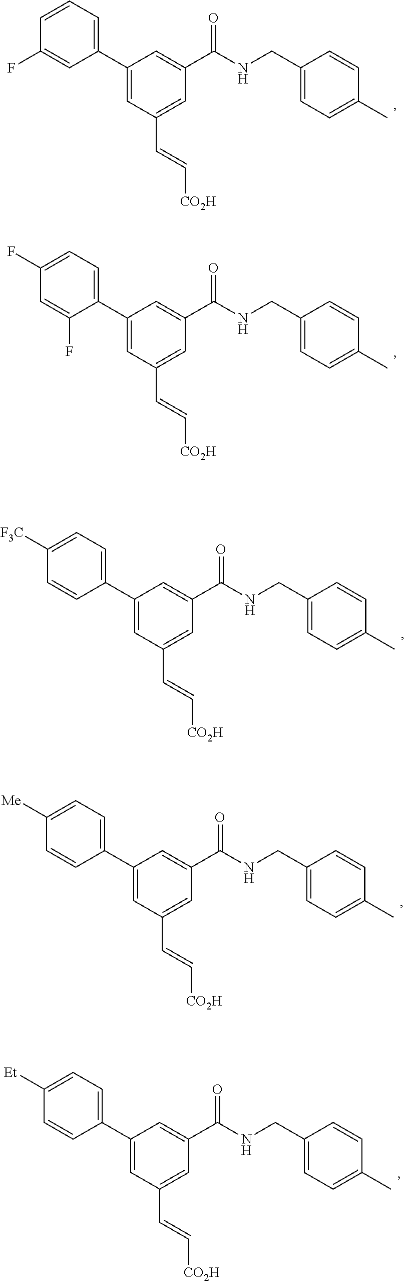 [SMILES: Cc1ccc(CNC(=O)c2cc(/C=C/C(=O)O)cc(-c3cccc(F)c3)c2)cc1 ; Cc1ccc(CNC(=O)c2cc(/C=C/C(=O)O)cc(-c3ccc(F)cc3F)c2)cc1 ; Cc1ccc(CNC(=O)c2cc(/C=C/C(=O)O)cc(-c3ccc(C(F)(F)F)cc3)c2)cc1 ; Cc1ccc(CNC(=O)c2cc(/C=C/C(=O)O)cc(-c3ccc(C)cc3)c2)cc1 ; CCc1ccc(-c2cc(/C=C/C(=O)O)cc(C(=O)NCc3ccc(C)cc3)c2)cc1]

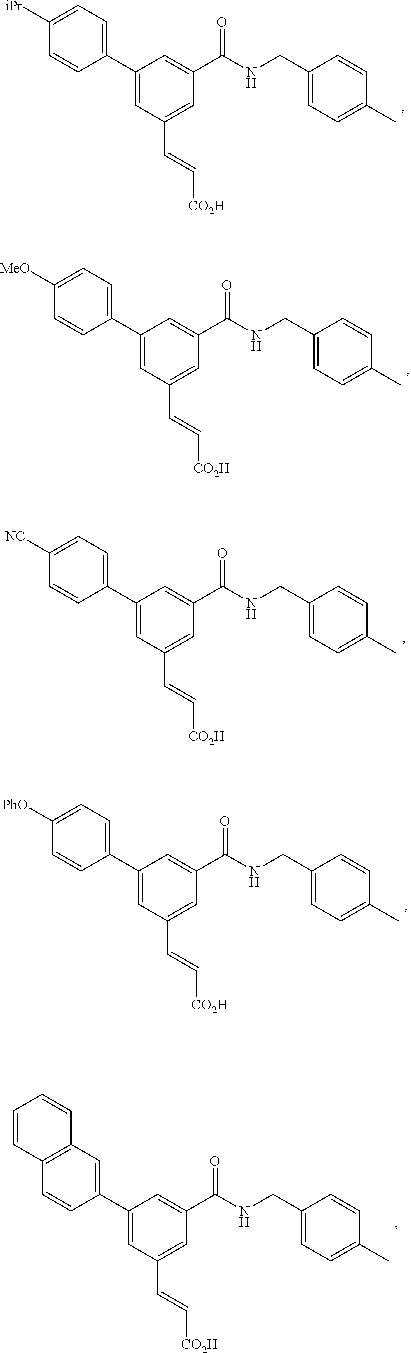 [SMILES: Cc1ccc(CNC(=O)c2cc(/C=C/C(=O)O)cc(-c3ccc(C(C)C)cc3)c2)cc1 ; COc1ccc(-c2cc(/C=C/C(=O)O)cc(C(=O)NCc3ccc(C)cc3)c2)cc1 ; Cc1ccc(CNC(=O)c2cc(/C=C/C(=O)O)cc(-c3ccc(C#N)cc3)c2)cc1 ; Cc1ccc(CNC(=O)c2cc(/C=C/C(=O)O)cc(-c3ccc(Oc4ccccc4)cc3)c2)cc1 ; Cc1ccc(CNC(=O)c2cc(/C=C/C(=O)O)cc(-c3ccc4ccccc4c3)c2)cc1]

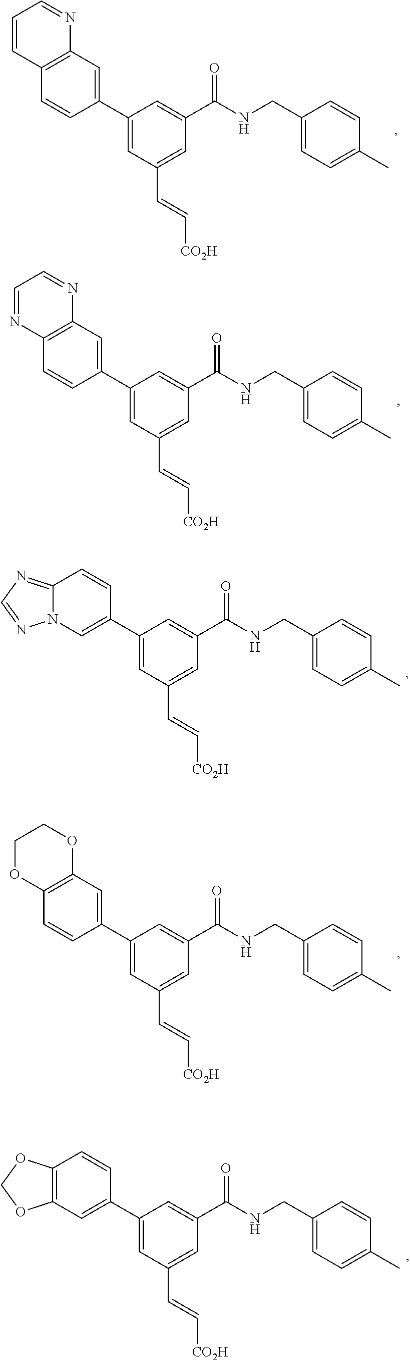 [SMILES: Cc1ccc(CNC(=O)c2cc(/C=C/C(=O)O)cc(-c3ccc4cccnc4c3)c2)cc1 ; Cc1ccc(CNC(=O)c2cc(/C=C/C(=O)O)cc(-c3ccc4nccnc4c3)c2)cc1 ; Cc1ccc(CNC(=O)c2cc(/C=C/C(=O)O)cc(-c3ccc4ncnn4c3)c2)cc1 ; Cc1ccc(CNC(=O)c2cc(/C=C/C(=O)O)cc(-c3ccc4c(c3)OCCO4)c2)cc1 ; Cc1ccc(CNC(=O)c2cc(/C=C/C(=O)O)cc(-c3ccc4c(c3)OCO4)c2)cc1]

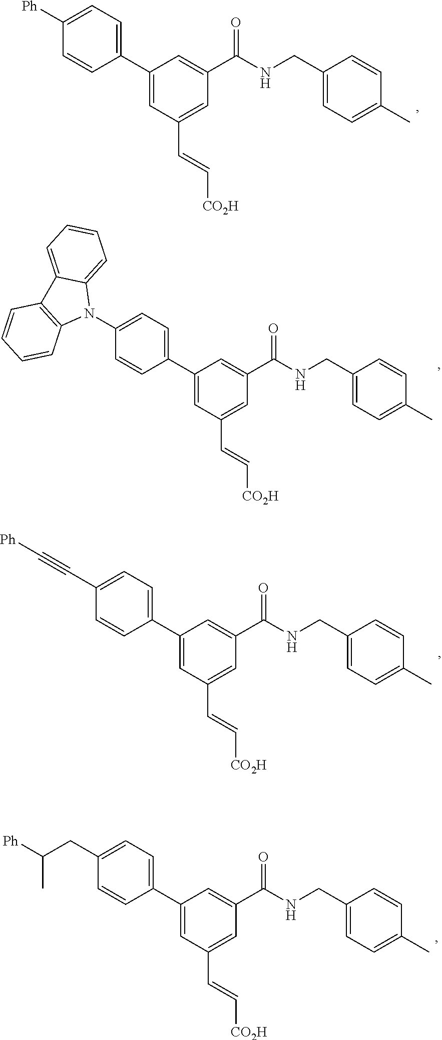 [SMILES: Cc1ccc(CNC(=O)c2cc(/C=C/C(=O)O)cc(-c3ccc(-c4ccccc4)cc3)c2)cc1 ; Cc1ccc(CNC(=O)c2cc(/C=C/C(=O)O)cc(-c3ccc(-n4c5ccccc5c5ccccc54)cc3)c2)cc1 ; Cc1ccc(CNC(=O)c2cc(/C=C/C(=O)O)cc(-c3ccc(C#Cc4ccccc4)cc3)c2)cc1 ; Cc1ccc(CNC(=O)c2cc(/C=C/C(=O)O)cc(-c3ccc(CC(C)c4ccccc4)cc3)c2)cc1]

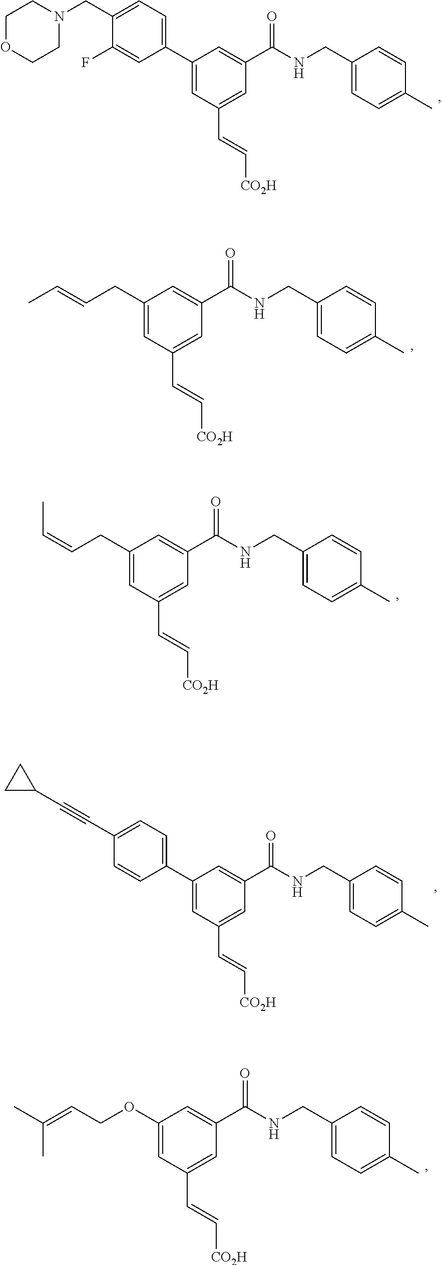

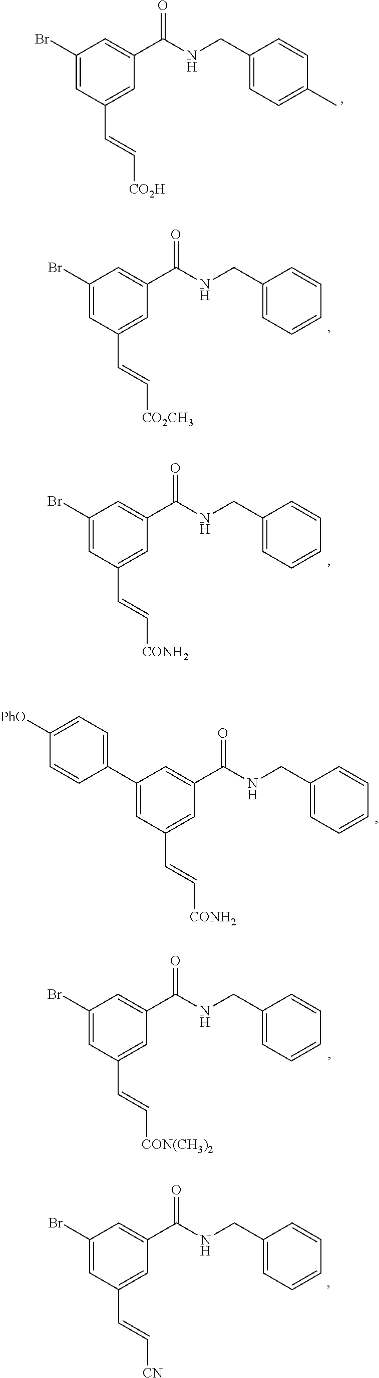

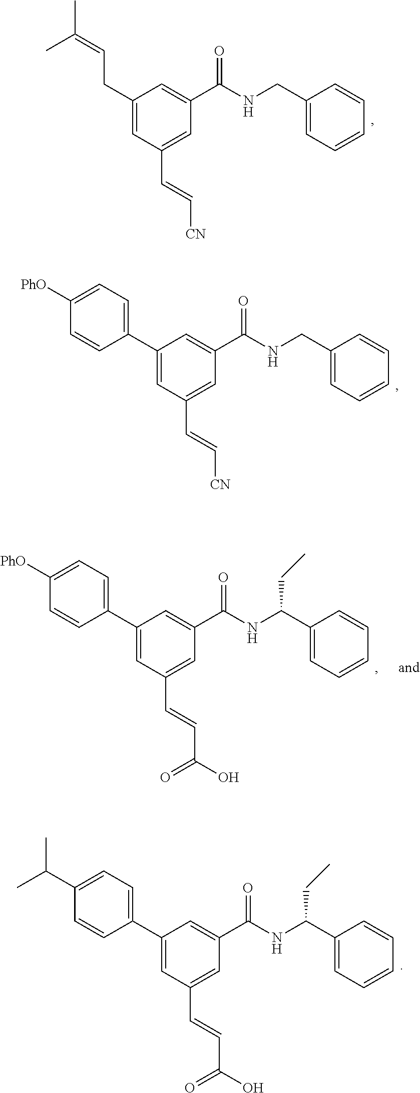 [SMILES: CC(C)=CCc1cc(/C=C/C#N)cc(C(=O)NCc2ccccc2)c1 ; N#C/C=C/c1cc(C(=O)NCc2ccccc2)cc(-c2ccc(Oc3ccccc3)cc2)c1 ; CC[C@@H](NC(=O)c1cc(/C=C/C(=O)O)cc(-c2ccc(Oc3ccccc3)cc2)c1)c1ccccc1 ; CC[C@@H](NC(=O)c1cc(/C=C/C(=O)O)cc(-c2ccc(C(C)C)cc2)c1)c1ccccc1]

14. A pharmaceutical composition comprising the compound of formula (A) or pharmaceutically acceptable salt thereof according to claim 1 and a pharmaceutically acceptable carrier or excipient.

15. The pharmaceutical composition of claim 14, wherein the compound of formula (A) is selected from a compound of

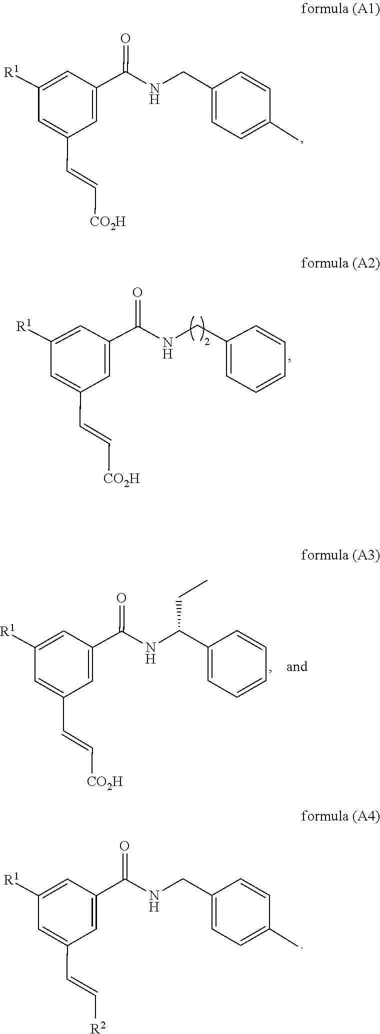

16. The pharmaceutical composition of claim 14 comprising a compound or pharmaceutically acceptable salt thereof selected from the group consisting of

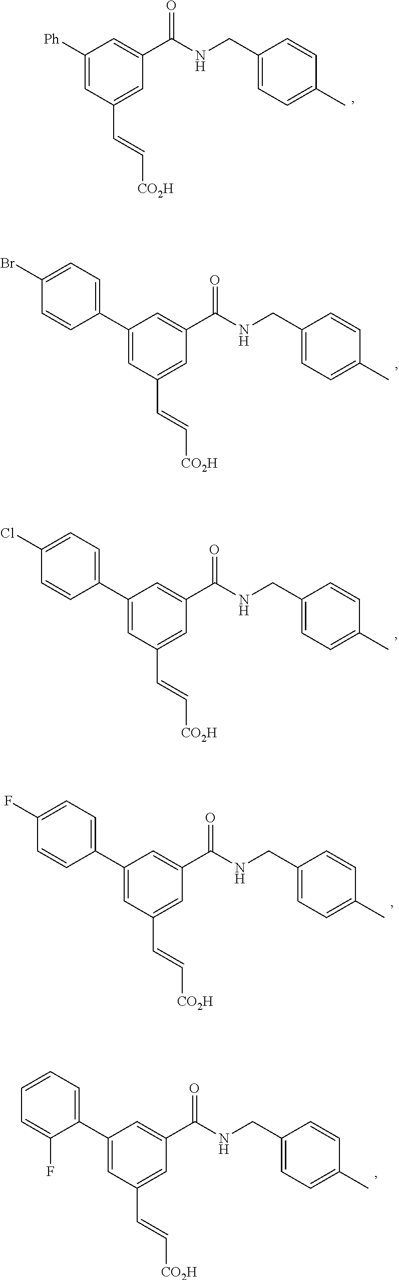 [SMILES: Cc1ccc(CNC(=O)c2cc(/C=C/C(=O)O)cc(-c3ccccc3)c2)cc1 ; Cc1ccc(CNC(=O)c2cc(/C=C/C(=O)O)cc(-c3ccc(Br)cc3)c2)cc1 ; Cc1ccc(CNC(=O)c2cc(/C=C/C(=O)O)cc(-c3ccc(Cl)cc3)c2)cc1 ; Cc1ccc(CNC(=O)c2cc(/C=C/C(=O)O)cc(-c3ccc(F)cc3)c2)cc1 ; Cc1ccc(CNC(=O)c2cc(/C=C/C(=O)O)cc(-c3ccccc3F)c2)cc1]

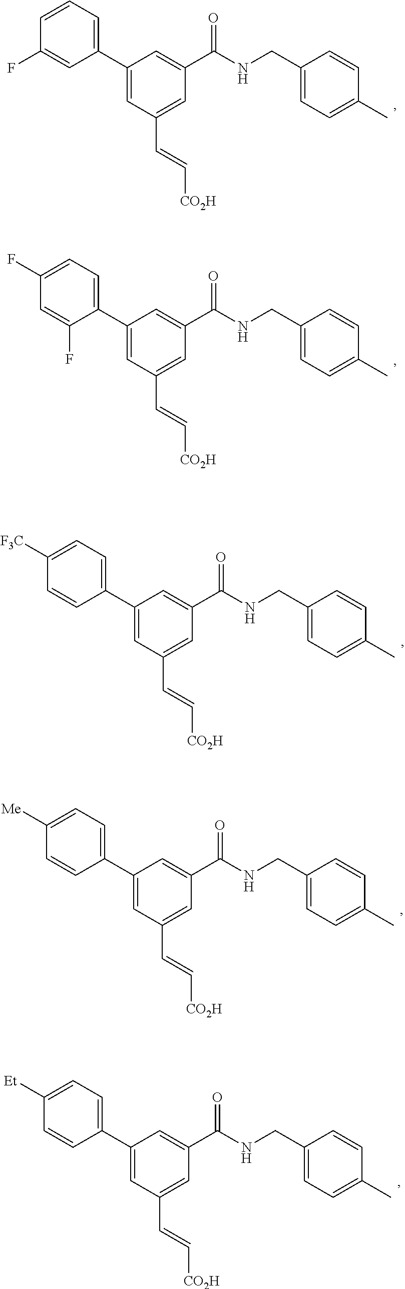 [SMILES: Cc1ccc(CNC(=O)c2cc(/C=C/C(=O)O)cc(-c3cccc(F)c3)c2)cc1 ; Cc1ccc(CNC(=O)c2cc(/C=C/C(=O)O)cc(-c3ccc(F)cc3F)c2)cc1 ; Cc1ccc(CNC(=O)c2cc(/C=C/C(=O)O)cc(-c3ccc(C(F)(F)F)cc3)c2)cc1 ; Cc1ccc(CNC(=O)c2cc(/C=C/C(=O)O)cc(-c3ccc(C)cc3)c2)cc1 ; CCc1ccc(-c2cc(/C=C/C(=O)O)cc(C(=O)NCc3ccc(C)cc3)c2)cc1]

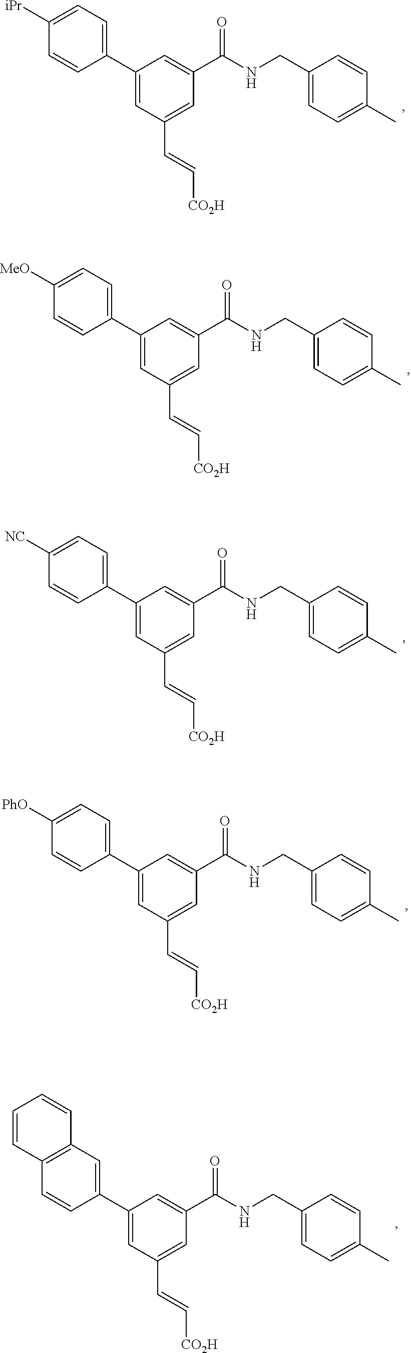 [SMILES: Cc1ccc(CNC(=O)c2cc(/C=C/C(=O)O)cc(-c3ccc(C(C)C)cc3)c2)cc1 ; COc1ccc(-c2cc(/C=C/C(=O)O)cc(C(=O)NCc3ccc(C)cc3)c2)cc1 ; Cc1ccc(CNC(=O)c2cc(/C=C/C(=O)O)cc(-c3ccc(C#N)cc3)c2)cc1 ; Cc1ccc(CNC(=O)c2cc(/C=C/C(=O)O)cc(-c3ccc(Oc4ccccc4)cc3)c2)cc1 ; Cc1ccc(CNC(=O)c2cc(/C=C/C(=O)O)cc(-c3ccc4ccccc4c3)c2)cc1]

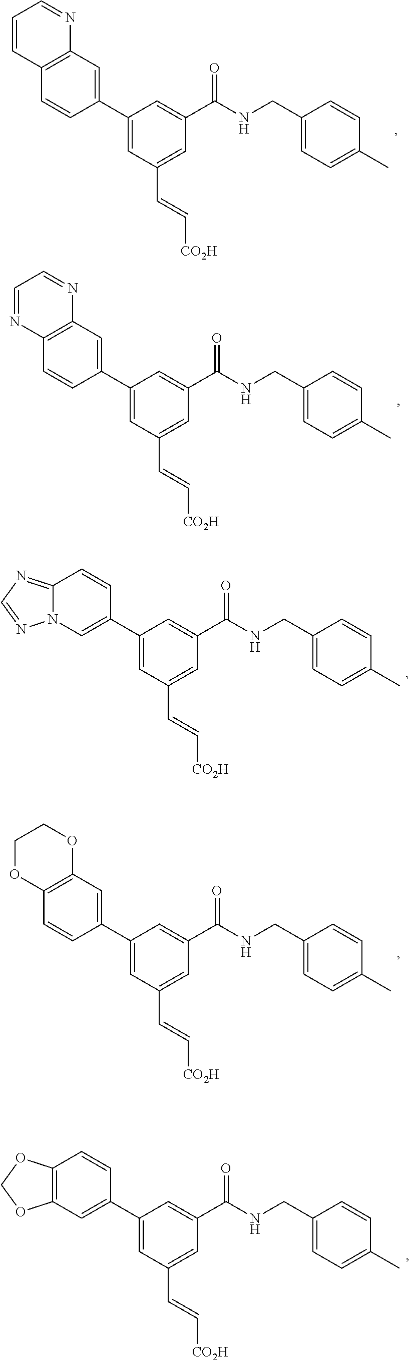 [SMILES: Cc1ccc(CNC(=O)c2cc(/C=C/C(=O)O)cc(-c3ccc4cccnc4c3)c2)cc1 ; Cc1ccc(CNC(=O)c2cc(/C=C/C(=O)O)cc(-c3ccc4nccnc4c3)c2)cc1 ; Cc1ccc(CNC(=O)c2cc(/C=C/C(=O)O)cc(-c3ccc4ncnn4c3)c2)cc1 ; Cc1ccc(CNC(=O)c2cc(/C=C/C(=O)O)cc(-c3ccc4c(c3)OCCO4)c2)cc1 ; Cc1ccc(CNC(=O)c2cc(/C=C/C(=O)O)cc(-c3ccc4c(c3)OCO4)c2)cc1]

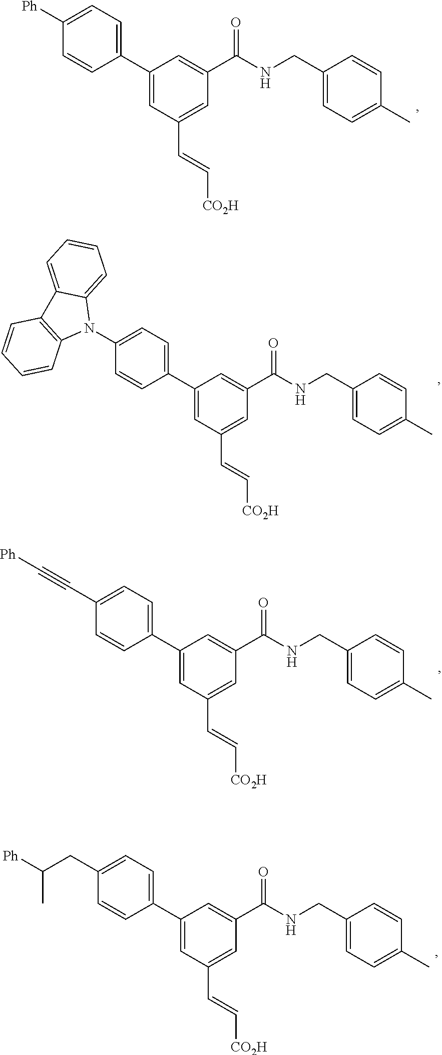 [SMILES: Cc1ccc(CNC(=O)c2cc(/C=C/C(=O)O)cc(-c3ccc(-c4ccccc4)cc3)c2)cc1 ; Cc1ccc(CNC(=O)c2cc(/C=C/C(=O)O)cc(-c3ccc(-n4c5ccccc5c5ccccc54)cc3)c2)cc1 ; Cc1ccc(CNC(=O)c2cc(/C=C/C(=O)O)cc(-c3ccc(C#Cc4ccccc4)cc3)c2)cc1 ; Cc1ccc(CNC(=O)c2cc(/C=C/C(=O)O)cc(-c3ccc(CC(C)c4ccccc4)cc3)c2)cc1]

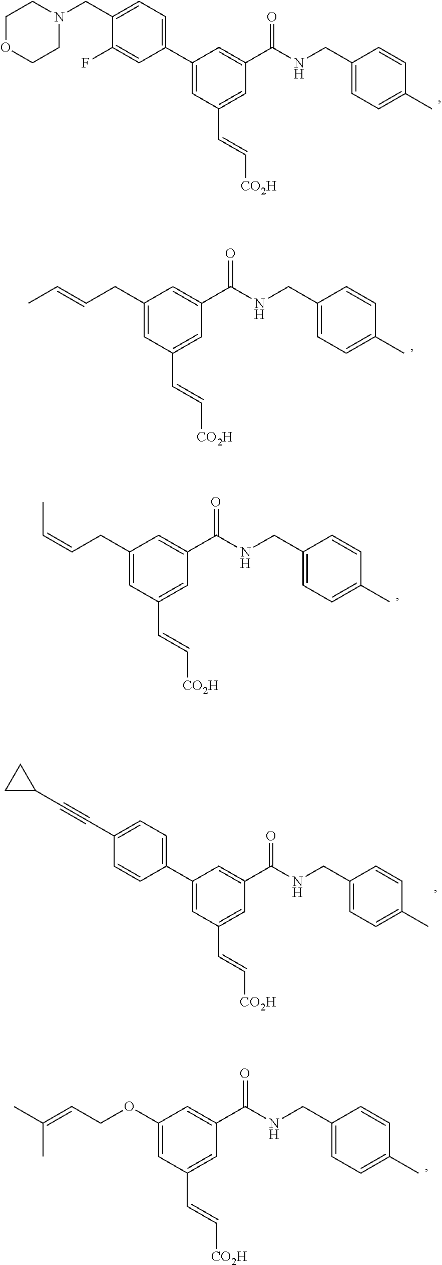

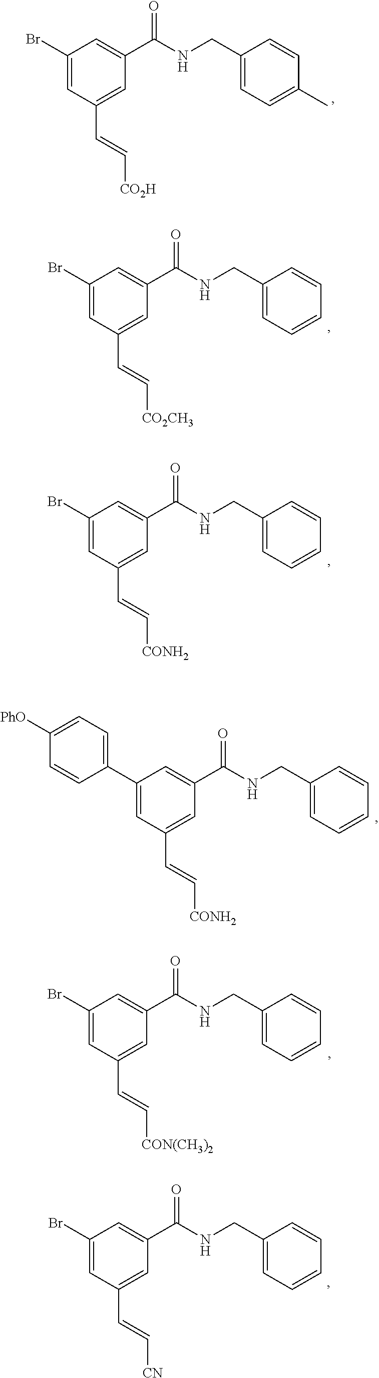

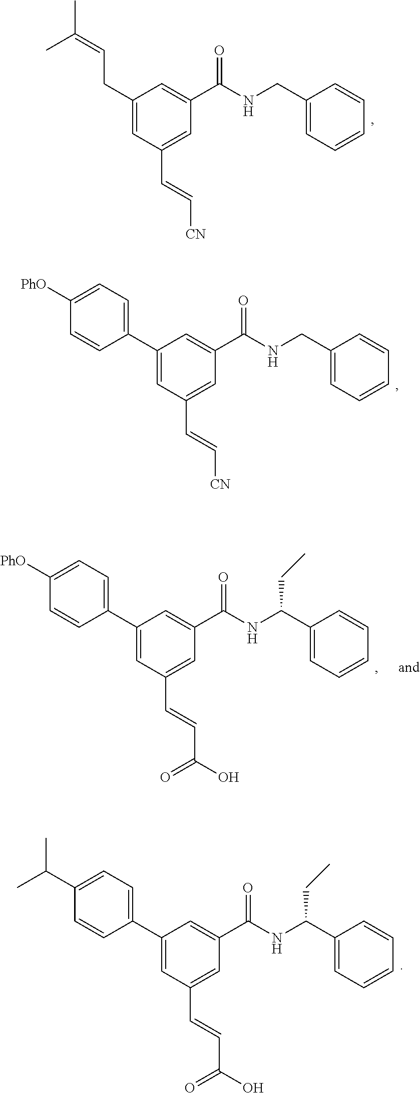 [SMILES: CC(C)=CCc1cc(/C=C/C#N)cc(C(=O)NCc2ccccc2)c1 ; N#C/C=C/c1cc(C(=O)NCc2ccccc2)cc(-c2ccc(Oc3ccccc3)cc2)c1 ; CC[C@@H](NC(=O)c1cc(/C=C/C(=O)O)cc(-c2ccc(Oc3ccccc3)cc2)c1)c1ccccc1 ; CC[C@@H](NC(=O)c1cc(/C=C/C(=O)O)cc(-c2ccc(C(C)C)cc2)c1)c1ccccc1]

17. A method of inhibiting AKR1C3 comprising contacting AKR1C3 with a compound or pharmaceutically acceptable salt of formula (A) according to claim 1 in an amount effective to inhibit AKR1C3.

18. A method of treating and/or preventing a disease in a subject in need thereof, comprising administering to the subject a therapeutically effective amount of a compound of formula (A) or pharmaceutically acceptable salt according to claim 1.

19. (canceled)

20. The method of claim 18, wherein the disease is a cancer is selected from leukemia (e.g., acute lymphoblastic leukemia, acute myeloid leukemia, chronic lymphocytic leukemia, chronic myelogenous leukemia, T-cell acute lymphoblastic leukemia), lymphoma (e.g., Hodgkin lymphoma, non-Hodgkin lymphoma), multiple myeloma, breast cancer, prostate cancer, pancreatic cancer, colon cancer, thyroid cancer, bladder cancer, liver cancer, neuroblastoma, brain cancers (e.g., gliomas, meningiomas, pituitary adenomas, etc.), lung cancer, ovarian cancer, stomach cancer, skin cancer (melanoma), cervical cancer, testicular cancer, kidney cancer, carcinoid tumors, bone cancer, and endometrial cancer.

21.-30. (canceled)

31. A method of enhancing or potentiating the effectiveness of an active agent comprising administering the active agent in combination with an effective amount of a compound or pharmaceutically acceptable salt according to claim 1.

32.-42. (canceled)
